(19) United States
(12) Patent Application Publication
(10) Pub. No.: US 2026/0049098 A1
(43) Pub. Date: Feb. 19, 2026

(54) SYNTHETIC PROCESSES AND INTERMEDIATES FOR PREPARING THERAPEUTIC AZAKETOLIDES

(71) Applicant: ZIKANI THERAPEUTICS, INC., Watertown, MA (US)

(72) Inventors: Shuhao SHI, Madison, CT (US); He ZHAO, Madison, CT (US); Jianmin MAO, Winchester, MA (US); Xiben LI, Lexington, MA (US); Wenying WANG, Lexington, MA (US); Yoshitaka ICHIKAWA, Cambridge, MA (US); Roger B. CLARK, Lexington, MA (US)

(21) Appl. No.: 19/102,932

(22) PCT Filed: Aug. 10, 2023
(86) PCT No.: PCT/US2023/071989

§ 371 Date: Feb. 11, 2025

Related U.S. Application Data
(60) Provisional application No. 63/397,206, filed on Aug. 11, 2022.

(51) Int. Cl. C07H 17/08; C07H 1/00; C07H 15/04; C07H 15/26

(52) U.S. Cl. CPC C07H 17/08; C07H 1/00; C07H 15/04; C07H 15/26

(57) ABSTRACT

The present invention relates to a process for producing compounds of formula I and/or formula I′. The present invention also relates to a process of selectively cleaving the main ring of a compound of formula IV to provide the starting materials for the synthesis of a compound of formula I or formula I′. The present invention further relates to the intermediates of the processes described herein.

### TECHNICAL FIELD OF THE INVENTION

The present invention relates to a process for producing compounds of formula I and/or formula I′. The present invention also relates to a process of selectively cleaving the main ring of a compound of formula IV to provide the starting materials for the synthesis of a compound of formula I or formula I′. The present invention further relates to the intermediates of the processes described herein.

### BACKGROUND OF THE INVENTION

Nonsense mutations are mutations where a stop codon (UAA, UAG or UGA) replaces an amino acid-coding codon, leading to premature termination of translation and eventually to truncated inactive proteins. The Human Gene Mutation Database reports the occurrence of thousands of disease-causing mutations, approximately 12% of which are single point (nonsense) mutations that result in a premature termination codon. (Krawczak M, et al., Hum Mutat. 2000, 15, 45-51.; Mort, et al., M. Hum. Mutat. 2008, 29, 1037-47). Nonsense mutations that result in truncated proteins have been demonstrated to account for many forms of genetic disease including cancer, hemophilia, Tay-Sachs, lysosomal storage disorders or mucopolysaccharidoses such as Hurler Syndrome, Duchenne muscular dystrophy, ataxia telangiectasia, Rett syndrome, various inherited retinopathies, and cystic fibrosis.

Effective treatments for genetic diseases caused by nonsense mutations remain elusive. As a result, the discovery and development of new compounds effective against nonsense and/or frameshift mutations giving rise to premature termination codons and thus useful for the treatment of genetic diseases and disorders caused by nonsense mutations remains an ongoing unmet need. The azaketolides are one such class of recently discovered compounds that can be used to treat such conditions. A need remains for processes for making such compounds.

### SUMMARY OF THE INVENTION

These and other needs are met by the present invention which is directed to processes for making azaketolides that can be used to treat genetic diseases caused by nonsense mutations.

In one aspect, the invention includes a process for preparing a compound of formula I or formula I′:

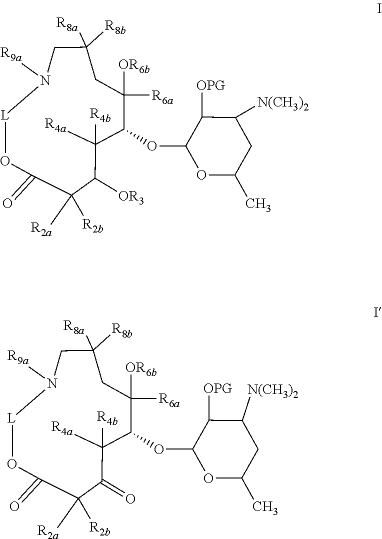

or a pharmaceutically acceptable salt thereof, comprising:
(a1) intramolecular cyclization of a compound of formula A, wherein O-LG is a leaving group and PG is a protecting group, to form a compound of formula I:

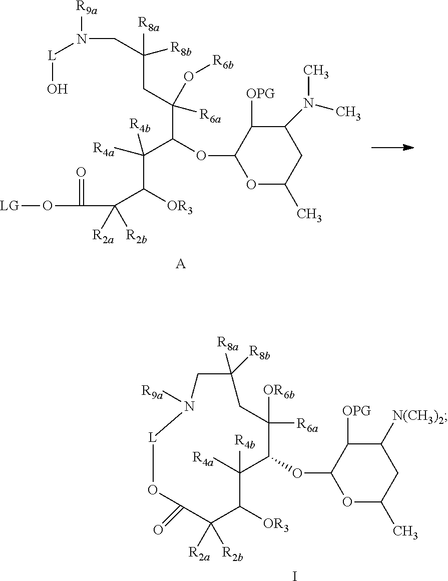

(b1) intramolecular cyclization of a compound of formula B, wherein PG is a protecting group, to form a compound of formula I′:

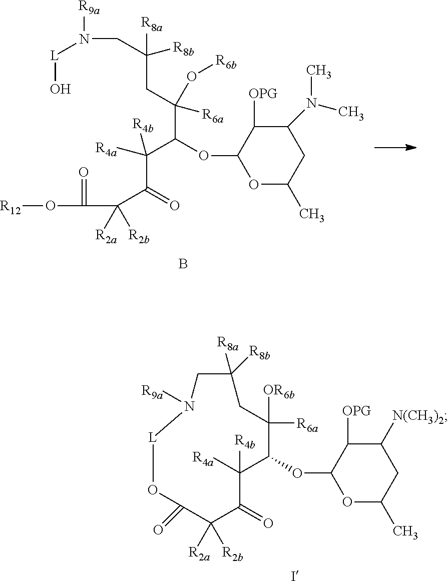

wherein:
one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is selected from the group consisting of halo, optionally substituted C₁₋₁₀ alkyl, optionally substituted C₁₋₁₀ alkoxy, and optionally substituted C₂₋₄₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
R₃ is H, a suitable protecting group selected from acyl, carbamoyl, alkyl ether, or silyl ether protecting groups, or

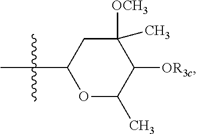

wherein Rac is H or a protecting group;
each of R₄ₐ and R_{4b} is independently selected from the group consisting of —H, and optionally substituted C₁₋₁₀alkyl;
R₆ₐ is optionally substituted C₁₋₁₀ alkyl;
R_{6b} is —H, optionally substituted C₁₋₁₀ alkyl, optionally substituted C₁₋₁₀ hydroxyalkyl, and optionally substituted allyl;
R₈ₐ and R_{8b} are each independently selected from the group consisting of —H and optionally substituted C₁₋₁₀alkyl;
R₉ₐ is selected from the group consisting of —H and optionally substituted C₁₋₁₀ alkyl;
R₁₂ is a C₁₋₆ alkyl; and
L is a C₂₋₅ optionally substituted alkylene.

In one aspect, the invention includes a process for preparing a compound of formula B-9 from a compound of formula IV, comprising contacting the compound of formula IV with ozone, optionally in the presence of an acid, to form a compound of formula B-9:

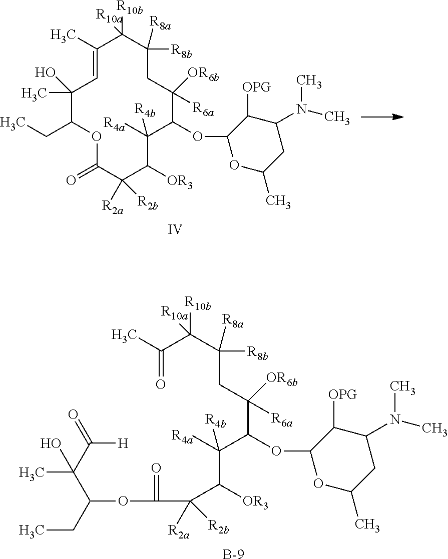

wherein:
one of R₁₀ₐ and R_{10b} is OH and the other is H or R₁₀ₐ and R_{10b} together form C═O.

Also provided are compounds useful as intermediates in the process which are depicted in the schemes and experimental examples provided herein.

What is also provided is a compound depicted in Table 1.

The process disclosed herein starts from readily available macrolides found in nature. As such, the invention is directed to the chemical synthesis of azalides using natural products as starting points and solves the problem of preparing new compounds with therapeutic activity in a minimum number of controlled steps.

### DETAILED DESCRIPTION

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety, including U.S. Pat. Publ. No. 2013/0090326. In case of conflict, the present specification, including these definitions, will control.

The terms “a,” “an,” and “the” as used herein not only include aspects with one member, but also include aspects with more than one member.

The term “about” as used herein means “approximately” and is used to modify a numerical value indicates a defined range around that value. If “X” were the value, “about X” would generally indicate a value from 0.95× to 1.05×. Any reference to “about X” specifically indicates at least the values X, 0.95×, 0.96×, 0.97×, 0.98×, 0.99×, 1.01×, 1.02×, 1.03×, 1.04×, and 1.05×. Thus, “about X” is intended to teach and provide written description support for a claim limitation of, e.g., “0.98×.” When the quantity “X” only includes whole-integer values (e.g., “X carbons”), “about X” indicates from (X−1) to (X+1). In this case, “about X” as used herein specifically indicates at least the values X, X−1, and X+1.

“About” and “approximately” may be used interchangeably. When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

The following abbreviations and terms have the indicated meanings throughout:

| Abbreviation | Meaning |
|---|---|
| AcOH | acetic acid |
| br | broad |
| ° C. | degrees Celsius |
| conc | concentrated |
| d | doublet |
| dd | doublet of doublet |
| dt | doublet of triplet |
| DCM | dichloromethane |
| DIEA or DIPEA | N,N-di-isopropyl-N-ethylamine |
| DMA | N,N-dimethylacetamide |
| DME | 1,2-dimethoxyethane |
| DMF | N,N-dimethylformamide |
| DMSO | dimethyl sulfoxide |
| dppf | 1,1′-bis(diphenylphosphano)ferrocene |
| EI | Electron Impact ionization |
| equiv | equivalents |
| g | gram(s) |
| GC/MS | gas chromatography/mass spectrometry |
| h or hr | hour(s) |
| HATU | 2-(1H-7-azabenzotriazol-1-yl)-1,1,3,3-tetramethyl |
| | uronium hexafluorophosphate |
| HPLC | high pressure liquid chromatography |
| L | liter(s) |
| LC/MS | liquid chromatography/mass spectrometry |
| M | molar or molarity |
| m | Multiplet |
| MeOH | methanol |
| mg | milligram(s) |
| MHz | megahertz (frequency) |
| min | minute(s) |
| mL | milliliter(s) |
| μL | microliter(s) |
| μM | micromolar |
| μmol | micromole(s) |
| mM | Millimolar |
| mmol | millimole(s) |
| mol | mole(s) |
| MS | mass spectral analysis |
| Ms | mesyl |
| N | normal or normality |
| nM | Nanomolar |
| NMR | nuclear magnetic resonance spectroscopy |
| q | Quartet |
| quant | quantitative |
| rt | Room temperature |
| s | Singlet |
| t or tr | Triplet |
| THF | tetrahydrofuran |
| Ts | tosyl |

The symbol “-” means a single bond, “=” means a double bond, “=” means a triple bond, “ [symbol] ” means a single or double bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent Formula, the “ [symbol] ” symbol will be used at the end of the bond which was theoretically cleaved in order to separate the group from its parent structural Formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

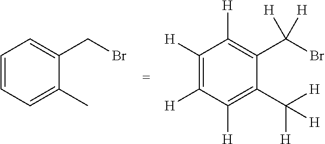

If a group “R” is depicted as “floating” on a ring system, as for example in the following Formula.

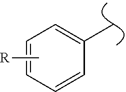

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused or bridged ring system, as for example in the following Formulas.

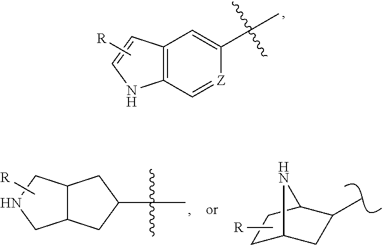

then, unless otherwise defined, a substituent “R” may reside on any atom of the fused or bridged ring system, assuming replacement of a depicted hydrogen (for example the —NH— in the Formula above), implied hydrogen (for example as in the Formula above, where the hydrogens are not shown but understood to be present), or expressly defined hydrogen (for example where in the Formula above, “Z” equals ═CH—) from one of the ring atoms, so long as a stable structure is formed. In the example depicted, the “R” group may reside on either the 5-membered or the 6-membered ring of the fused or bridged ring system.

When a group “R” is depicted as existing on a ring system containing saturated carbons, as for example in the following Formula

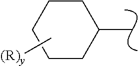

where, in this example, “y” can be more than one, assuming each replaces a currently depicted, implied, or expressly defined hydrogen on the ring; then, unless otherwise defined, where the resulting structure is stable, two “R's” may reside on the same carbon. In another example, two R's on the same carbon, including that carbon, may form a ring, thus creating a spirocyclic ring structure with the depicted ring as for example in the following Formula

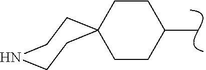

The term “acyl” as used herein includes an alkanoyl, aroyl, heterocycloyl, or heteroaroyl group as defined herein. Examples of acyl groups include, but are not limited to, acetyl, benzoyl, and nicotinoyl.

The term “alkanoyl” as used herein includes an alkyl-C(O)— group wherein the alkyl group is as defined herein. Examples of alkanoyl groups include, but are not limited to, acetyl and propanoyl.

The term “agent” as used herein includes a compound or mixture of compounds that, when added to a composition, tend to produce a particular effect on the composition's properties. For example, a composition comprising a thickening agent is likely to be more viscous than an otherwise identical comparative composition that lacks the thickening agent.

The term “alkenyl” as used herein includes a straight or branched chain hydrocarbon containing at least one carbon-carbon double bond. The chain may contain an indicated number of carbon atoms. For example, “C₁-C₁₂ alkenyl” indicates that the group may have from 1 to 12 (inclusive) carbon atoms and at least one carbon-carbon double bond. When the indicated number of carbon atoms is 1, then the Cᵢ alkenyl is double bonded to a carbon (i.e., a carbon equivalent to an oxo group). In certain aspects, the chain includes 1 to 12, about 2 to 15, about 2 to 12, about 2 to 8, or about 2 to 6 carbon atoms. An alkenyl group can be preferably one stereoisomer (i.e., cis- or, alternatively, trans-). Examples of an alkenyl group may include, but are not limited to, ethenyl (i.e., vinyl), allyl, propenyl, butenyl, crotyl, pentenyl, hexenyl, heptenyl, octenyl, nonenyl, decenyl, dodecenyl, cyclopentenyl, cyclohexenyl, 2-isopentenyl, allenyl, butadienyl, pentadienyl, 3-(1,4-pentadienyl), and hexadienyl.

An alkenyl group can be unsubstituted or optionally substituted. When optionally substituted, one or more hydrogen atoms of the alkenyl group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no hydrogen atom substituent on the carbon-carbon double bond is replaced by a hydroxy, amino, or thio group. In some aspects, the alkenyl group is unsubstituted or not optionally substituted.

“Alkenylene” as used herein includes an alkenyl group that is substituted at two points. An example is but-2-enylene (—CH₂CH═CHCH₂—) and the like.

The term “alkyl” as used herein includes an aliphatic hydrocarbon chain that may be straight chain or branched. The chain may contain an indicated number of carbon atoms: For example, C₁-C₁₀ indicates that the group may have from 1 to 10 (inclusive) carbon atoms in it. If not otherwise indicated, an alkyl group contains from 1 to about 20 carbon atoms. In some aspects, alkyl groups have 1 to about 10 carbon atoms. In some aspects, alkyl groups (“lower alkyl”) have 1 to 8, 1 to 6, or 1 to 3 carbon atoms in the chain. Examples may include, but are not limited to, methyl, ethyl, propyl, isopropyl (iPr), 1-butyl, 2-butyl, isobutyl (iBu), tert-butyl, pentyl, 2-methylbutyl, 1,1-dimethylpropyl, hexyl, heptyl, octyl, nonyl, decyl, docecyl, cyclopentyl, or cyclohexyl.

An alkyl group can be unsubstituted or optionally substituted. When optionally substituted, one or more hydrogen atoms of the alkyl group (e.g., from 1 to 4, from 1 to 2, or 1) may be replaced with a moiety independently selected from the group consisting of fluoro, hydroxy, alkoxy, amino, alkylamino, acylamino, thio, and alkylthio. In some aspects, the alkyl group is unsubstituted or not optionally substituted.

“Alkylene” as used herein includes an alkyl group that is substituted at two points. An example is methylene (—CH₂—), propylene (—CH₂CH₂CH₂—), and the like.

The term “alkoxy” as used herein includes a straight or branched chain saturated or unsaturated hydrocarbon containing at least one oxygen atom in an ether group (e.g., EtO—). The chain may contain an indicated number of carbon atoms. For example, “C₁-C₁₂ alkoxy” indicates that the group may have from 1 to 12 (inclusive) carbon atoms and at least one oxygen atom. Examples of a C₁-C₁₂ alkoxy group include, but are not limited to, methoxy, ethoxy, isopropoxy, butoxy, n-pentoxy, isopentoxy, neopentoxy, and hexoxy.

An alkoxy group can be unsubstituted or optionally substituted. When optionally substituted, one or more hydrogen atoms of the alkoxy group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no hydrogen atom alpha to the ether oxygen is replaced by a hydroxy, amino, or thio group. In some aspects, the alkoxy group is unsubstituted or not optionally substituted.

The term “alkynyl” as used herein includes a straight, branched, or cyclic hydrocarbon containing at least one carbon-carbon triple bond. Examples may include, but are not limited to, ethynyl, propargyl, propynyl, butynyl, pentynyl, hexynyl, heptynyl, octynyl, nonynyl, decynyl, or decynyl.

“Alkynylene” as used herein includes an alkynyl group that is substituted at two points. An example is 2-butynylene (—CH₂CCCH₂—) and the like.

An alkynyl group can be unsubstituted or optionally substituted. When optionally substituted, one or more hydrogen atoms of the alkynyl group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no sp-hybridized hydrogen atom substituent is replaced by a hydroxy, amino, or thio group. In some aspects, the alkynyl group is unsubstituted or not optionally substituted.

The term “aryl” as used herein includes cyclic aromatic carbon ring systems containing from 6 to 18 carbons. Examples of an aryl group include, but are not limited to, phenyl, naphthyl, anthracenyl, tetracenyl, biphenyl and phenanthrenyl.

An aryl group can be unsubstituted or optionally substituted. When optionally substituted, one or more hydrogen atoms of the aryl group (e.g., from 1 to 5, from 1 to 2, or 1) may be replaced with a moiety independently selected from the group consisting of alkyl, cyano, acyl, halo, hydroxy, alkoxy, amino, alkylamino, acylamino, thio, and alkylthio. In some aspects, the alkoxy group is unsubstituted or not optionally substituted.

The term “arylalkyl” or “aralkyl” as used herein includes an alkyl group as defined herein where at least one hydrogen substituent has been replaced with an aryl group as defined herein. Examples include, but are not limited to, benzyl, 1-phenylethyl, 4-methylbenzyl, and 1,1,-dimethyl-1-phenylmethyl.

An arylalkyl or aralkyl group can be unsubstituted or optionally substituted as per its component groups. For example, but without limitation, the aryl group of an arylalkyl group can be substituted, such as in 4-methylbenzyl. In some aspects, the group is unsubstituted or not optionally substituted, especially if including a defined substituent, such as a hydroxyalkyl or alkylaminoalkoxy group.

The term “cycloalkyl” as used herein includes non-aromatic saturated monocyclic or multicyclic ring system that may contain an indicated number of carbon atoms. For example, C₃-C₁₂ indicates that the group may have from 3 to 12 (inclusive) carbon atoms in it. If not otherwise indicated, a cycloalkyl group includes about 3 to about 20 carbon atoms. In some aspects, cyclo alkyl groups have 3 to about 12 carbon atoms in the group. In some aspects, cycloalkyl groups have 3 to about 7 carbon atoms in the group. Examples may include, but are not limited to, cyclopropyl, cyclobutyl, cyclopentyl, cyclohexyl, 4,4-dimethylcyclohexyl, and cycloheptyl.

A cycloalkyl group can be unsubstituted or optionally substituted. When optionally substituted, one or more hydrogen atoms of the cycloalkyl group (e.g., from 1 to 4, from 1 to 2, or 1) may be replaced with a moiety independently selected from the group consisting of fluoro, hydroxy, alkoxy, amino, alkylamino, acylamino, thio, and alkylthio. In some aspects, a substituted cycloalkyl group can incorporate an exo- or endocyclic alkene (e.g., cyclohex-2-en-1-yl). In some aspects, a cycloalkyl group is unsubstituted or not optionally substituted.

As used herein, “fluoroalkyl” includes an alkyl group wherein the alkyl group includes one or more fluoro-substituents. Examples include, but are not limited to, trifluoromethyl.

As used herein, “geminal” substitution includes two or more substituents that are directly attached to the same atom. An example is 3,3-dimethyl substitution on a cyclohexyl or spirocyclohexyl ring.

As used herein, “halo” or “halogen” includes fluoro, chloro, bromo, and iodo.

The term “heteroaryl” or “heterocycloaryl” includes mono and bicyclic aromatic groups of about 4 to about 14 ring atoms (e.g., 4 to 10 or 5 to 10 atoms) containing at least one heteroatom. Heteroatom as used in the term heteroaryl refers to oxygen, sulfur and nitrogen. A nitrogen atom of a heteroaryl is optionally oxidized to the corresponding N-oxide. Examples include, but are not limited to, pyrazinyl, furanyl, thienyl, pyridyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridine, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, and benzothiazolyl.

The term “heteroarylene” or “heterocycloarylene” as used herein includes a heteroaryl group that is substituted at two points.

An heteroaryl group can be unsubstituted or optionally substituted. When optionally substituted, one or more hydrogen atoms of the heteroaryl group (e.g., from 1 to 5, from 1 to 2, or 1) may be replaced with a moiety independently selected from the group consisting of alkyl, cyano, acyl, halo, hydroxy, alkoxy, amino, alkylamino, acylamino, thio, and alkylthio. In some aspects, the heteroaryl group is unsubstituted or not optionally substituted.

The term “heteroaroyl” as used herein includes a heteroaryl-C(O)— group wherein heteroaryl is as defined herein. Heteroaroyl groups include, but are not limited to, thiophenoyl, nicotinoyl, pyrrol-2-ylcarbonyl, and pyridinoyl.

The term “heterocycloalkyl” may be used interchangeably herein, and as used herein includes a heterocyclyl-C(O)— group wherein heterocyclyl is as defined herein. Examples include, but are not limited to, N-methyl prolinoyl and tetrahydrofuranoyl.

As used herein, “heterocyclyl” (heterocyclo; heterocyclic; heterocycloalkyl) includes a non-aromatic saturated ring of about 3 to about 8 ring atoms (e.g., 5 to about 10 ring atoms, or 3 to about 6 ring atoms), in which one or more of the atoms in the ring system is an element or elements other than carbon, e.g., nitrogen, oxygen or sulfur. A heterocyclyl group optionally comprises at least one sp²-hybridized atom (e.g., a ring incorporating an carbonyl, endocyclic olefin, or exocyclic olefin). In some embodiments, a nitrogen or sulfur atom of the heterocyclyl is optionally oxidized to the corresponding N-oxide, S-oxide or S,S-dioxide. The monocyclic heterocycle means a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyridazin-3(2H)-onyl, pyridin-2(1H)-onyl, pyrrolinyl, pyrrolidinyl, tetrahydrofuranyl, tetrahydropyranyl, tetrahydropyridinyl, tetrahydropyrimidinyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The term “heterocycloalkylene” as used herein includes a heterocyclyl (heterocyclo; heterocyclic) group that is substituted at two points.

The term “heterocyclyl” also includes multicyclic rings such as a bicyclic heterocycle, or a tricyclic heterocycle which may be in a fused, bridged, or spiro orientation. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, 3-azabicyclo[3.1.0]hexane, 3-azabicyclo[4.1.0]heptane, 3-azabicyclo[3.2.0]heptane, (3aR,6aS)-hexahydro-1H-2λ2-cyclopenta[c]pyrrole, (3aR,7aS)-octahydro-2λ2-isoindole.

Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms.

A heterocycyl group can be unsubstituted or optionally substituted. When optionally substituted, one or more hydrogen atoms of the group (e.g., from 1 to 4, from 1 to 2, or 1) may be replaced with a moiety independently selected from the group consisting of fluoro, hydroxy, alkoxy, amino, alkylamino, acylamino, thio, and alkylthio. In some aspects, a substituted heterocycyl group can incorporate an exo- or endocyclic alkene (e.g., cyclohex-2-en-1-yl). In some aspects, the heterocycyl group is unsubstituted or not optionally substituted.

The monocyclic, bicyclic, and tricyclic heterocycles are connected to the parent molecular moiety through any carbon atom or any nitrogen atom contained within the rings, and can be unsubstituted or substituted.

As used herein, the term “hydrophilic moiety” or “hydrophilic group” includes a moiety or a functional group that has a strong affinity to water. Examples may include, but are not limited to, a charged moiety, such as a cationic moiety or an anionic moiety, or a polar uncharged moiety, such as an alkoxy group or an amine group.

As used herein, the term “hydroxyalkyl” includes an alkyl group where at least one hydrogen substituent has been replaced with an alcohol (—OH) group. In certain aspects, the hydroxyalkyl group has one alcohol group. In certain aspects, the hydroxyalkyl group has one or two alcohol groups, each on a different carbon atom. In certain aspects, the hydroxyalkyl group has 1, 2, 3, 4, 5, or 6 alcohol groups. Examples may include, but are not limited to, hydroxymethyl, 2-hydroxyethyl, and 1-hydroxyethyl.

When any two substituent groups or any two instances of the same substituent group are “independently selected” from a list of alternatives, the groups may be the same or different. For example, if R^{a} and R^{b} are independently selected from the group consisting of alkyl, fluoro, amino, and hydroxyalkyl, then a molecule with two R^{a} groups and two R^{b} groups could have all groups be an alkyl group (e.g., four different alkyl groups). Alternatively, the first R^{a} could be alkyl, the second R^{a} could be fluoro, the first R^{b} could be hydroxyalkyl, and the second R^{b} could be amino (or any other substituents taken from the group). Alternatively, both R^{a} and the first R^{b} could be fluoro, while the second R^{b} could be alkyl (i.e., some pairs of substituent groups may be the same, while other pairs may be different).

“Protecting Group” means “amino protecting group,” which is a protecting group that is suitable for preventing undesired reactions at an amino nitrogen. Representative amino-protecting groups include, but are not limited to, formyl; acyl groups, for example alkanoyl groups, such as acetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBDMS); and the like. Representative oxygen protecting groups include, but are not limited to silyl groups, such as TMS and TMDMS; esters, such as formate, acetate, and benzoate (Bz); ethers such as methoxymethyl (MOM), tetrahydropyranyl (THP), and benzyl (Bn)

“Yield” for each of the reactions described herein is expressed as a percentage of the theoretical yield.

“Subject and “patient” are used interchangeably. A “subject” or “patient” for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17*^{h} ed., Mack Publishing Company, Easton, PA, 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine. “Platin(s),” and “platin-containing agent(s)” include, for example, cisplatin, carboplatin, and oxaliplatin.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

The phrase “genetic disease”, as used herein, means a genetic disorder, genetic disease, genetic condition or genetic syndrome.

“Preventing” or “prevention” of a disease, disorder, or syndrome includes inhibiting the disease from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome.

“Treating” or “treatment” of a disease, disorder, or syndrome, as used herein, includes (i) inhibiting the disease, disorder, or syndrome, i.e., arresting its development; and (ii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

As used herein, the term “oxidizing agent” refers to a substance or reagent that tends to bring about oxidation by being reduced and gaining electrons. Examples of oxidizing agents are selected from, but not limited to, Oxygen (O₂), Ozone (O₃), Hydrogen peroxide (H₂O₂) and other inorganic peroxides, Fenton's reagent, Fluorine (F₂), chlorine (Cl₂), and other halogens, Nitric acid (HNO₃) and nitrate compounds, Sulfuric acid (H₂SO₄), Peroxydisulfuric acid (H₂S₂O₈), Peroxymonosulfuric acid (H₂SO₅), Chlorite, chlorate, perchlorate, and other analogous halogen compounds, Hypochlorite and other hypohalite compounds, including household bleach (NaClO), Hexavalent chromium compounds such as chromic and dichromic acids and chromium trioxide, pyridinium chlorochromate (PCC), and chromate/dichromate compounds, Permanganate compounds such as potassium permanganate, Sodium perborate, Nitrous oxide (N₂O), Nitrogen dioxide/Dinitrogen tetroxide (NO₂/N₂O₄), Potassium nitrate (KNO₃), the oxidizer in black powder, Sodium bismuthate, and sodium periodate (NaIO₄).

CFTR modulator types as used herein are drugs or compounds that target the underlying defect in the systic bibrosis transmembrance conductance regulator (CFTR) protein. Three types of modulators are potentiators, correctors, and amplifiers. [accessed on May 23, 2019, Cystic fibrosis Foundation https://www.cff.org/Research/Developing-New-Treatments/CFTR-Modulator-Types/].

Potentiator, such as the drug ivacaftor (Kalydeco®), works on residual function and splice mutations where an insufficient amount of normal CFTR protein is present by holding the gate open so chloride can flow through.

Correctors help the CFTR protein with two copies of the F508del mutation to form the right 3-D shape so that it is able to move—or traffic—to the cell surface.

Amplifiers increase the amount of CFTR protein that the cell makes. Many CFTR mutations produce insufficient CFTR protein. If the cell made more CFTR protein, potentiators and correctors would be able to allow even more chloride to flow across the cell membrane.

### Embodiments

In one aspect, the invention includes a process for preparing a compound of formula I or formula I′:

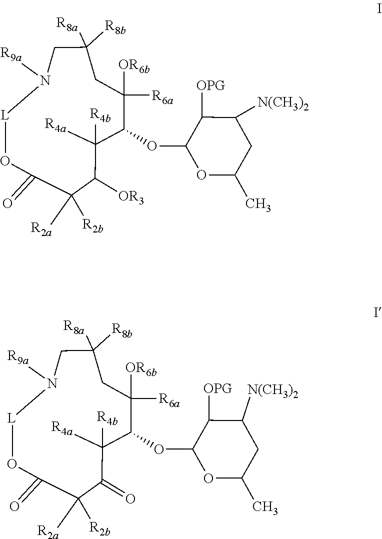

or a pharmaceutically acceptable salt thereof, comprising:
(a1) intramolecular cyclization of a compound of formula A, wherein O-LG is a leaving group and PG is a protecting group, to form a compound of formula I:

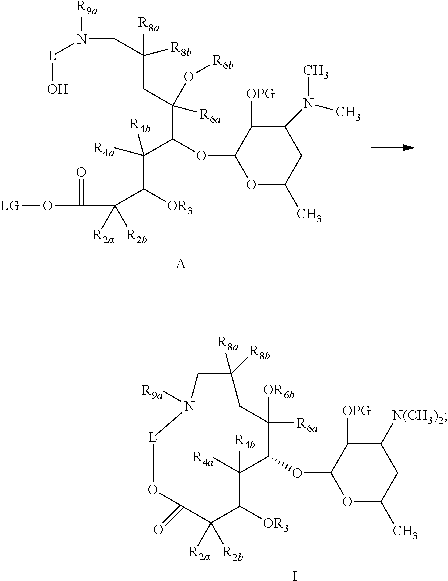

(b1) intramolecular cyclization of a compound of formula B, wherein PG is a protecting group, to form a compound of formula I′:

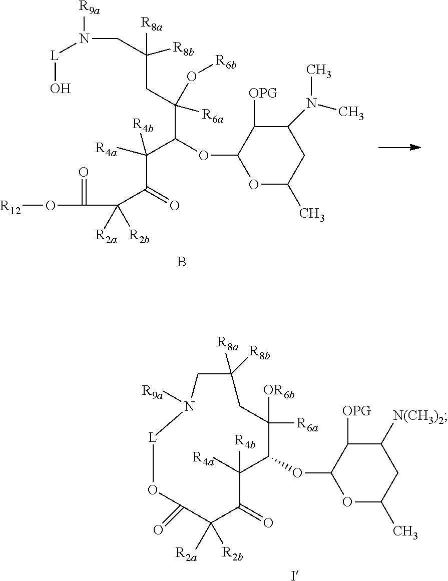

wherein:
one of R₂ₐ, and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is selected from the group consisting of halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
R₃ is H, a suitable protecting group selected from acyl, carbamoyl, alkyl ether, or silyl ether protecting groups, or

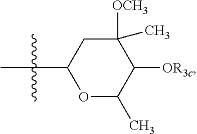

wherein R_{3c} is H or a protecting group;
each of R₄ₐ and R_{4b} is independently selected from the group consisting of —H, and optionally substituted C₁₋₁₀alkyl;
R₆ₐ is optionally substituted C₁₋₁₀ alkyl;
R_{6b} is —H, optionally substituted C₁₋₁₀ alkyl, optionally substituted C₁₋₁₀ hydroxyalkyl, and optionally substituted allyl;
R₈ₐ and R_{8b} are each independently selected from the group consisting of —H and optionally substituted C₁₋₁₀alkyl;
R₉ₐ is selected from the group consisting of —H and optionally substituted C₁₋₁₀ alkyl;
R₁₂ is C₁₋₆ alkyl; and
L is a C₂₋₅ optionally substituted alkylene.

In some embodiments, R₃ is H or

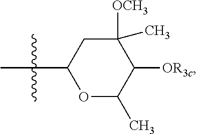

wherein R_{3c} is H or a protecting group. In some further embodiments, R₃ is H. In some other further embodiments, R₃ is

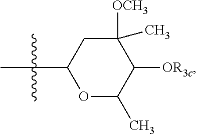

wherein R_{3c} is H or a protecting group.

In one embodiment, the intramolecular cyclization of a compound of formula B proceeds in the presence of a solvent at neutral conditions.

In another embodiment, the intramolecular cyclization of a compound of formula B proceeds at a temperature above 120° C.

In one embodiment, the intramolecular cyclization of a compound of formula B proceeds at a temperature from about 50° C. to about 175° C.

In another embodiment, the intramolecular cyclization of a compound of formula B proceeds at a temperature from about 60° C. to about 150° C. in the presence of m-xylene.

In another embodiment, the intramolecular cyclization of a compound of formula B proceeds at a temperature from about 100° C. to about 150° C. in the presence of m-xylene.

In a further embodiment, the intramolecular cyclization of a compound of formula B proceeds at a temperature from about 130° C. to about 140° C. in the presence of m-xylene.

In one embodiment, the intramolecular cyclization occurs in an aprotic solvent. Examples of aprotic solvents include, but are not limited to DMF, DMSO, acetone, DCM, ethyl acetate, toluene, or diethyl ether. In a further embodiment, the intramolecular cyclization occurs in the presence of 4-dimethylaminopyridine (DMAP). In another further embodiment, the intramolecular cyclization occurs in the presence of a base. In yet a further embodiment, the base is an organic base such as trimethylamine, diisopropylethylamine, DBU, and others known to those having skill in the art.

In one embodiment, the process of the invention comprises the step of converting a compound of formula A-1 to the compound of formula A:

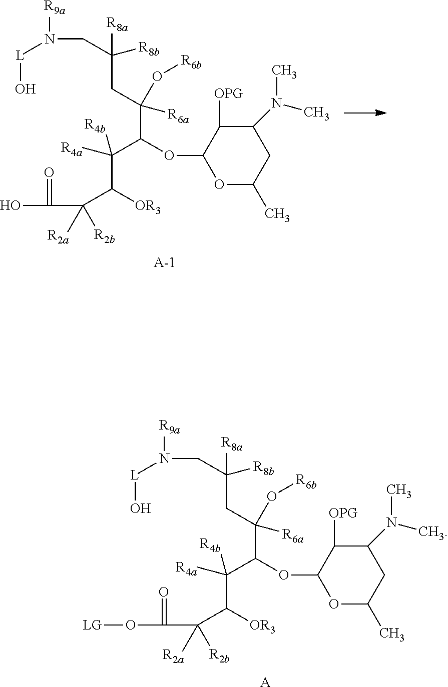

In one embodiment, LG is R′—(C═O)—.

In one embodiment, R′ is optionally substituted phenyl.

In one embodiment, the process comprises a base.

In a further embodiment, the base is an amine base selected from the group consisting of 1,5,7-Triazabicyclo(4.4.0)dec-5-ene (TBD), 7-Methyl-1,5,7-triazabicyclo(4.4.0)dec-5-ene (MTBD), 1,8-Diazabicyclo[5.4.0]undec-7-ene (DBU), 1,5-Diazabicyclo[4.3.0]non-5-ene (DBN), 1,1,3,3-Tetramethylguanidine (TMG), Quinuclidine, 2,2,6,6-Tetramethylpiperidine (TMP), Pempidine (PMP), Tributlyamine, Triethylamine, diisopropylethylamine (DIEA), 1,4-Diazabicyclo[2.2.2]octan (TED), Collidine, 2,6-Lutidine (2,6-Dimethylpyridine).

In another embodiment, the process further comprises the step of converting a compound of formula A-2, wherein Rₓ is a C₁₋₆ alkyl, optionally substituted with C₁₋₄ alkyl, hydroxyl, oxo, COOH, COO(C₁₋₆ alkyl), amino, alkylamino, halo, or cyano, to a compound of formula A-1:

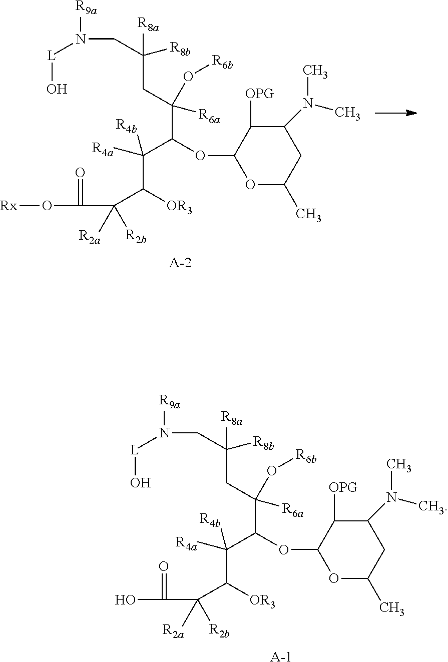

In one embodiment, Rₓ is methyl, substituted methyl, ethyl, substituted ethyl, benzyl, substituted benzyl, or t-butyl. In a further embodiment, Rₓ is methyl, benzyl, pentyl substituted with one or more of oxo, hydroxyl, and methyl. In still a further embodiment, Rₓ is benzyl. In some other embodiments, Rₓ is pent-3-yl-2-one, 2-hydroxy-2-methyl-pentanal-3-yl, and 1,2-dihydroxy-2-methyl-pentan-3-yl.

In another embodiment, the process further comprises the step of converting a compound of formula A-3 to a compound of formula A-2:

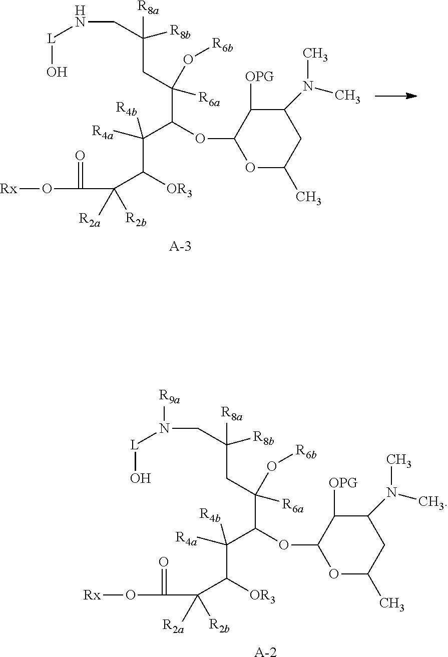

In another embodiment, the process further comprises the step of converting the compound of formula A-4 to a compound of formula A-3

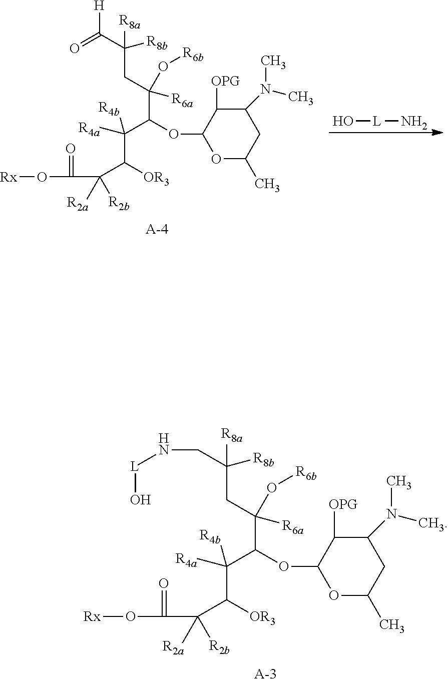

In another embodiment, the process further comprises the step of converting the compound of formula A-4A to a compound of formula A-4, by contacting a compound of formula A-4A with an oxidizing agent:

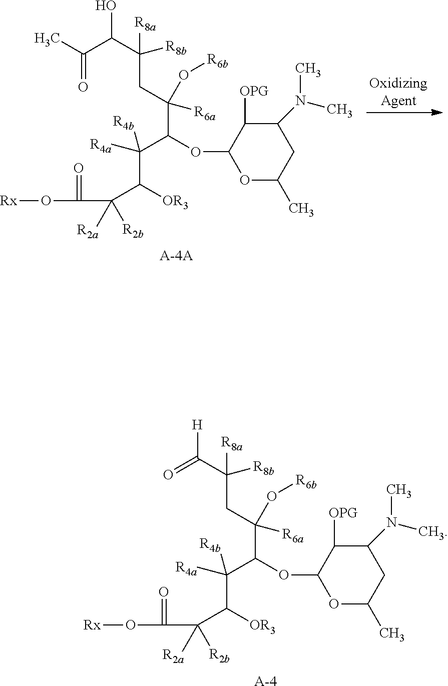

In another embodiment, the process further comprises the step of converting the compound of formula A-4A1 to a compound of formula A-4A:

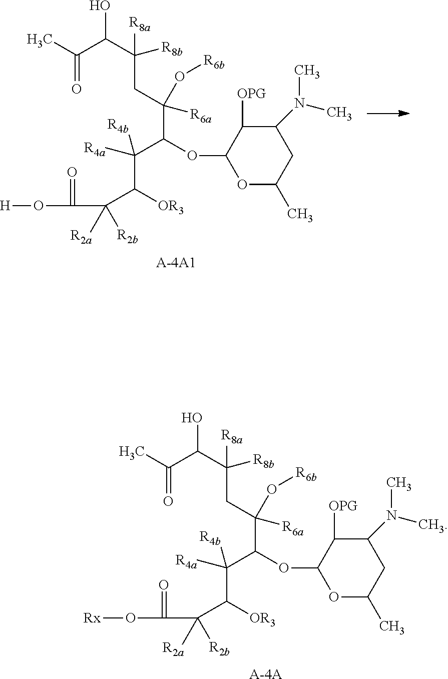

In another embodiment, the process further comprises the step of converting the compound of formula A-4A2 to a compound of formula A-4A1:

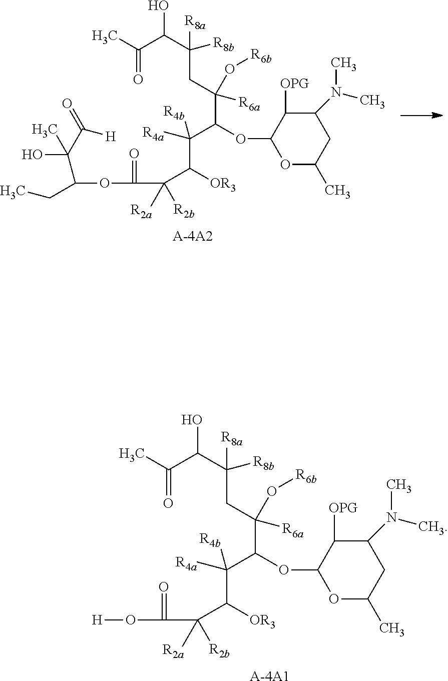

In another embodiment, the process further comprises the step of converting the compound of formula A-4A3 to a compound of formula A-4A2:

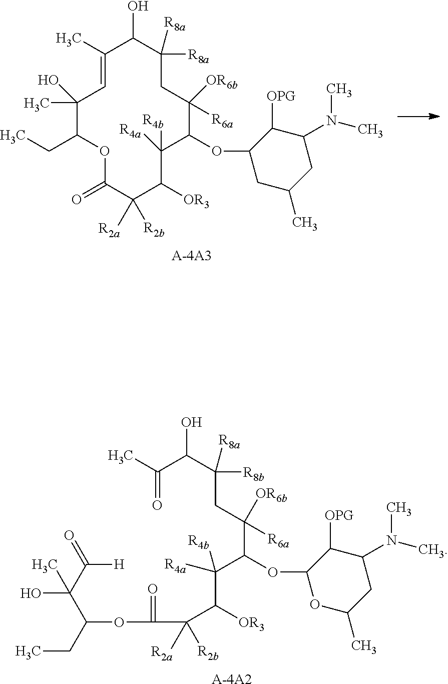

In another embodiment, the process further comprises the step of converting the compound of formula II to a compound of formula A-4A3:

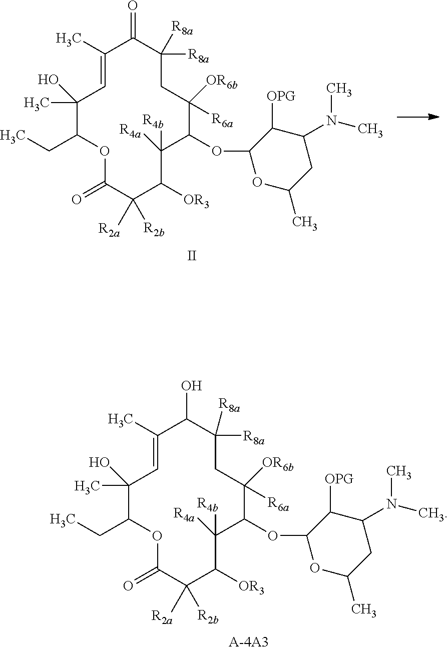

In one embodiment, the process comprises the step of converting the compound of formula III to a compound of formula II:

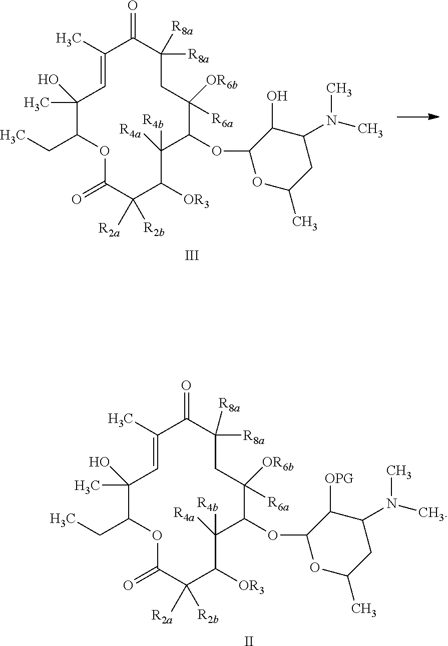

In another embodiment, the process further comprises the step of converting the compound of formula A-4B to a compound of formula A-4:

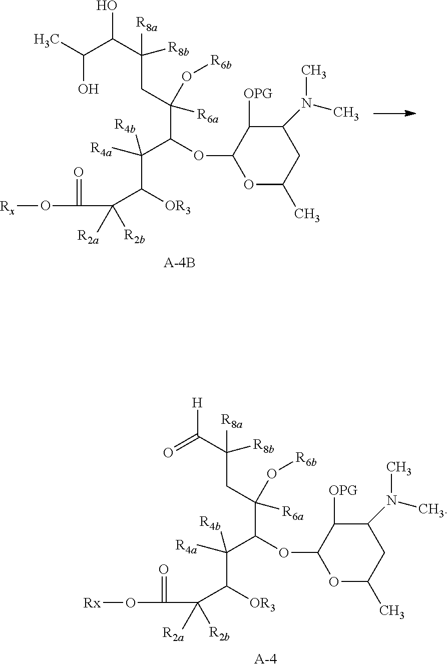

In another embodiment, the process further comprises the step of converting the compound of formula A-4B1 to a compound of formula A-4B:

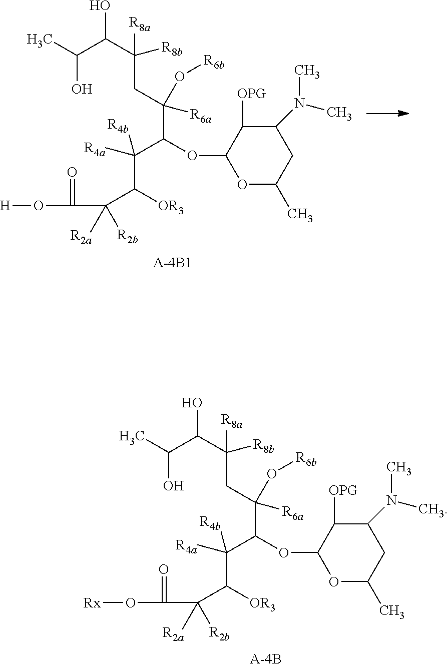

In another embodiment, the process further comprises the step of converting the compound of formula A-4B2 to a compound of formula A-4B1:

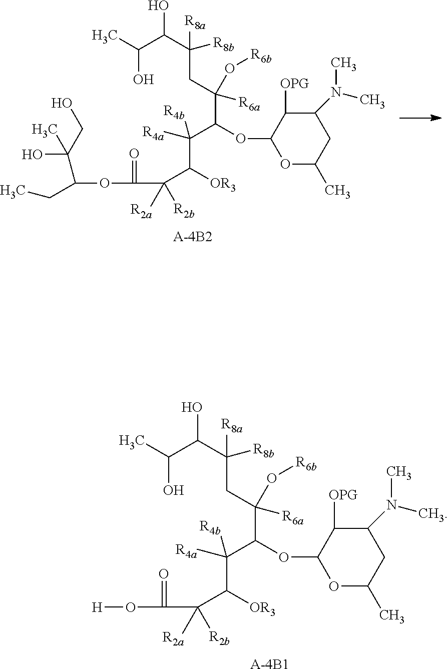

In another embodiment, the process further comprises the step of converting the compound of formula A-4B3 to a compound of formula A-4B2:

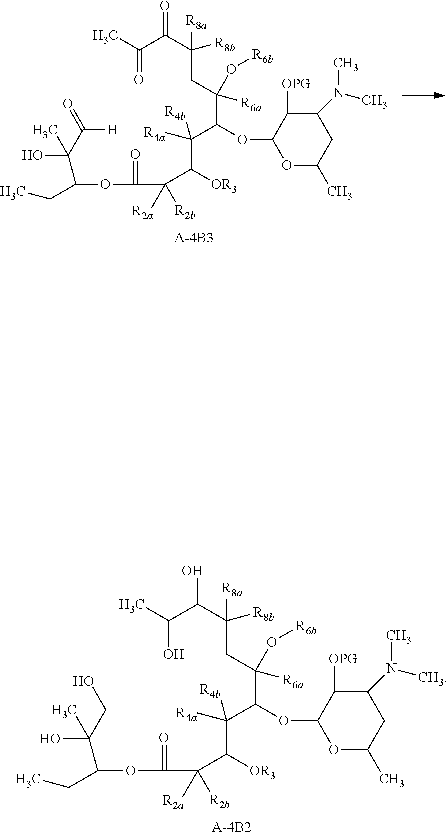

In another embodiment, the process further comprises the step of converting the compound of formula II to a compound of formula A-4B3:

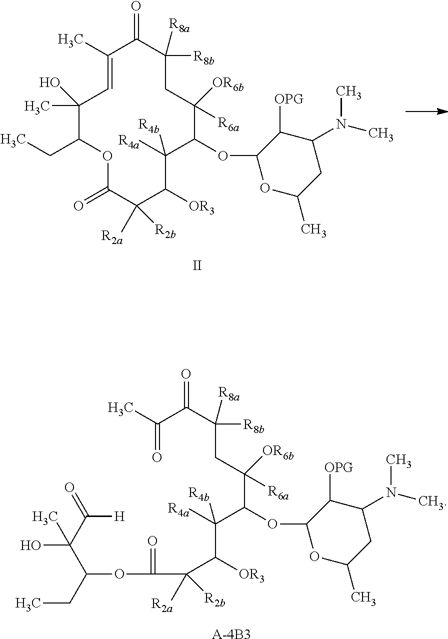

In one embodiment, R₃ is

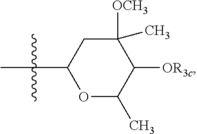

and the process further comprises the step of converting the compound of formula I to a compound of formula IA:

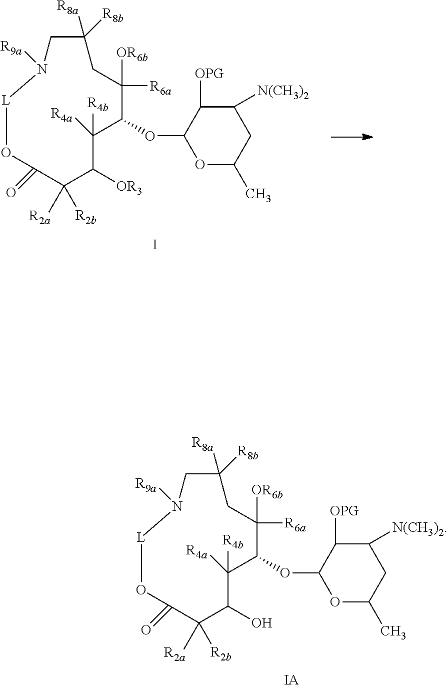

In another embodiment, the process further comprises the step of converting the compound of formula IA to a compound of formula I′:

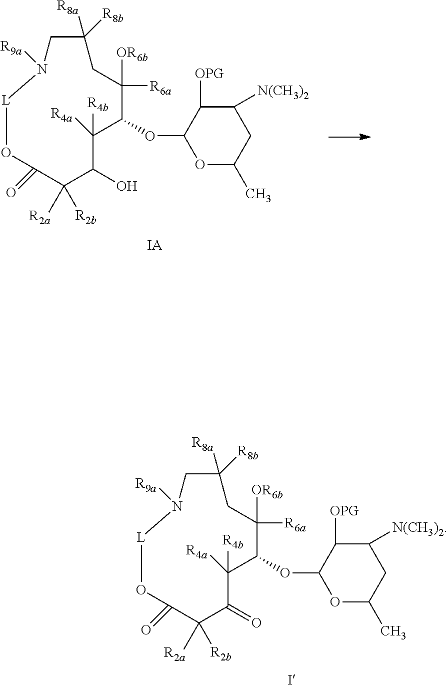

In another embodiment, the process further comprises the step of converting the compound of formula I′ to a compound of formula IC:

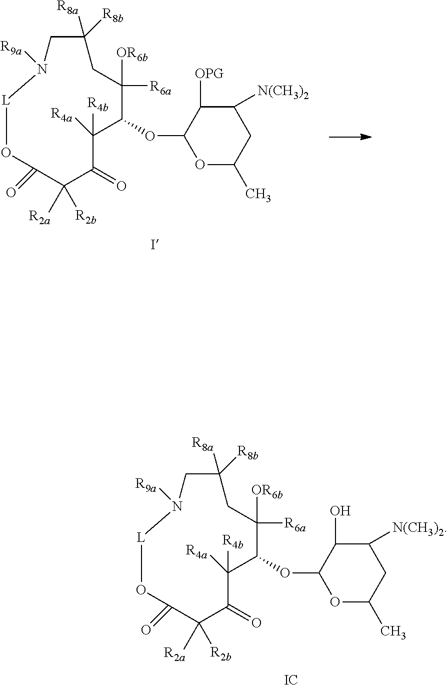

In one embodiment, the process comprises the step of:
(b1) intramolecular cyclization of a compound of formula B wherein PG is a protecting group followed by removal of the protecting group to form a compound of formula I:

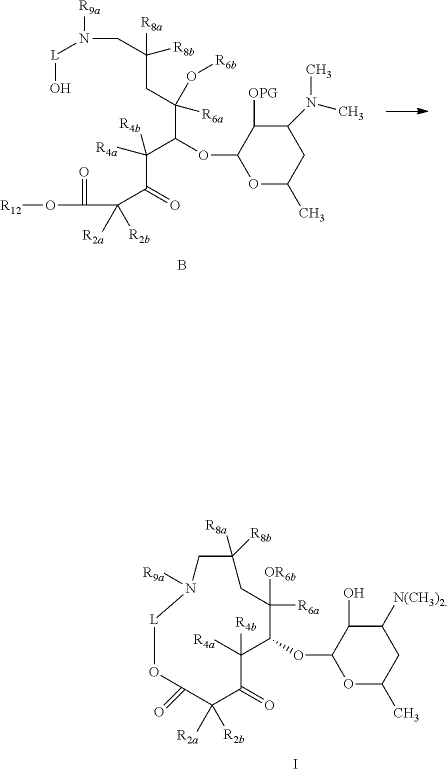

In another embodiment, the process further comprises the step of converting a compound of formula B-1 to a compound of formula B:

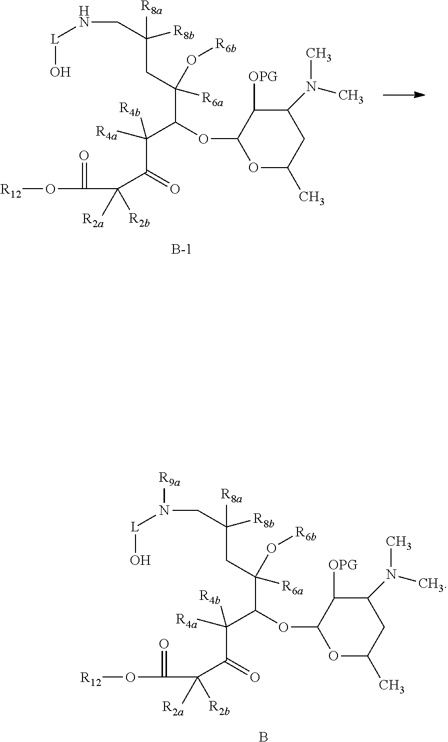

In another embodiment, the process further comprises the step of converting a compound of formula B-2 to a compound of formula B-1:

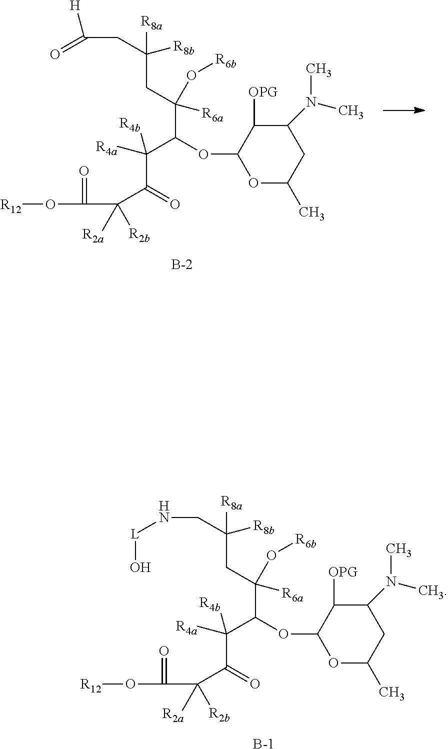

In another embodiment, the process further comprises the step of converting a compound of formula B-3 to a compound of formula B-2:

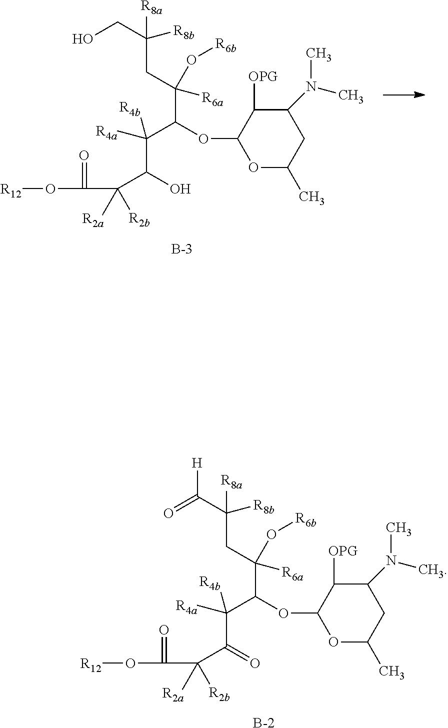

In another embodiment, the process further comprises the step of converting a compound of formula B4, wherein R₃ is

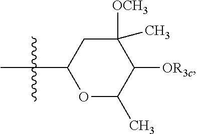

to a compound of formula B-3:

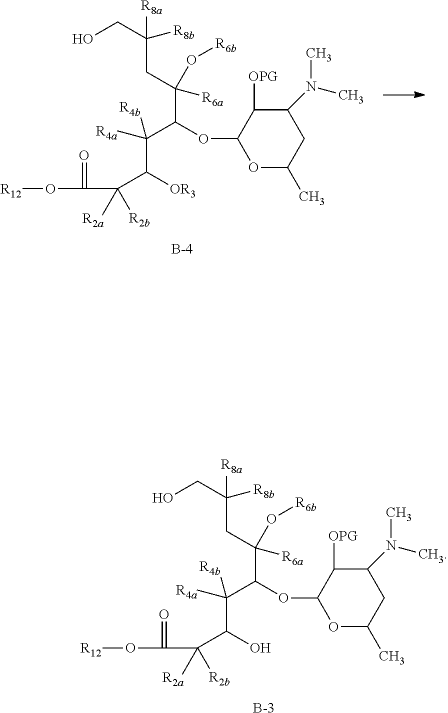

In another embodiment, the process further comprises the step of converting a compound of formula B-5 to a compound of formula B-4:

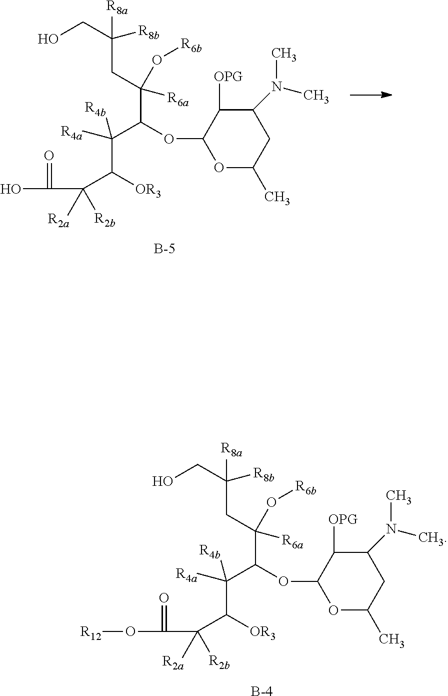

In another embodiment, the process further comprises the step of converting a compound of formula B-6 to a compound of formula B-5:

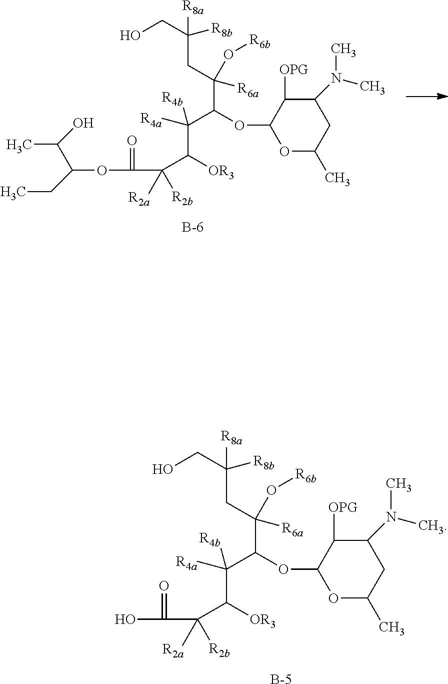

In another embodiment, the process further comprises the step of converting a compound of formula B-7 to a compound of formula B-6:

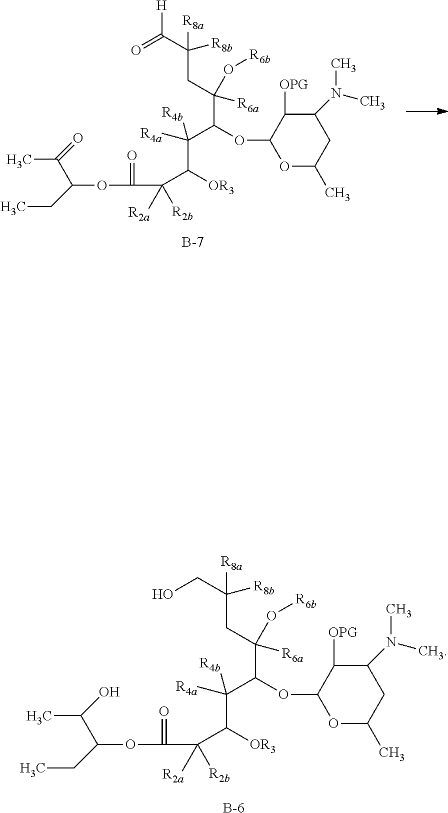

In another embodiment, the process further comprises the step of converting a compound of formula B-8 to a compound of formula B-7:

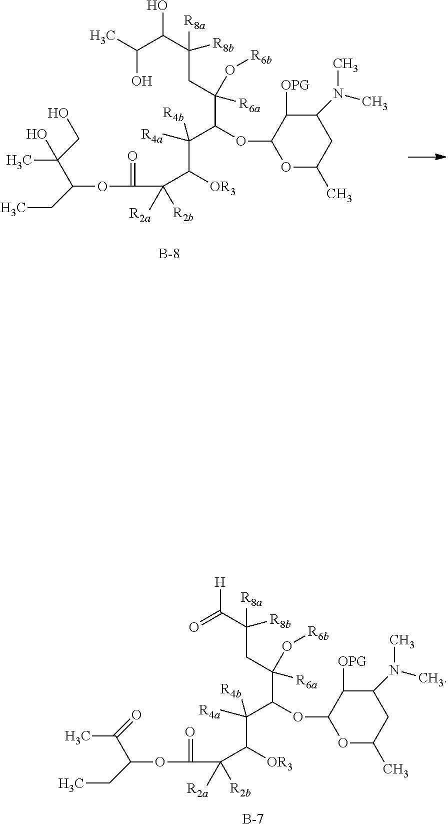

In another embodiment, the process further comprises the step of converting a compound of formula B-7 wherein R₁₀ₐ is OH and R_{10b} is H or R₁₀ₐ and R_{10b} form C═O to a compound of formula B-6:

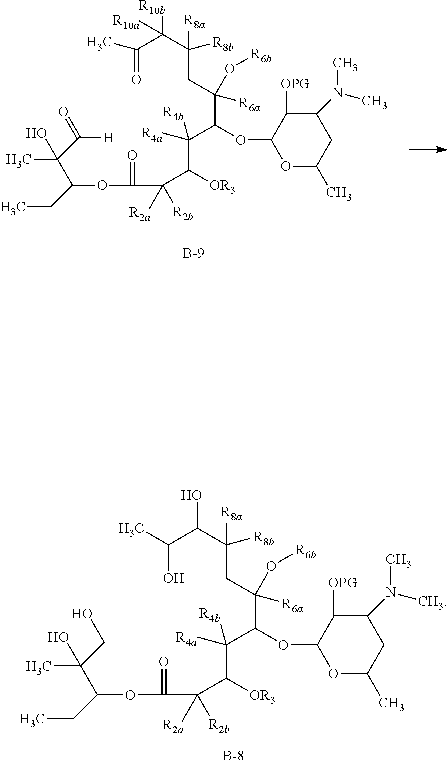

In another embodiment, the process further comprises the step of converting a compound of formula IV wherein R₁₀ₐ is OH and R_{10b} is H or R₁₀ₐ and R_{10b} form C═O to a compound of formula B-9:

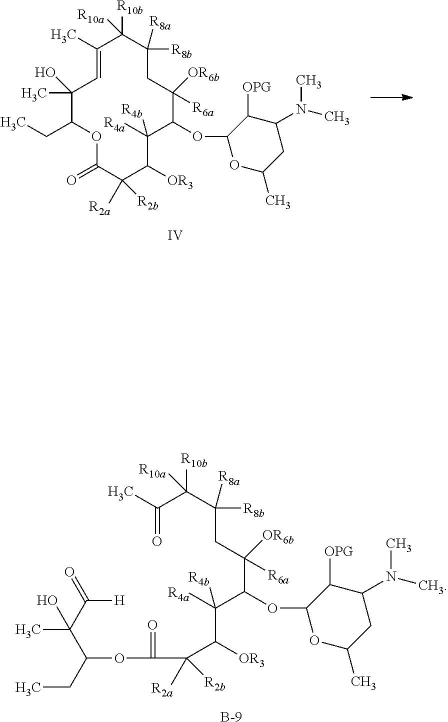

In one embodiment, R₁₂ is selected from methyl, ethyl, propyl, isopropyl, butyl, isobutyl, and t-butyl. In a further embodiment, R₁₂ is methyl or t-butyl. In still a further embodiment, R₁₂ is t-butyl.

In one embodiment, one of R₈ₐ and R_{8b} is H and the other is an optionally substituted C₁₋₁₀ alkyl.

In a further embodiment, one of R₈ₐ and R_{8b} is H and the other is methyl.

In one embodiment, R₆ₐ is selected from methyl, ethyl, propyl, isopropyl, and tert-butyl.

In one embodiment, Rab is selected from H, methyl, ethyl, propyl, isopropyl, and tert-butyl.

In a further embodiment, R₆ₐ and R_{6b} are each methyl.

In one embodiment, R₃ is H, a suitable protecting group selected from acyl, carbamoyl, alkyl ether, or silyl ether protecting groups, or

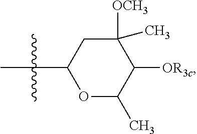

and R_{3c} is H or a benzoyl group.

In one embodiment, one of R₄ and Ras is H and the other is an optionally substituted C₁₋₁₀ alkyl.

In a further embodiment, one of R₄ₐ and R_{4b} is H and the other is methyl.

In one embodiment, one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is a C₁₋₁₀ alkyl, optionally substituted with one or more groups selected from the group consisting of halo, aryl, amino, alkyl, heteroalkyl, heteroalkenyl, heterocycloalkyl, and heteroaryl.

In another embodiment, one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is methyl.

In one embodiment, one of R₂ₐ and R_{2b} is H, and the other of R₂ₐ and R_{2b} is a C₁₋₁₀ alkyl, optionally substituted with one or more groups selected from the group consisting of halo, aryl, amino, alkyl, heteroalkyl, heteroalkenyl, heterocycloalkyl, and heteroaryl.

In another embodiment, one of R₂ₐ and R_{2b} is H, and the other of R₂ₐ and R_{2b} is methyl, ethyl, propyl, or isopropyl.

In another embodiment, one of R₂ₐ and R_{2b} is H, and the other of R₂ₐ and R_{2b} is methyl.

In one aspect, the invention includes a process for preparing a compound of formula B-9 from a compound of formula IV, comprising contacting the compound of formula IV with ozone, optionally in the presence of an acid, to form a compound of formula B-9:

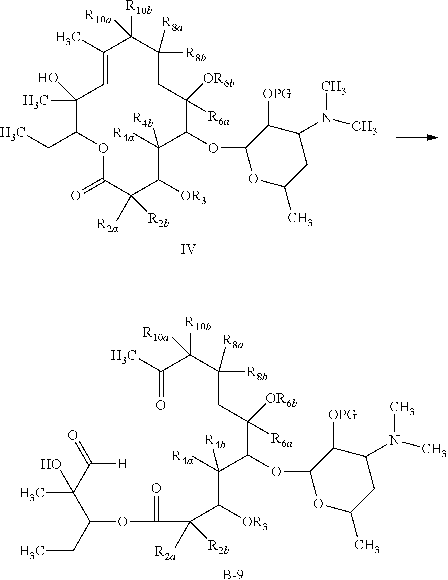

wherein:
one of R₁₀ₐ and R_{10b} is OH and the other is H or R₁₀ₐ and R_{10b} together form C═O;
one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is selected from the group consisting of halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
R₃ is H, a suitable protecting group selected from acyl, carbamoyl, alkyl ether, or silyl ether protecting groups, or

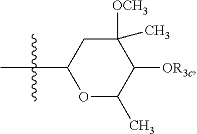

wherein R_{3c} is H or a protecting group;
each of R₄ₐ and R_{4b} is independently selected from the group consisting of —H, and optionally substituted C₁₋₁₀ alkyl;
R_{6b} is optionally substituted C₁₋₁₀ alkyl;
R_{6b} is —H, optionally substituted C₁₋₁₀ alkyl, optionally substituted C₁₋₁₀ hydroxyalkyl, and optionally substituted allyl;
R₈ₐ and R_{8b} are each independently selected from the group consisting of —H and optionally substituted C₁₋₁₀ alkyl; and
PG is a suitable protecting group.

In one embodiment, the compound of formula IV is contacted with ozone in the presence of an acid to form a compound of formula B-9. In a further embodiment, the acid is soluble in dichloromethane.

In still a further embodiment, the acid is trifluoroacetic acid.

In one embodiment, the contacting occurs in dichloromethane.

In another embodiment, one of R₈ₐ and R_{8b} is H and the other is an optionally substituted C₁₋₁₀alkyl.

In a further embodiment, one of R₈ₐ and R_{8b} is H and the other is methyl.

In one embodiment, R₆ₐ is selected from methyl, ethyl, propyl, isopropyl, and tert-butyl.

In one embodiment, R_{6b} is selected from H, methyl, ethyl, propyl, isopropyl, and tert-butyl.

In a further embodiment, R₆ₐ and R_{6b} are each methyl.

In one embodiment, R₃ is H, a suitable protecting group selected from acyl, carbamoyl, alkyl ether, or silyl ether protecting groups, or

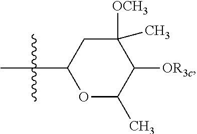

and R_{3c} is H or a benzoyl group

In one embodiment, one of R₄ₐ and R_{4b} is H and the other is an optionally substituted C₁₋₁₀ alkyl.

In a further embodiment, one of R₄ₐ and R_{4b} is H and the other is methyl.

In one embodiment, one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is a C₁₋₁₀ alkyl, optionally substituted with one or more groups selected from the group consisting of halo, aryl, amino, alkyl, heteroalkyl, heteroalkenyl, heterocycloalkyl, and heteroaryl.

In one embodiment, one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is methyl.

In one embodiment, one of R₂ₐ and R_{2b} is H, and the other of R₂ₐ and R_{2b} is a C₁₋₁₀ alkyl, optionally substituted with one or more groups selected from the group consisting of halo, aryl, amino, alkyl, heteroalkyl, heteroalkenyl, heterocycloalkyl, and heteroaryl.

In another embodiment, one of R₂ₐ and R_{2b} is H, and the other of R₂ₐ and R_{2b} is methyl, ethyl, propyl, or isopropyl.

In a further embodiment, one of R₂ₐ and R_{2b} is H and the other of R₂ₐ and R_{2b} is methyl.

In one aspect, the invention includes a process for preparing a compound of formula A-3 from a compound of formula A-4, comprising coupling the compound of formula A-4 with an amine having the formula NH₂-L-OH under reductive amination conditions to form a compound of formula A-3:

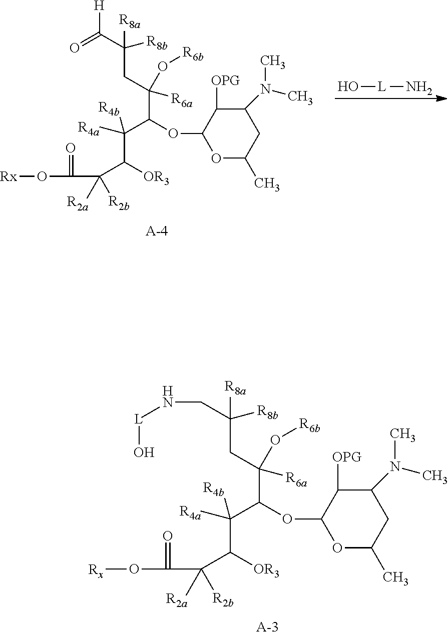

wherein:
one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is selected from the group consisting of halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
R₃ is H, a suitable protecting group selected from acyl, carbamoyl, alkyl ether, or silyl ether protecting groups, or

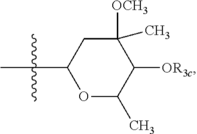

wherein R_{3c} is H or a protecting group;
each of R₄ₐ and R_{4b} is independently selected from the group consisting of —H, and optionally substituted C₁₋₁₀alkyl;
R₆ₐ is optionally substituted C₁₋₁₀ alkyl;
R_{6b} is —H, optionally substituted C₁₋₁₀ alkyl, optionally substituted C₁₋₁₀ hydroxyalkyl, and optionally substituted allyl;
R₈ₐ and R_{8b} are each independently selected from the group consisting of —H and optionally substituted C₁₋₁₀alkyl;
PG is a suitable protecting group; and
L is an optionally substituted C₂₋₅ alkylene.

In one embodiment, the coupling the compound of formula A-4 with an amine having the formula NH₂-L-OH is performed in the presence of a solvent.

In a further embodiment, the solvent is dichloromethane.

In one embodiment, the coupling the compound of formula A-4 with an amine having the formula NH₂-L-OH is performed in the presence of NaB(OAc)₃H and acetic acid.

In one embodiment, the NaB(OAc)₃H and acetic acid are each present in about 2 molar equivalents with respect to the compound of formula A-4.

In some embodiments, L is a C₂₋₈ optionally substituted alkylene. In some embodiments, L is a C₂₋₆ optionally substituted alkylene. In some embodiments, L is a C₂₋₅ optionally substituted alkylene. In some embodiments, L is a C₂₋₄ optionally substituted alkylene. In one embodiment, L is ethylene, which is optionally substituted with up to three of halo, CN, NO₂, amino, amido, carboxy, alkylcarbonyl, alkoxycarbonyl, alkyl, alkoxy, cycloalkyl, heterocycloalkyl, aryl, or heteroaryl, wherein the alkyl, alkoxy, cycloalkyl, heterocycloalkyl, aryl, and heteroaryl are each independently and optionally substituted with halo or alkyl.

In one embodiment, L is ethylene, which is optionally substituted with up to three of halo, alkyl, or alkoxy.

In one embodiment, NH₂-L-OH is NH₂—CH₂-L₁-OH, NH₂—CH(C₁₋₆ alkyl)-L₁OH, NH₂-L₁-CH₂—OH or NH₂-L₁-CH(C₁₋₆ alkyl)-OH. In one embodiment, NH₂-L-OH is NH₂-L₁-CH₂—OH.

In one embodiment, L₁ is CH—R₁₄ₐ, wherein R₁₄ₐ is selected from the group consisting of an optionally substituted R₁₀₁—CH₂—, R₁₀₁—CH₂CH₂—, R₁₀₁—CH₂CH₂CH₂—, optionally substituted R₁₀₁—CH₂CH₂CH—OH—, and optionally substituted R₁₀₁—CH₂CH₂CH—OMe-. In this embodiment, R₁₀₁ is selected from the group consisting of

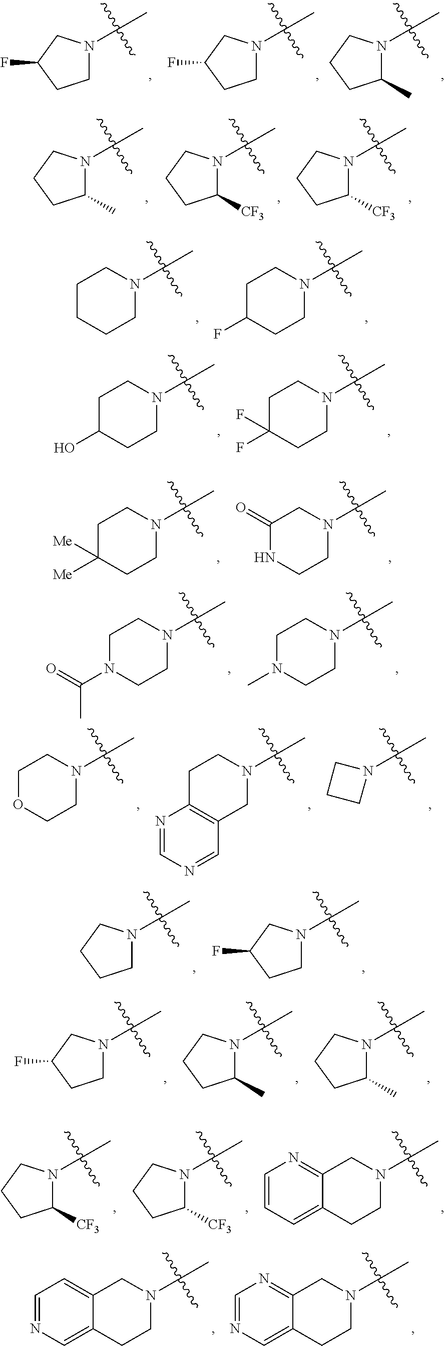

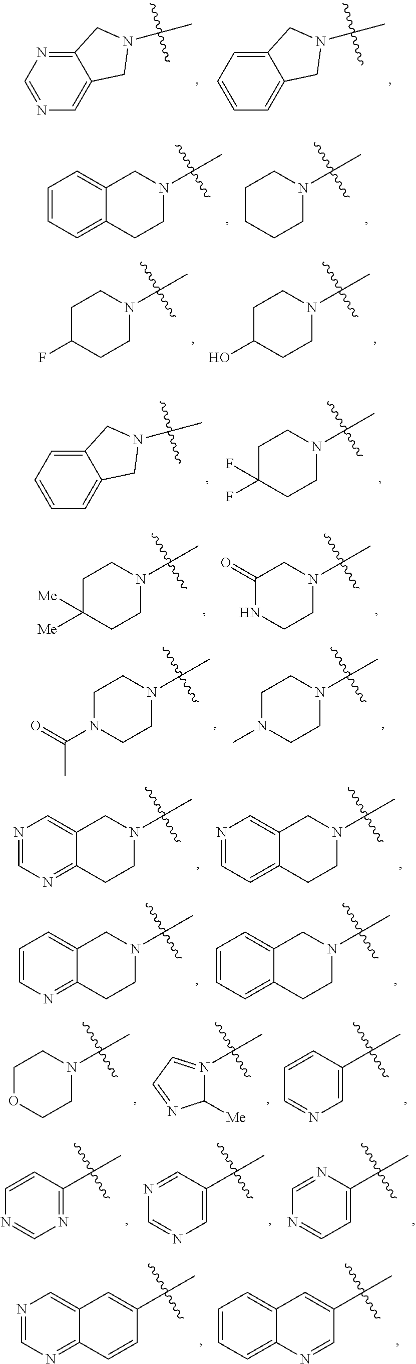

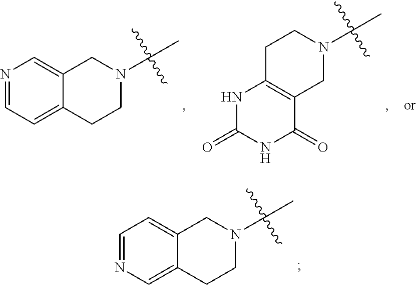 [SMILES: *N1CCc2ccncc2C1 ; *N1CCc2[nH]c(=O)[nH]c(=O)c2C1 ; *N1CCc2cnccc2C1]

where [symbol] indicates a point of attachment.

In one embodiment, L₁ is CH—R₁₄ₐ, wherein R₁₄ₐ is optionally substituted saturated or partially unsaturated cycloalkyl containing at least one double bond, optionally substituted saturated or partially unsaturated heterocycloalkyl, optionally substituted aryl, and optionally substituted heteroaryl. In one embodiment, R₁₄ₐ is

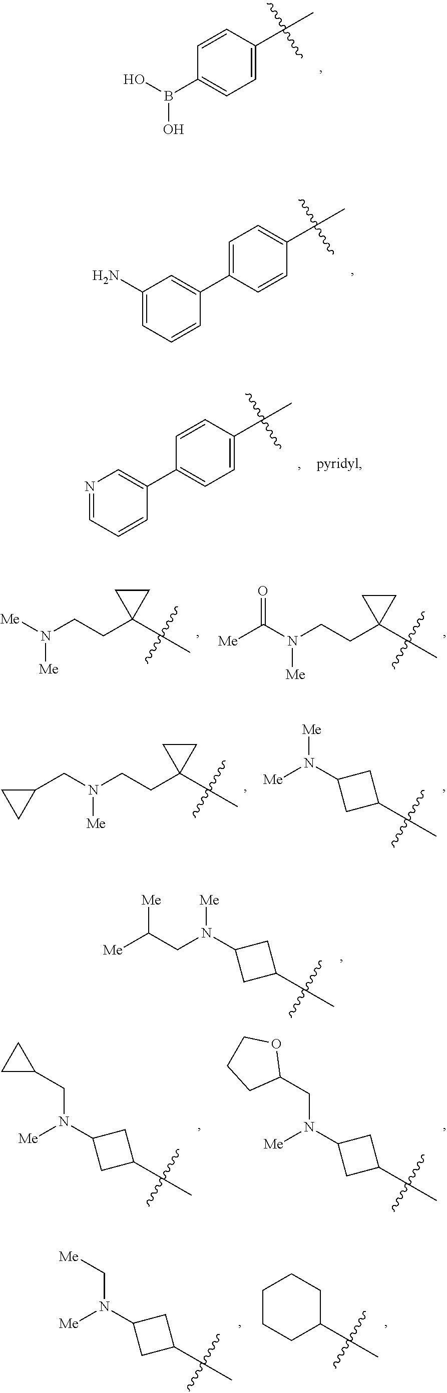

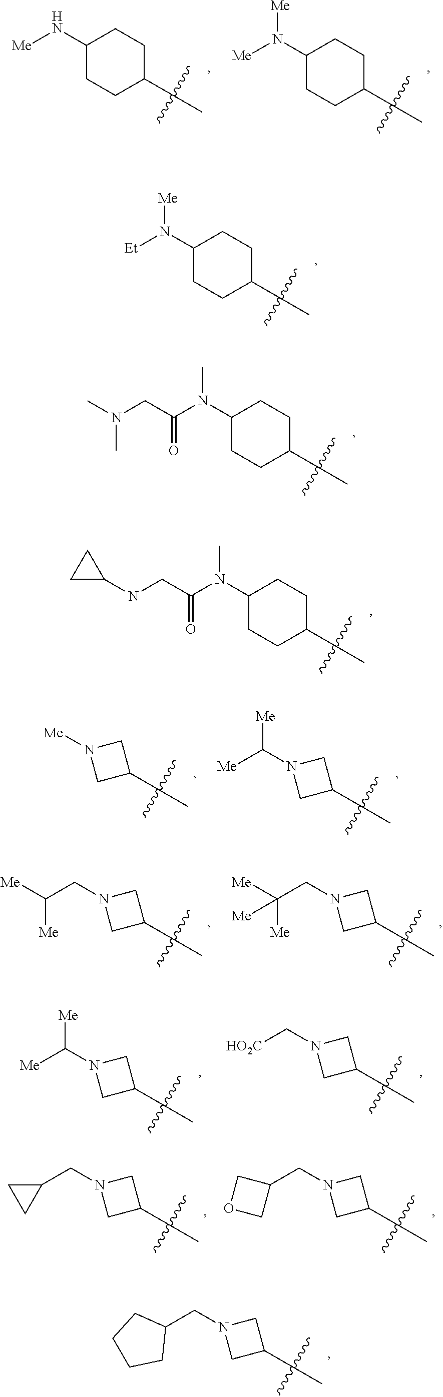

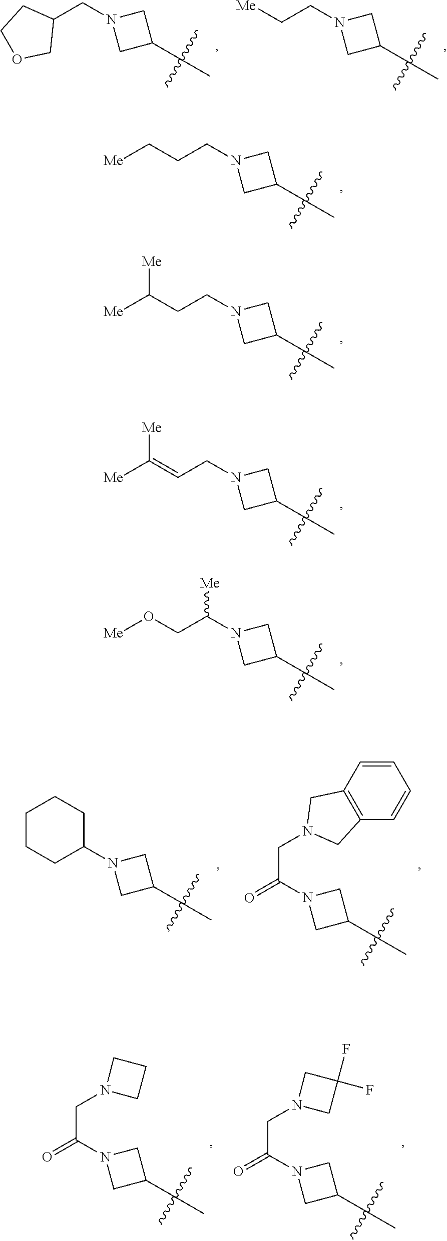

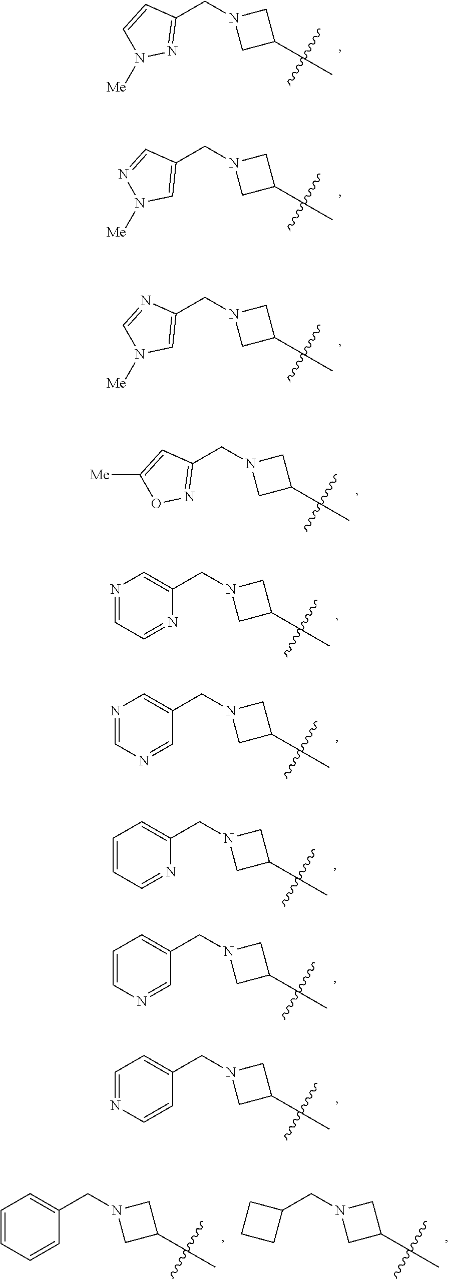

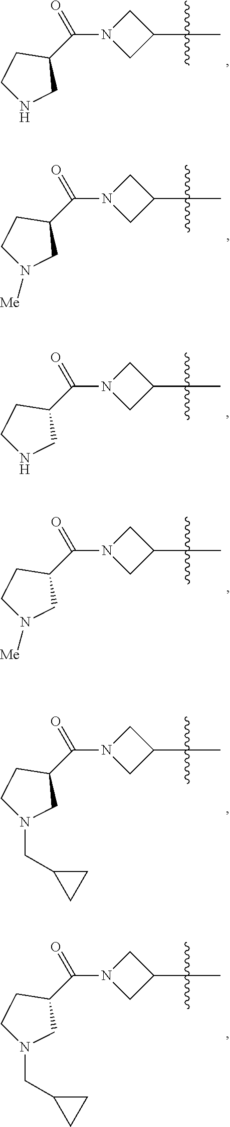 [SMILES: *C1CN(C(=O)[C@@H]2CCNC2)C1 ; *C1CN(C(=O)[C@@H]2CCN(C)C2)C1 ; *C1CN(C(=O)[C@H]2CCNC2)C1 ; *C1CN(C(=O)[C@H]2CCN(C)C2)C1 ; *C1CN(C(=O)[C@@H]2CCN(CC3CC3)C2)C1 ; *C1CN(C(=O)[C@H]2CCN(CC3CC3)C2)C1]

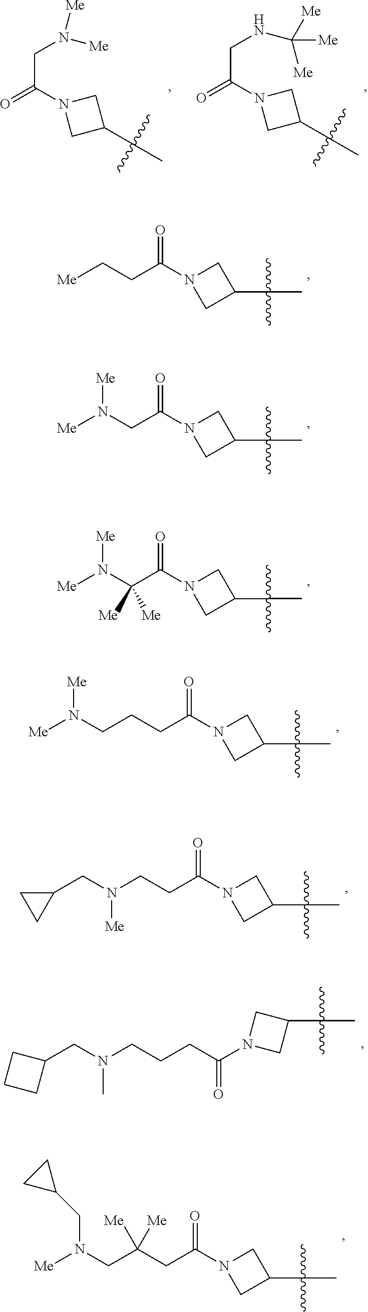

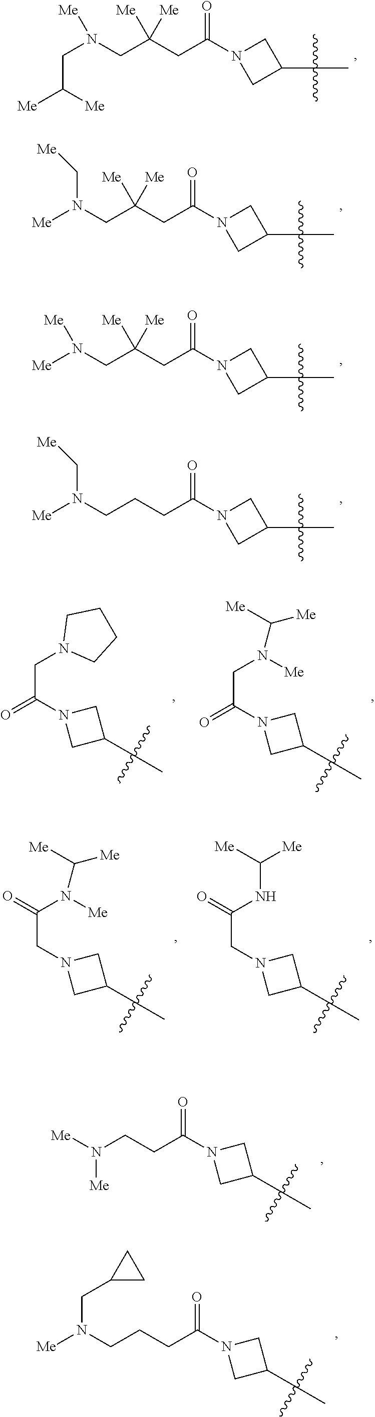

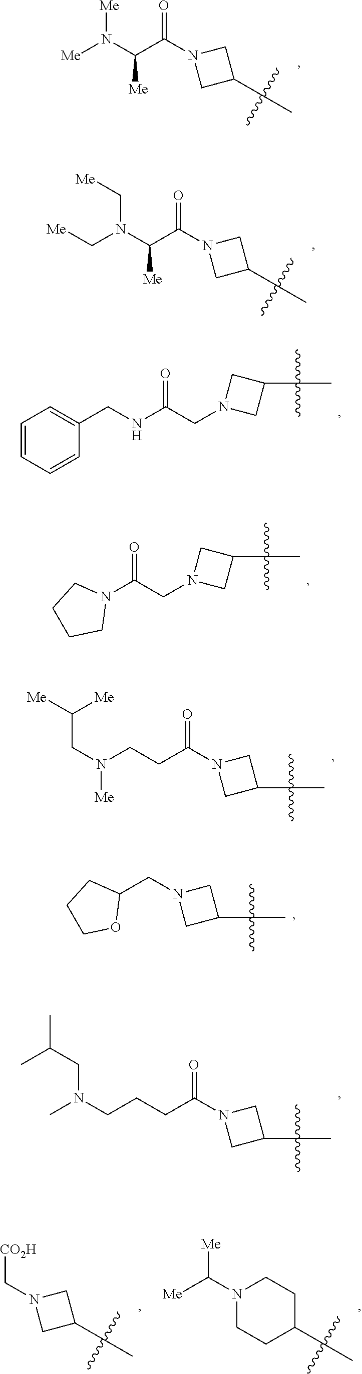

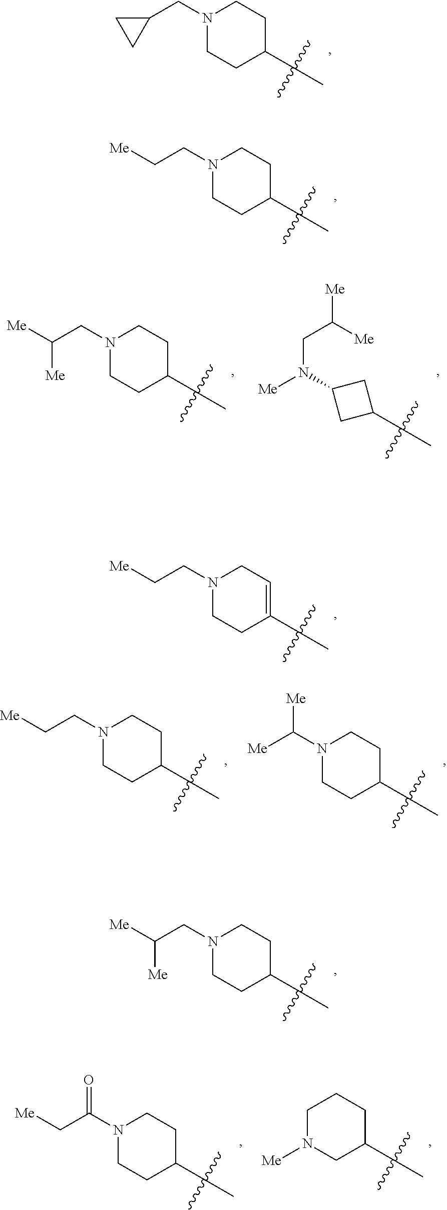

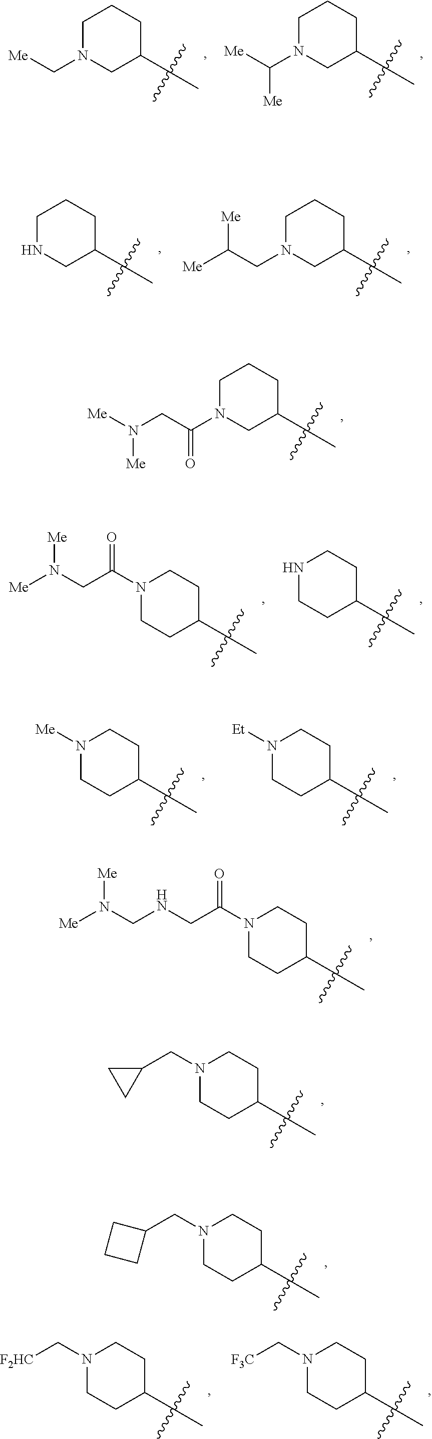

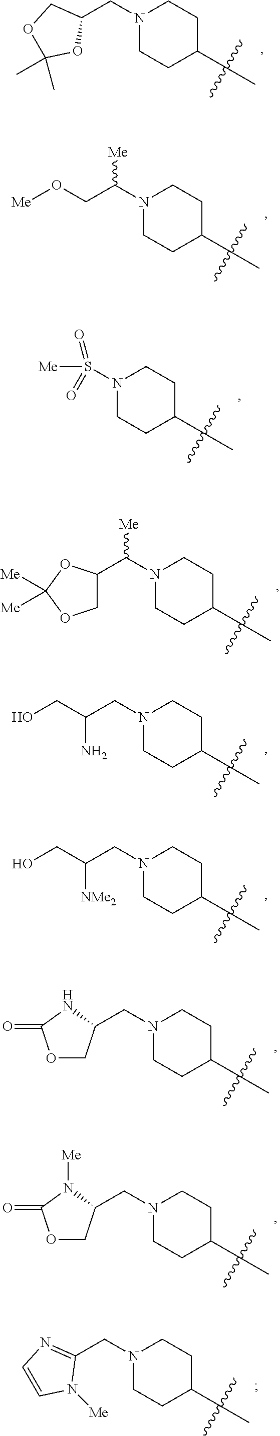

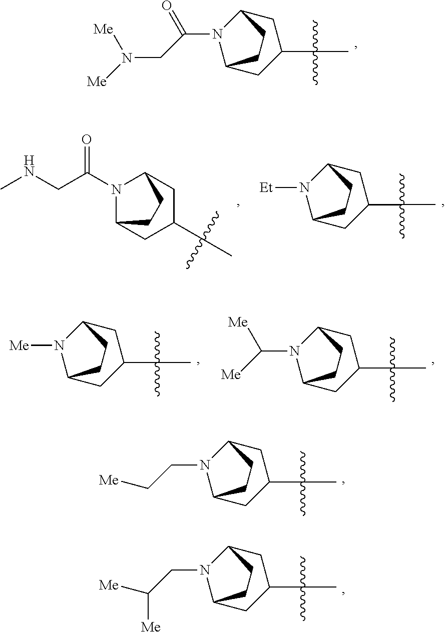

wherein “ [symbol] ” indicates a point of attachment.

In one embodiment, L₁ is CR₁₄ₐR_{14b}, wherein R₁₄ₐ and R_{14b} are taken together with the carbon atom to which they are attached to form

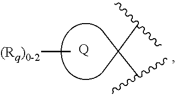

wherein Q is a 3-, 4-, 5-, or 6-membered cycloalkyl or heterocyclic ring, wherein “ [symbol] ” indicates points of attachment and R₁₁ₐ and R_{11b} are each independently selected from the group consisting of H, halo, and optionally substituted C₁₋₁₀ alkyl. In one embodiment, each R_{q} is independently selected from the group consisting of H, —CH₂Cl, carboxybenzyl, acetyl, methyl, ethyl, propyl, isopropyl, butyl, isobutyl, tertbutyl, benzyl,

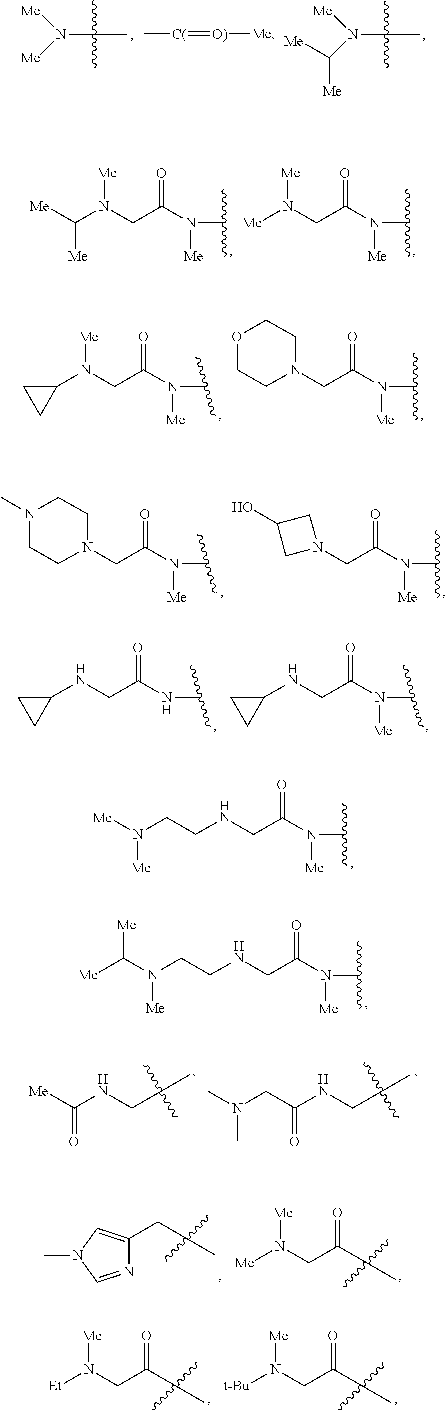

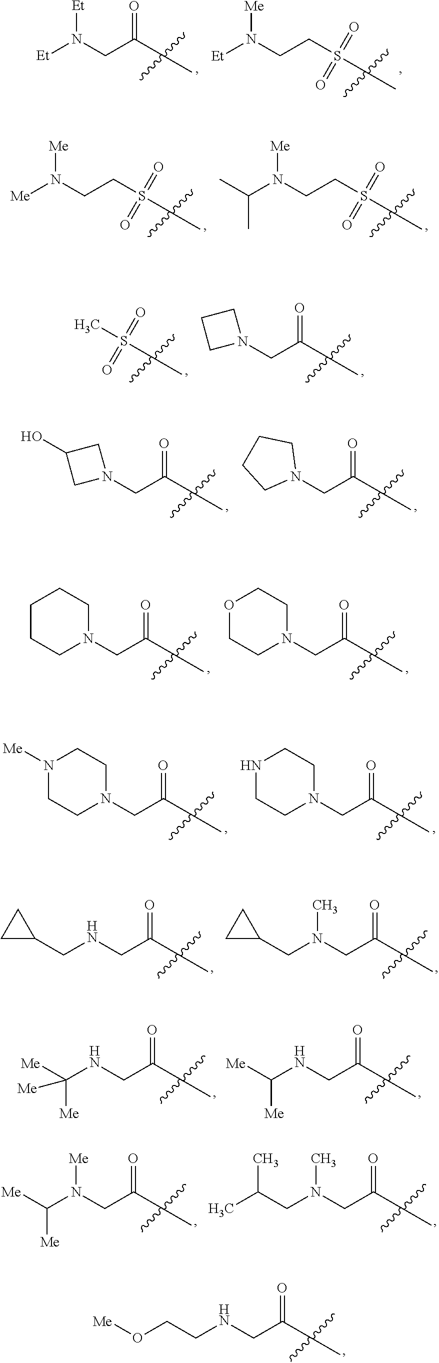

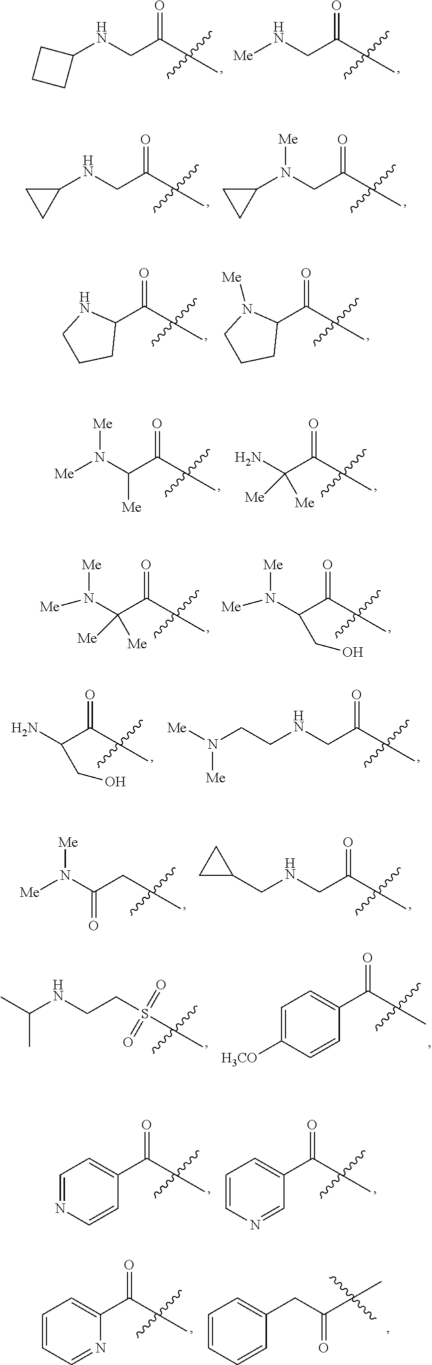

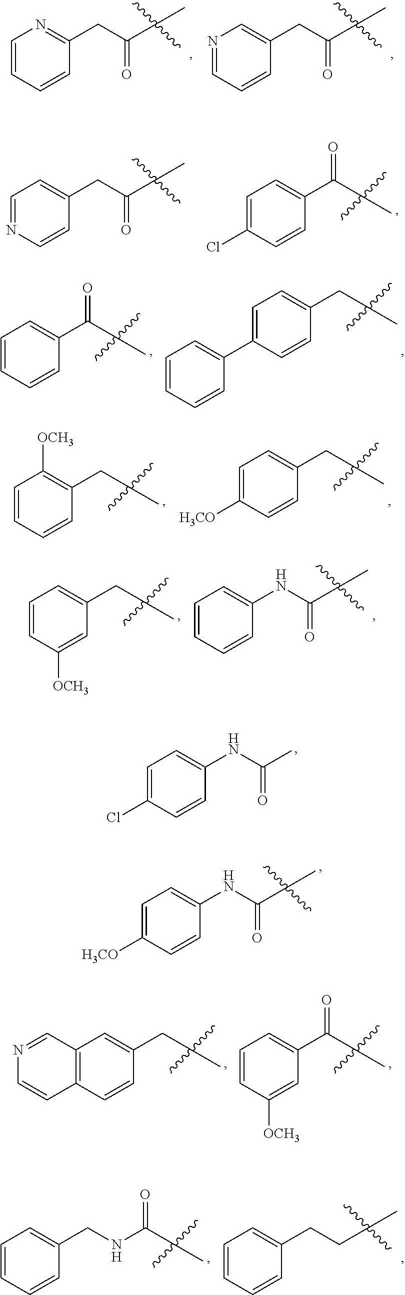

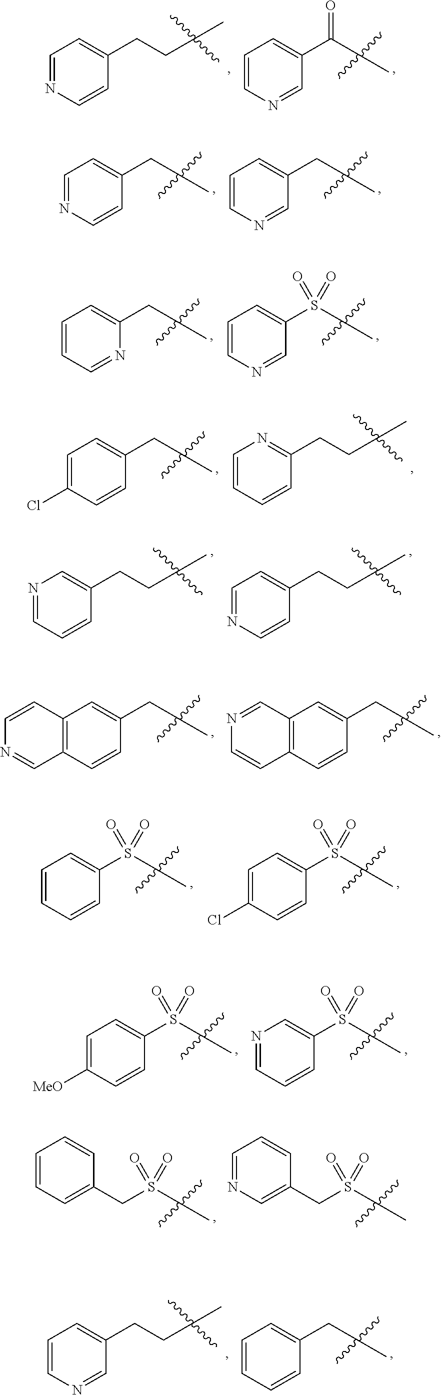

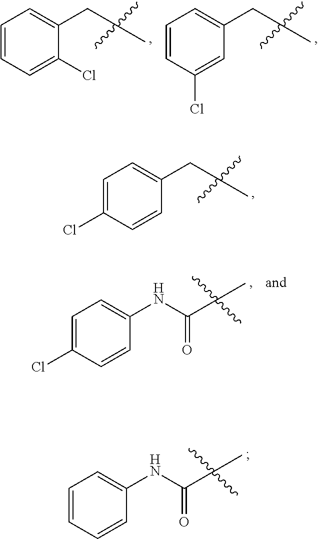 [SMILES: *Cc1ccccc1Cl ; *Cc1cccc(Cl)c1 ; *Cc1ccc(Cl)cc1 ; *C(=O)Nc1ccc(Cl)cc1 ; *C(=O)Nc1ccccc1]

or two R_{q} together with the atom to which they are attached form oxo,

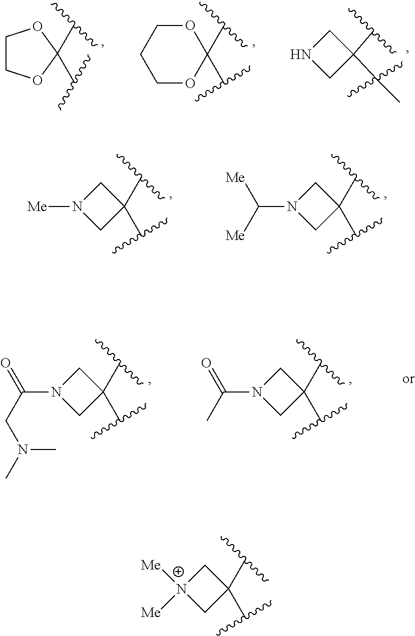

(as a salt) (e.g., the formate salt), wherein “ [symbol] ” indicates a point of attachment.

In one embodiment, of the compound of formula I, IA, IB, IC, ID, IE, IF, and IG,

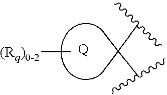

is

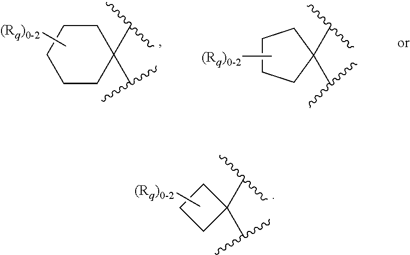

In one embodiment,

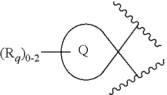

is

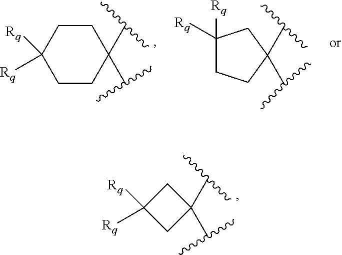

and each R_{q} is independently selected from the group consisting of H, —CH₂Cl, carboxybenzyl, acetyl, methyl, ethyl, propyl, isopropyl, butyl, isobutyl, tertbutyl, benzyl,

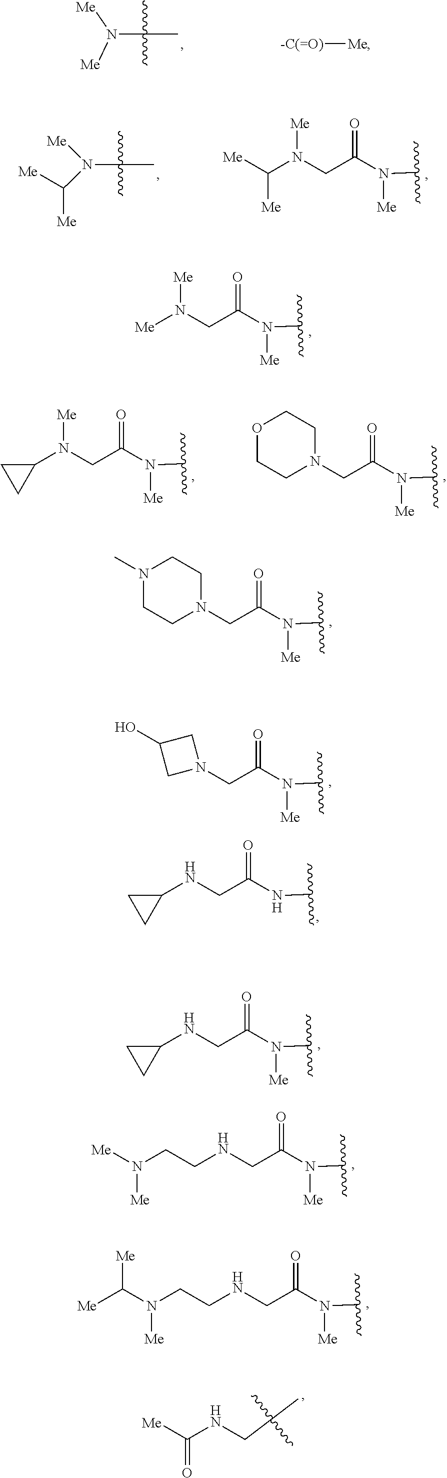

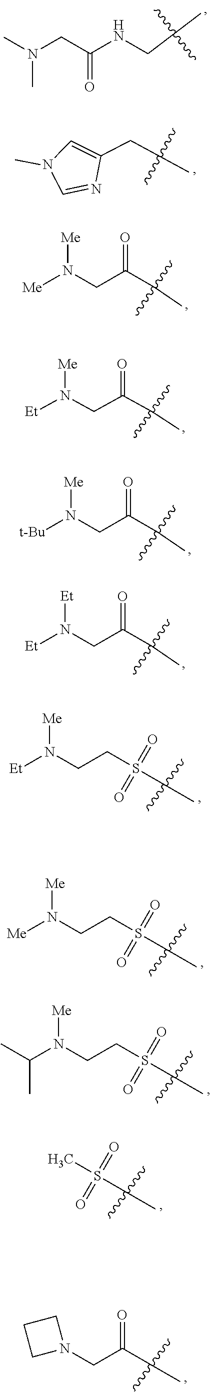

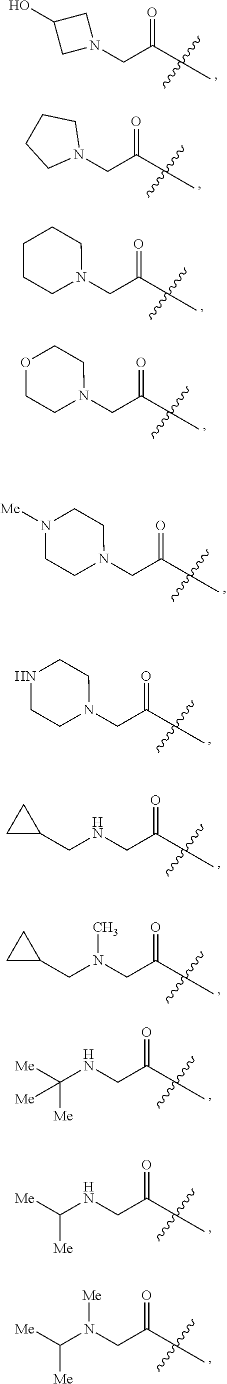

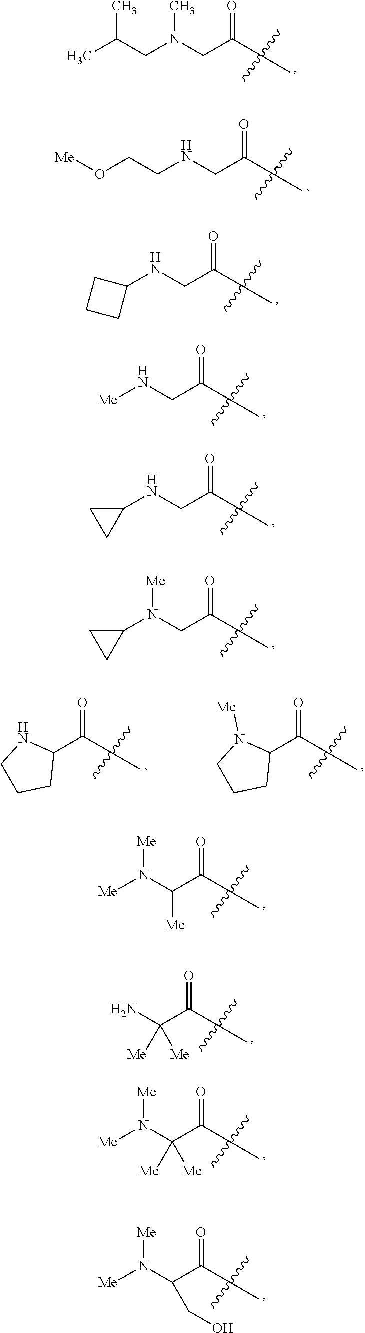

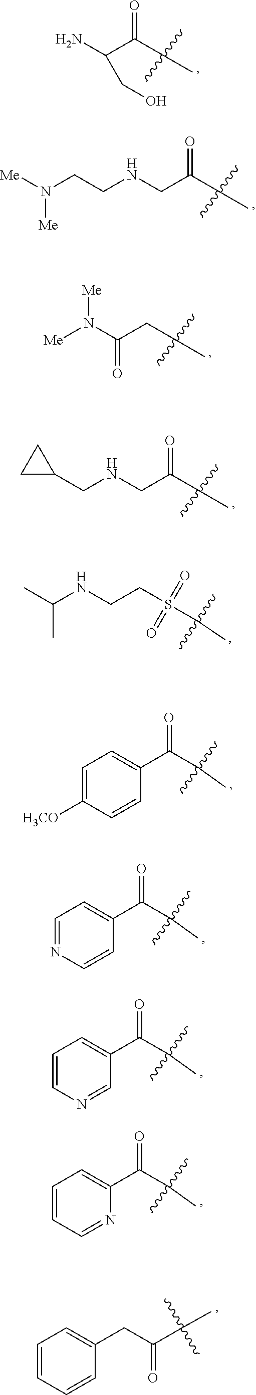

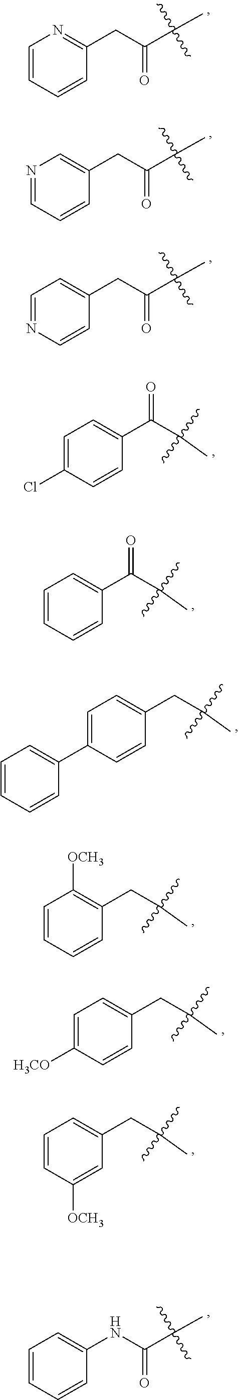

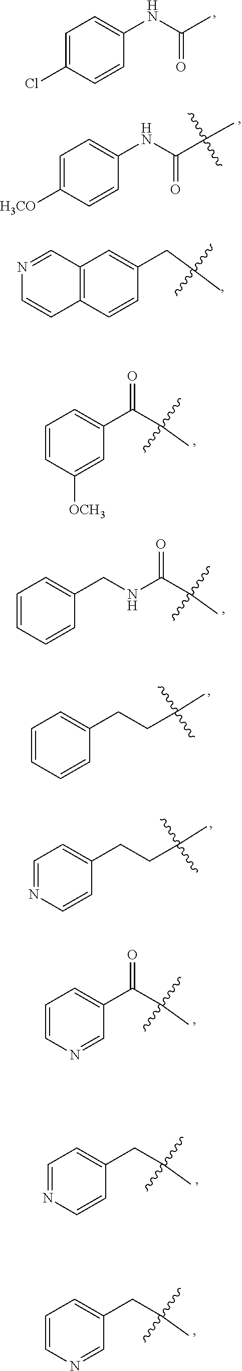

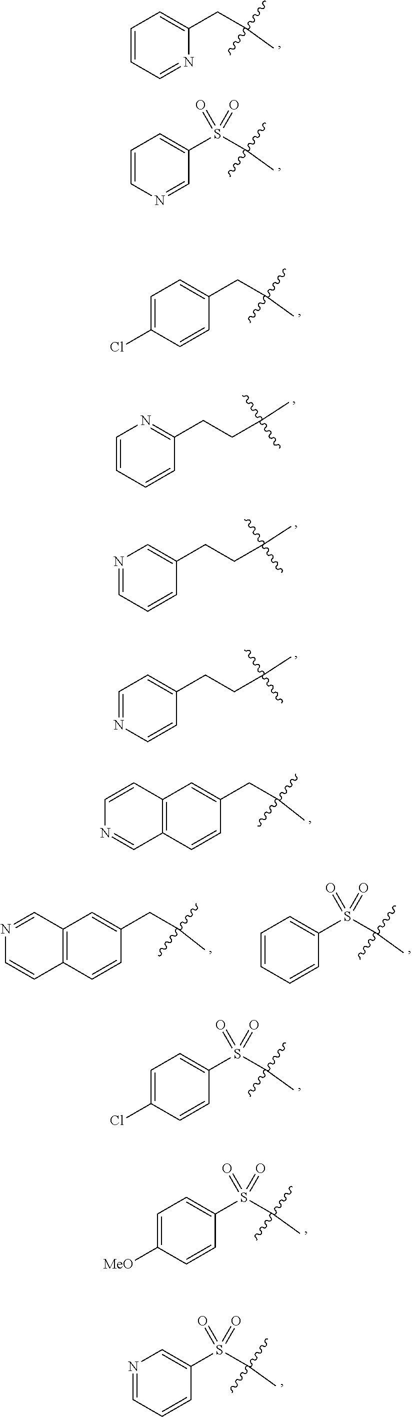

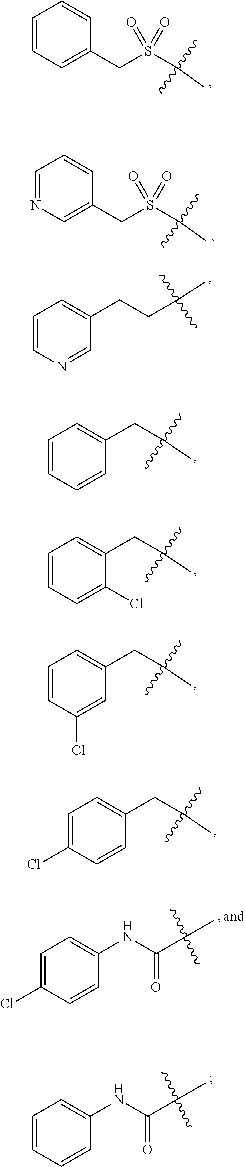

or two R_{q} together with the atom to which they are attached form oxo,

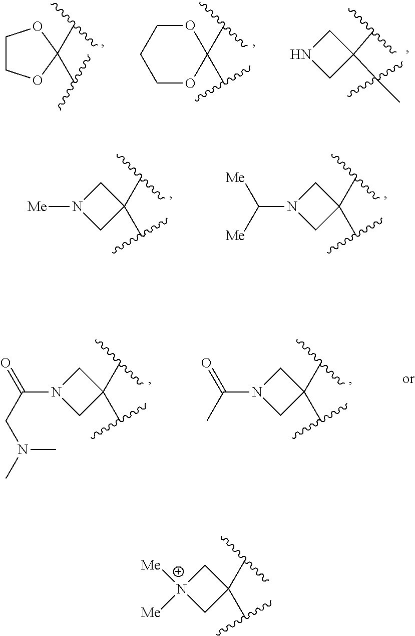

(as a salt)(e.g., the formate salt), wherein “ [symbol] ” indicates a point of attachment.

In one embodiment, of the compound of formula I, IA, IB, IC, ID, IE, IF, and IG,

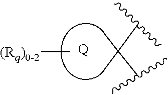

is

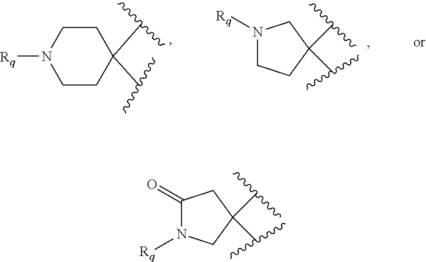

and R_{q} is selected from the group consisting of H, —CH₂Cl, carboxybenzyl, acetyl, methyl, ethyl, propyl, isopropyl, butyl, isobutyl, tertbutyl, benzyl, —C(═O)-Me,

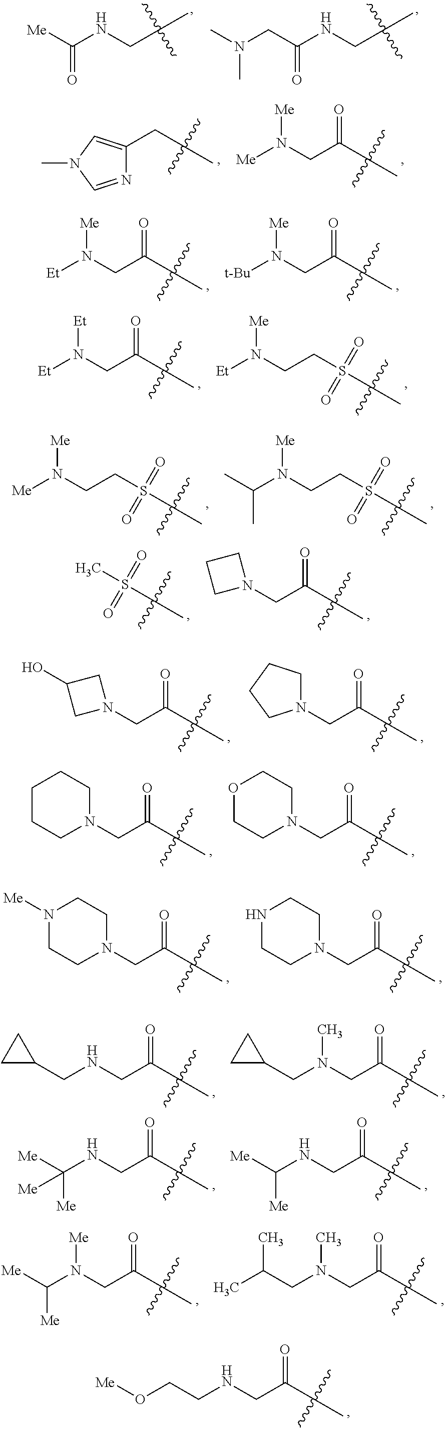

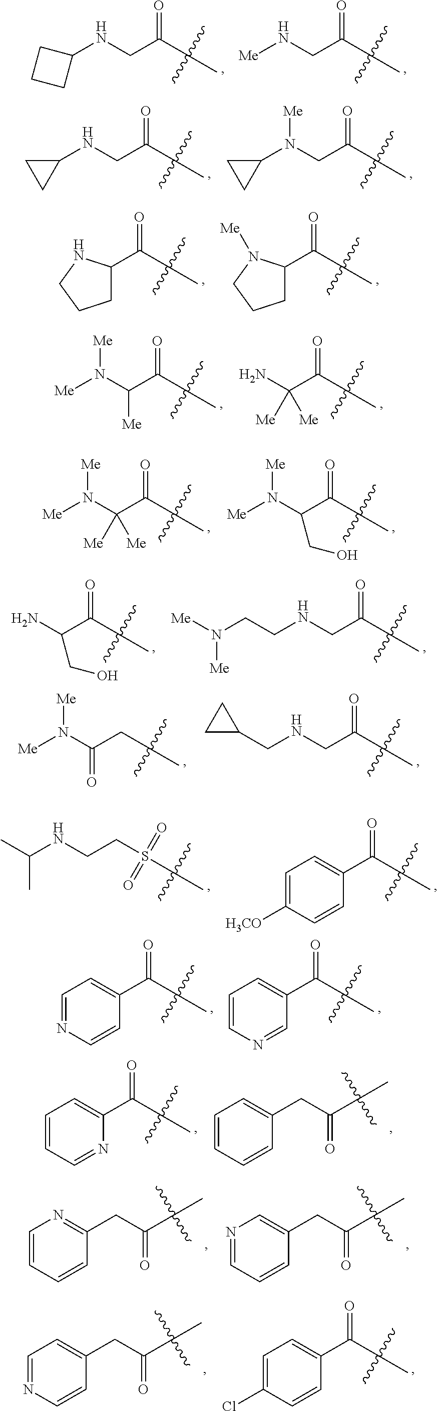

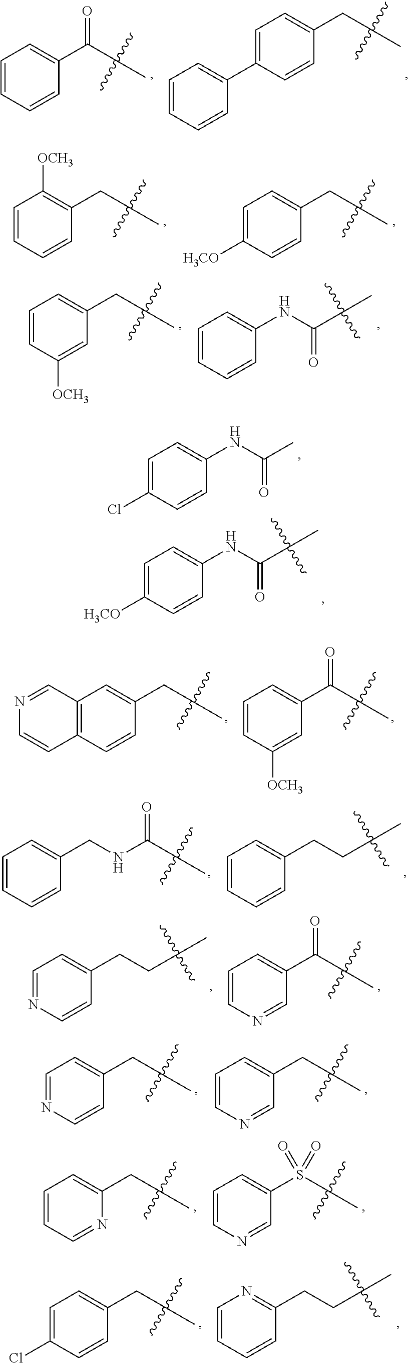

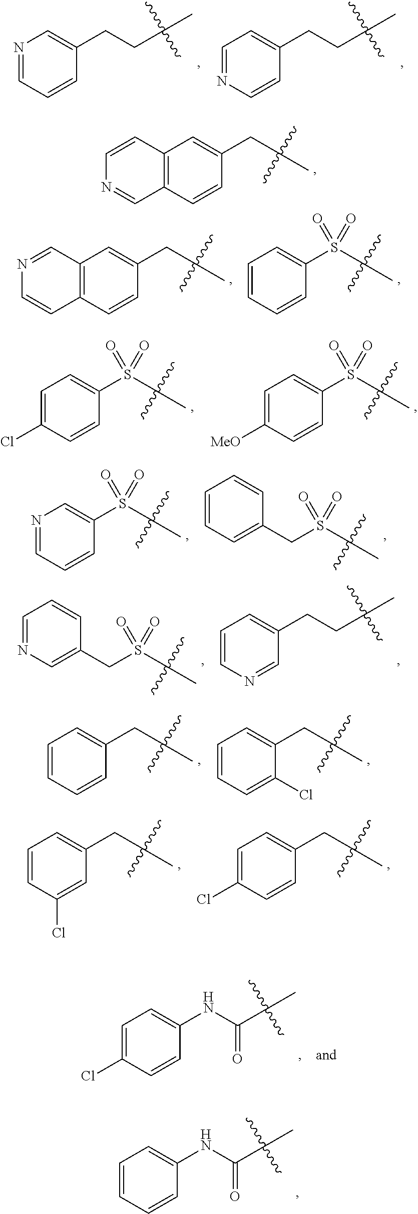

wherein “ [symbol] ” indicates a point of attachment.

In one embodiment, one of R₈ₐ and R_{8b} is H and the other is an optionally substituted C₁₋₁₀ alkyl.

In a further embodiment, one of R₈ₐ and R_{8b} is H and the other is methyl.

In one embodiment, R₆ₐ is selected from methyl, ethyl, propyl, isopropyl, and tert-butyl.

In one embodiment, R_{6b} is selected from H, methyl, ethyl, propyl, isopropyl, and tert-butyl.

In a further embodiment, R₆ₐ and R_{6b} are each methyl.

In one embodiment, R_{3c} is a protecting group selected from benzoyl, p-nitrobenzoyl, TMS, TES IPDMS, TBS, or methoxymethyl. In a further embodiment, R_{3c} is a benzoyl group.

In one embodiment, one of R₄ₐ and R_{4b} is H and the other is an optionally substituted C₁₋₁₀ alkyl.

In a further embodiment, one of R₄ₐ and R_{4b} is H and the other is methyl.

In one embodiment, one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is a C₁₋₁₀ alkyl, optionally substituted with one or more groups selected from the group consisting of halo, aryl, amino, alkyl, heteroalkyl, heteroalkenyl, heterocycloalkyl, and heteroaryl.

In one embodiment, one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is methyl.

In one embodiment, one of R₂ₐ and R_{2b} is H, and the other of R₂ₐ and R_{2b} is a C₁₋₁₀ alkyl, optionally substituted with one or more groups selected from the group consisting of halo, aryl, amino, alkyl, heteroalkyl, heteroalkenyl, heterocycloalkyl, and heteroaryl.

In another embodiment, one of R₂ₐ and R_{2b} is H, and the other of R₂ₐ and R_{2b} is methyl, ethyl, propyl, or isopropyl.

In one embodiment, one of R₂ₐ and R_{2b} is H and the other of R₂ₐ and R_{2b} is methyl.

In another aspect, the invention is directed to a compound of formula A-3, A-4, A-4A, A-4A 1, A-4A2, A-4A3, A-4B, A-4B1, A-4B2, A-4B3, B-2, B-3, B-4, B-5, B-6, B-7, B-8, and B-9.

In another aspect, the invention is directed to a compound as depicted in Table 1.

TABLE 1

| | |
|---|---|
| S1-5 | 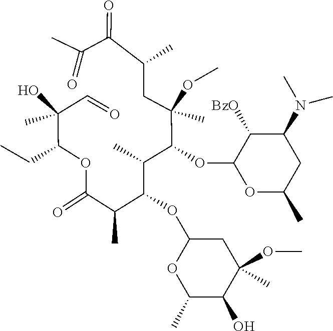 |
| S1-6 | 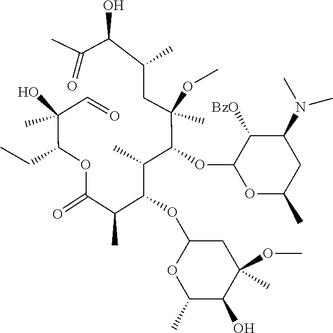 |
| S1-8 | 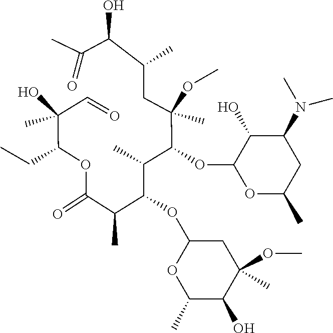 [SMILES: CC[C@@H](OC(=O)[C@H](C)[C@@H](OC1C[C@@](C)(OC)[C@@H](O)[C@H](C)O1)[C@H](C)[C@@H](OC1O[C@H](C)C[C@H](N(C)C)[C@H]1O)[C@@](C)(C[C@@H](C)[C@H](O)C(C)=O)OC)[C@@](C)(O)C=O] |
| S2-3 | 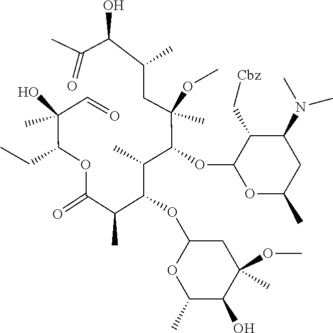 [SMILES: CC[C@@H](OC(=O)[C@H](C)[C@@H](OC1C[C@@](C)(OC)[C@@H](O)[C@H](C)O1)[C@H](C)[C@@H](OC1O[C@H](C)C[C@H](N(C)C)[C@H]1CC(=O)OCc1ccccc1)[C@@](C)(C[C@@H](C)[C@H](O)C(C)=O)OC)[C@@](C)(O)C=O] |
| S3-1a | 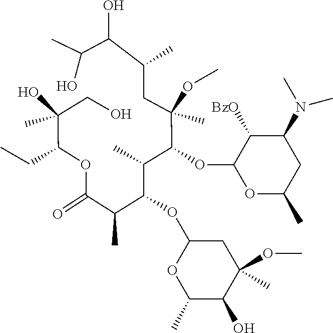 |
| S3-1b | 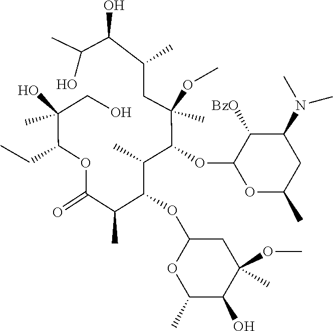 |
| S3-2 | 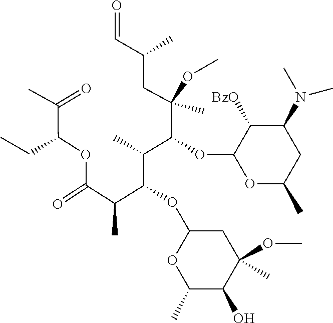 |
| S3-3 | 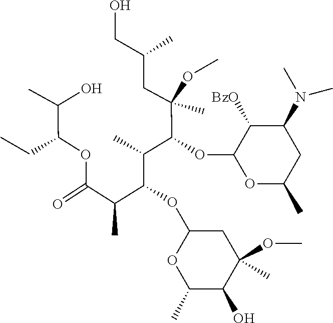 |
| S3-4 | 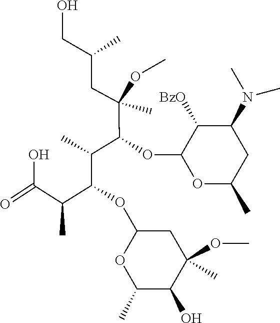 |
| S3-5 | 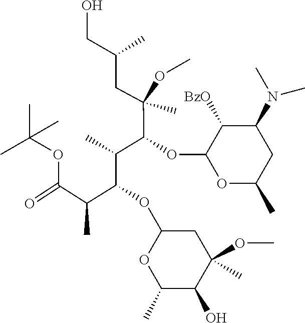 |
| S3-6 | 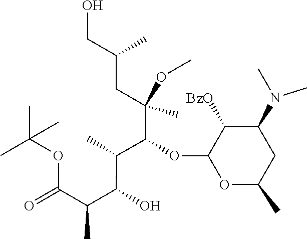 |
| S4-4 | 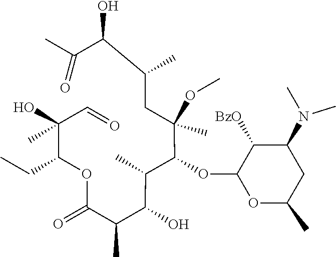 |
| S4-5 | 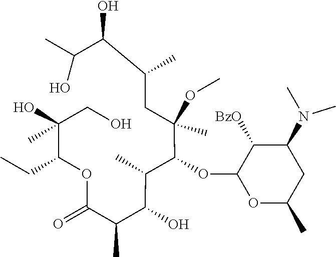 |
| S4-6 | 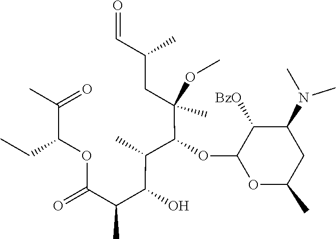 |
| S4-7 | 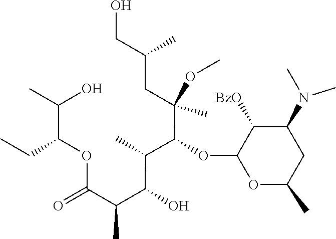 |
| S4-8 | 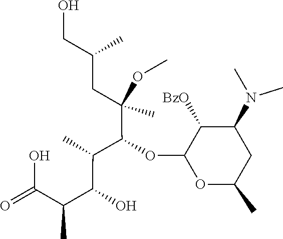 |
| S4-9 | 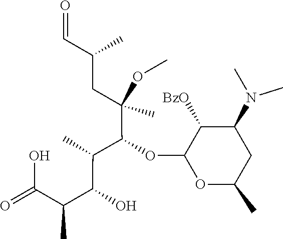 |
| S4-10 | 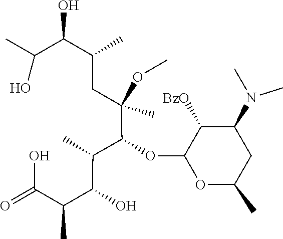 |
| S4-11 | 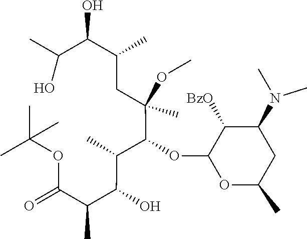 |
| S4-12 | 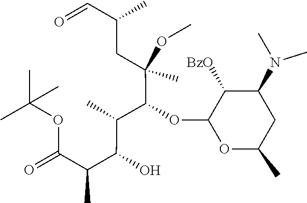 |
| S5-2 | 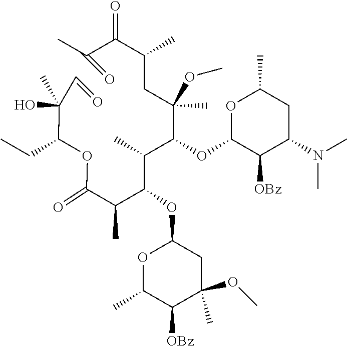 |
| S5-3 | 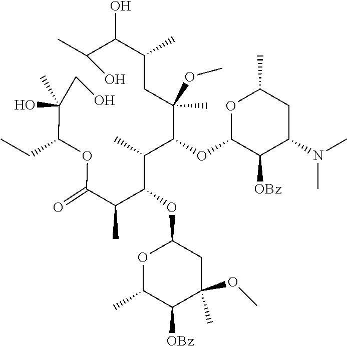 |
| S5-4 | 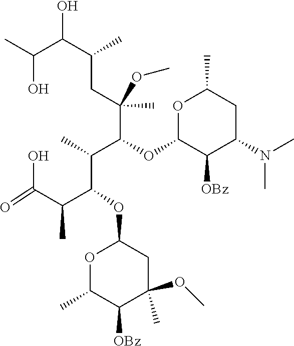 |
| S5-5 | 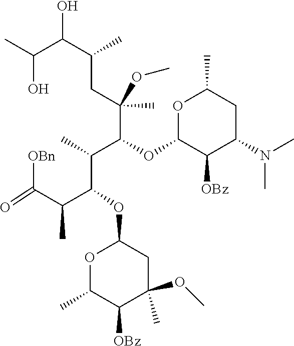 |
| S5-6 | 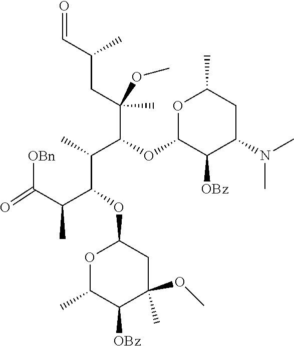 |
| S5-7 | 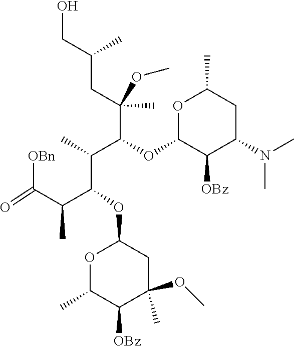 |
| S5-9 | 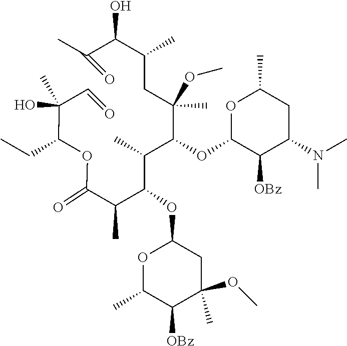 |
| S5-10 | 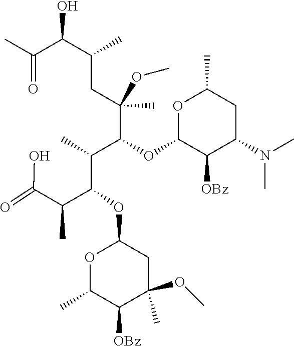 |
| S5-11 | 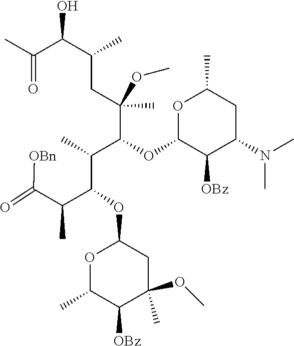 |
| S6-1 | 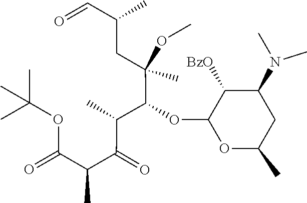 |

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Ozonolysis and Preparation of Intermediates

### Ozonolysis Route 1

Scheme 1 depicts the ozonolysis products S1-5, S1-6, and S1-8 commencing from S1-1, through intermediates that are optionally protected at the hydroxyl group of the C-5 desosamine. In the process, S1-1 was converted to S1-2 according to known methods (see, e.g., WO 2009/053259). In one alternative, the hydroxyl group of the C-5 desosamine of S1-2 was then protected, for example, the hydroxyl group of the C-5 desosamine was converted to a benozyl group according to known methods to provide S1-3. S1-3 was then subjected to ozonolyis in dichloromethane in the presence of an acid such as trifluoracetic acid. Quenching with dimethyl sulfide provided the ozonized product S1-5. Alternatively, the C-9 ketone in S1-3 was reduced to the corresponding alcohol S1-4, which after ozonolysis as described previously, provided S1-6. In another alternative, S1-2 was reduced to provide alcohol S1-7 and then S1-8 after subsequent ozonolysis.

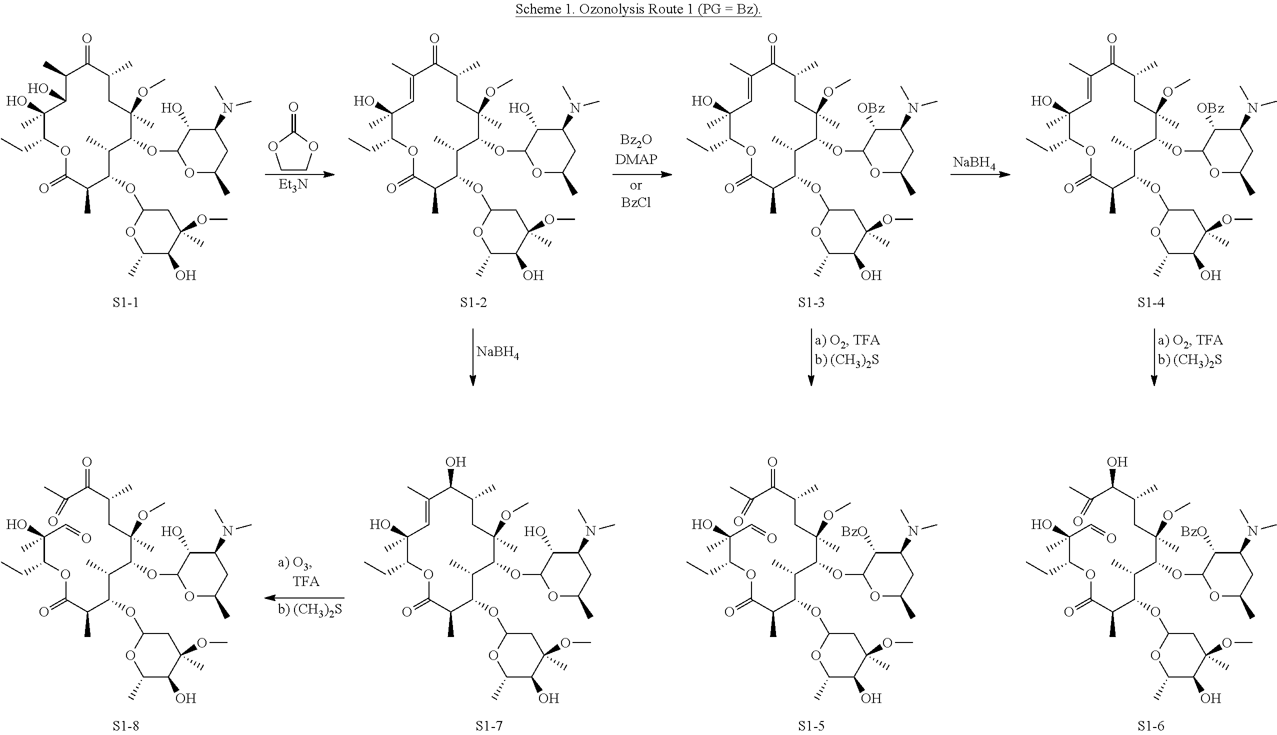

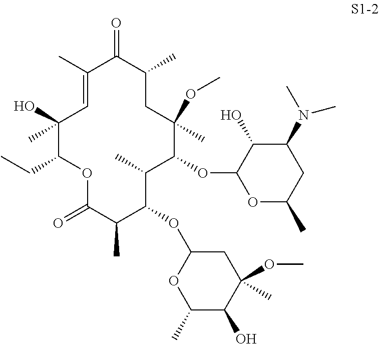 [SMILES: CC[C@H]1OC(=O)[C@H](C)[C@@H](OC2C[C@@](C)(OC)[C@@H](O)[C@H](C)O2)[C@H](C)[C@@H](OC2O[C@H](C)C[C@H](N(C)C)[C@H]2O)[C@](C)(OC)C[C@@H](C)C(=O)/C(C)=C/[C@]1(C)O]

(3R,4S,5S,6R,7R,9R,13S,14R,E)-6-(((3R,4S,6R)-4-(dimethylamino)-3-hydroxy-6-methyltetrahydro-2H-pyran-2-yl)oxy)-14-ethyl-13-hydroxy-4-(((4R,5S,6S)-5-hydroxy-4-methoxy-4,6-dimethyltetrahydro-2H-pyran-2-yl)oxy)-7-methoxy-3,5,7,9,11,13-hexamethyloxacyclotetradec-11-ene-2,10-dione (S1-2). S1-2 was synthesized by known methods (PCT Int. Appl., 2009053259, 30 Apr. 2009).

163.2 kg Clarithromycin and 180.4 kg TEA were charged into a glass-lined reactor (1000 L). The temperature was adjusted to 75-95° C. (white suspension). The reaction mixture was agitated at 45-50° C. for approximately 10 to 20 mins to provide a white suspension. 111.6 kg Ethylene carbonate (5.78 eq.) and 60.3 kg TEA were then charged into the glass-lined reactor (1000 L). The temperature was adjusted the temperature to 85-95° C. and the mixture was agitated for approximately 20-22 hrs. IPC: 9.2% residual Clarithromycin. 53.8 kg Ethylene carbonate was charged into the glass-lined reactor (1000 L). The temperature was adjusted to 85-95° C. and the mixture was agitated for 7-9 hrs. IPC, 1.8% residual Clarithromycin. The reaction mixture was then agitated at 85-95° C. for another 4 hrs. IPC, 0.91% residual Clarithromycin. The temperature was then adjusted to 45-55° C. 122.9 kg H₂O was then added dropwise to the reaction mixture at 45-55° C. The mixture was stirred at approximately 45 to 55° C. for 1 hour. The temperature was adjusted to 15-25° C. and agitated for 15-17 hrs. The reaction mixture was then to remove liquid. The filter cake was washed with 163.3 kg H₂O. The filter cake (light white solid) was sampled for IPC, S1-2: 96.0%. The cake was then dried at 45-55° C. for 23-25 hrs. 128.61 kg of S1-2 was obtained as a gray-white solid, 80.85% yield.

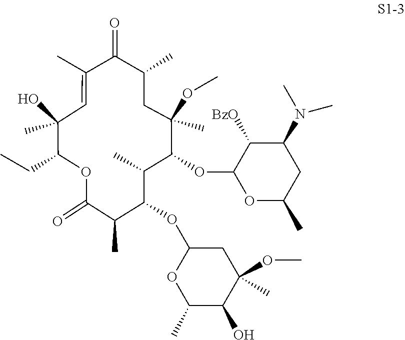

(3R,4S,6R)-4-(dimethylamino)-2-(((3R,4S,5S,6R,7R,9R,13S,14R,E)-14-ethyl-13-hydroxy-4-(((4R,5S,6S)-5-hydroxy-4-methoxy-4,6-dimethyltetrahydro-2H-pyran-2-yl)oxy)-7-methoxy-3,5,7,9,11,13-hexamethyl-2,10-dioxooxacyclotetradec-11-en-6-yl)oxy)-6-methyltetrahydro-2H-pyran-3-yl benzoate (S1-3). Preparation method 1: S1-2 (3.8 g, 5.2 mmol), benzoic anhydride (2.35 g, 10.4 mmol), and DMAP (63.5 mg, 0.52 mmol) were dissolved in dichloromethane (DCM, 30 mL) and triethylamine (2.16 mL, 15.6 mmol) was added. The mixture was refluxed overnight. The solution was washed with NaHCO₃ (saturated, aqueous, 2 times), dried over Na₂SO₄, filtered, and concentrated. The material was purified by ISCO (A: DCM, B: 20% MeOH in DCM with 0.5% NH₄OH, gradient elution from 0 to 40% B), yielding the title compound (3.9 g, 90%). MS (ESI+) m/z: 834.21 [M+H]⁺; ¹H NMR (400 MHz, Chloroform-d) δ 8.02-7.92 (m, 2H), 7.57-7.48 (m, 1H), 7.40 (t, 2H), 6.47 (d, 1H), 5.05 (dd, 1H), 4.94 (dd, 1H), 4.80 (d, 1H), 4.64 (d, 1H), 4.03 (dq, 1H), 3.93 (d, 1H), 3.64 (d, 1H), 3.60-3.51 (m, 1H), 3.40 (s, 3H), 3.14 (s, 3H), 3.03 (q, 1H), 2.86-2.74 (m, 1H), 2.68 (p, 1H), 2.44-2.30 (m, 1H), 2.26 (s, 7H), 1.98 (s, 3H), 1.92-1.83 (m, 2H), 1.81-1.72 (m, 3H), 1.59 (dd, 1H), 1.49-1.36 (m, 3H), 1.35-1.22 (m, 16H), 1.17 (dd, 6H), 0.85 (t, 3H), 0.73 (d, 3H).

Preparation method 2: To a solution of S1-2 (20 g, 27.3 mmol) in DCM (160 mL) was added triethylamine (5.68 mL, 40.9 mmol), and the mixture was cooled in an ice-water bath. Benzoyl chloride (4.1 mL, 34.1 mmol) was added dropwise. The solution was allowed to warm to rt and stir for 1 h. The mixture was then heated to reflux overnight. The solution was washed with NaHCO₃ (saturated, aqueous, 2 times), dried over Na₂SO₄, filtered and concentrated. The residue was purified by ISCO (120 g silica gel column, A: DCM, B: 20% MeOH in DCM with 0.5% NH₄OH, gradient elution from 0 to 40% B), yielding the title compound (22.7 g, 100%).

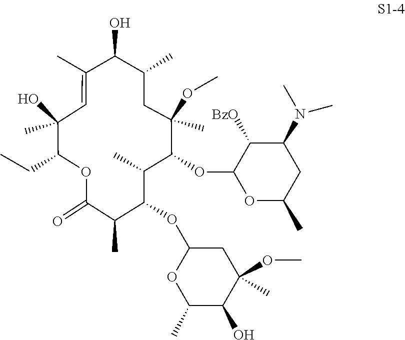

(3R,4S,6R)-4-(dimethylamino)-2-(((3R,4S,5S,6R,7R,9R,10S,13S,14R,E)-14-ethyl-10,13-dihydroxy-4-(((4R,5S,6S)-5-hydroxy-4-methoxy-4,6-dimethyltetrahydro-2H-pyran-2-yl)oxy)-7-methoxy-3,5,7,9,11,13-hexamethyl-2-oxooxacyclotetradec-1-en-6-yl)oxy)-6-methyltetrahydro-2H-pyran-3-yl benzoate (S1-4). NaBH₄ (270 mg, 5.44 mmol) was added to a solution of S1-3 (2.27 g, 2.72 mmol) in EtOH (20 mL). After 1 h, the mixture was concentrated by rotovap and diluted with EtOAc. The mixture was washed with water and brine, dried over Na₂SO₄, filtered, and concentrated. Purification by ISCO (A: DCM, B: 20% MeOH in DCM with 0.5% NH₄OH, gradient elution from 0 to 50% B), gave the title compound (1.86 g, 82%). MS (ESI+) m/z: 836.23 [M+H]⁺: ¹H NMR (400 MHz, Chloroform-d) δ 8.05-7.93 (m, 2H), 7.54 (t, 1H), 7.42 (t, 2H), 5.78 (s, 1H), 5.05 (dd, 1H), 4.79 (dd, 1H), 4.74 (d, 1H), 4.70 (d, 1H), 4.01 (dq, 1H), 3.94 (s, 1H), 3.79-3.70 (m, 2H), 3.56 (dtd, 1H), 3.44 (s, 3H), 3.17 (s, 3H), 3.02 (t, 1H), 2.88 (s, 1H), 2.79 (td, 1H), 2.67 (p, 1H), 2.40 (d, 1H), 2.27 (s, 6H), 2.18 (dd, 2H), 1.86 (d, 1H), 1.78-1.72 (m, 1H), 1.69-1.55 (m, 5H), 1.42 (dd, 2H), 1.34 (s, 3H), 1.29-1.24 (m, 12H), 1.14 (d, 3H), 1.10 (d, 3H), 0.81 (t, 3H), 0.73 (d, 3H).

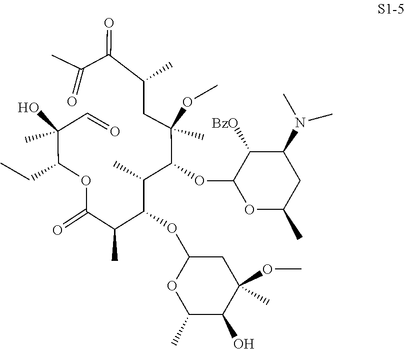

(3R,4S,6R)-4-(dimethylamino)-2-(((2R,3S,4S,5R,6R,8R)-1-(((2R,3R)-2-hydroxy-2-methyl-1-oxopentan-3-yl)oxy)-3-(((4R,5S,6S)-5-hydroxy-4-methoxy-4,6-dimethyltetrahydro-2H-pyran-2-yl)oxy)-6-methoxy-2,4,6,8-tetramethyl-1,9,10-trioxoundecan-5-yl)oxy)-6-methyltetrahydro-2H-pyran-3-yl benzoate (S1-5). Trifluoroacetic acid (2.28 g, 20 mmol) in DCM (8 mL) was added slowly into a solution of S1-3 (12.9 g, 15.4 mmol) in DCM (128 mL). The solution was cooled in a dry ice-acetone bath (approximately −60° C. internal temp.), and 03 was bubbled into the solution until a blue color was observed (approximately 10 min). Nitrogen was bubbled through the solution for 3 min until the blue color was no longer observed. Me₂S (3.39 mL) was added, and the solution was stirred in the dry ice-acetone bath for 20 min. The solution was washed with saturated, aqueous NaHCO₃. The organic layer was dried over Na₂SO₄, was filtered, and was concentrated to give crude S1-5. MS (ESI+) m/z: 866.29 [M+H]⁺.

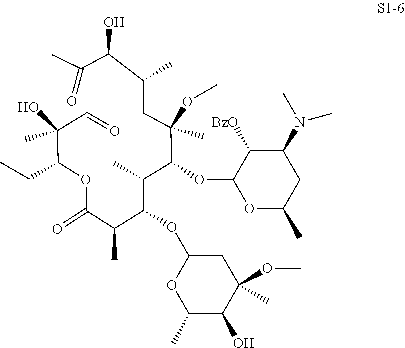

(3R,4S,6R)-4-(dimethylamino)-2-(((2R,3S,4S,5R,6R,8R,9S)-9-hydroxy-1-(((2R,3R)-2-hydroxy-2-methyl-1-oxopentan-3-yl)oxy)-3-(((4R,5S,6S)-5-hydroxy-4-methoxy-4,6-dimethyltetrahydro-2H-pyran-2-yl)oxy)-6-methoxy-2,4,6,8-tetramethyl-1,10-dioxoundecan-5-yl)oxy)-6-methyltetrahydro-2H-pyran-3-yl benzoate (S1-6). Trifluoroacetic acid (161 mg, 1.42 mmol) was added to a solution of S1-4 (960 mg, 1.14 mmol) in DCM (11.3 mL), and the solution was cooled in a dry ice-acetone bath. 03 was bubbled into the solution until a blue color was observed. Nitrogen was bubbled through the solution until the blue color was no longer observed. Me₂S (0.67 mL) was added, and the solution was stirred in the dry ice-acetone bath for 40 min. The solution was washed with saturated, aqueous NaHCO₃. The organic layer was dried over Na₂SO₄, filtered, and concentrated to give crude S1-6. MS (ESI+) m/z: 868.25 [M+H]⁺.

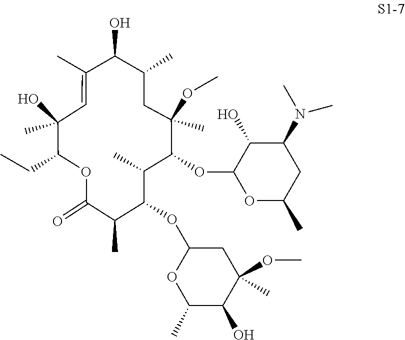 [SMILES: CC[C@H]1OC(=O)[C@H](C)[C@@H](OC2C[C@@](C)(OC)[C@@H](O)[C@H](C)O2)[C@H](C)[C@@H](OC2O[C@H](C)C[C@H](N(C)C)[C@H]2O)[C@](C)(OC)C[C@@H](C)[C@H](O)/C(C)=C/[C@]1(C)O]

(3R,4S,5S,6R,7R,9R,10S,13S,14R,E)-6-(((3R,4S,6R)-4-(dimethylamino)-3-hydroxy-6-methyltetrahydro-2H-pyran-2-yl)oxy)-14-ethyl-10,13-dihydroxy-4-(((4R,5S,6S)-5-hydroxy-4-methoxy-4,6-dimethyltetrahydro-2H-pyran-2-yl)oxy)-7-methoxy-3,5,7,9,11,13-hexamethyloxacyclotetradec-11-en-2-one (S1-7). S1-2 (8 g, 10.9 mmol) was dissolved in THF (50 mL) and EtOH (10 mL) and NaBH₄ (824 mg, 21.8 mmol) were added. The mixture was stirred overnight and was concentrated. The residue was triturated with DCM, the solid was removed by filtration, and the filtrate was concentrated and purified by ISCO (A: DCM, B: 20% MeOH in DCM with 0.5% NH₄OH, gradient elution from 0 to 100% B), yielding the title compound (6.8 g, 85%). MS (ESI+) m/z: 732.4 [M+H]⁺.

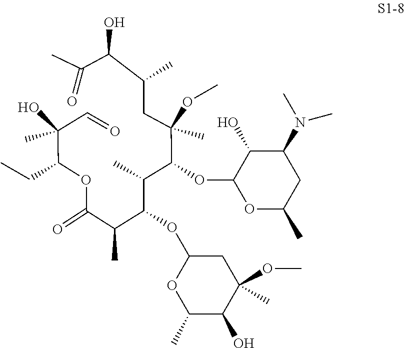 [SMILES: CC[C@@H](OC(=O)[C@H](C)[C@@H](OC1C[C@@](C)(OC)[C@@H](O)[C@H](C)O1)[C@H](C)[C@@H](OC1O[C@H](C)C[C@H](N(C)C)[C@H]1O)[C@@](C)(C[C@@H](C)[C@H](O)C(C)=O)OC)[C@@](C)(O)C=O]

(2R,3R)-2-hydroxy-2-methyl-1-oxopentan-3-yl (2R,3S,4S,5R,6R,8R,9S)-5-(((3R,4S,6R)-4-(dimethylamino)-3-hydroxy-6-methyltetrahydro-2H-pyran-2-yl)oxy)-9-hydroxy-3-(((4R,5S,6S)-5-hydroxy-4-methoxy-4,6-dimethyltetrahydro-2H-pyran-2-yl)oxy)-6-methoxy-2,4,6,8-tetramethyl-10-oxoundecanoate (S1-8). Trifluoroacetic acid (39 mg, 0.34 mmol) was added to a solution of S1-7 (200 mg, 0.27 mmol) in DCM (5 mL), and the solution was cooled in a dry ice-acetone bath. 03 was bubbled into the solution until a blue color was observed. Nitrogen was bubbled through the solution until the blue color was no longer observed. Me₂S (0.3 mL) was added, and the solution was stirred in the dry ice-acetone bath for 1 h. The solution was washed with saturated, aqueous NaHCO₃. The organic layer was dried over Na₂SO₄, was filtered, and was concentrated to give crude S1-8. MS (ESI+) m/z: 764.2 [M+H]⁺.

### Ozonolysis Route 2

Scheme 2 provides an ozonolysis route to S2-3 by first protecting compound S1-2 with a Cbz protecting group instead of the benzyl protecting group previously described. According to Scheme 2, S1-2 (cf. Scheme 1) is Cbz-protected at the hydroxyl group of the C-5 desosamine to provide S2-1. Reduction to S2-2 followed by ozonolysis as described previously provided S2-3.

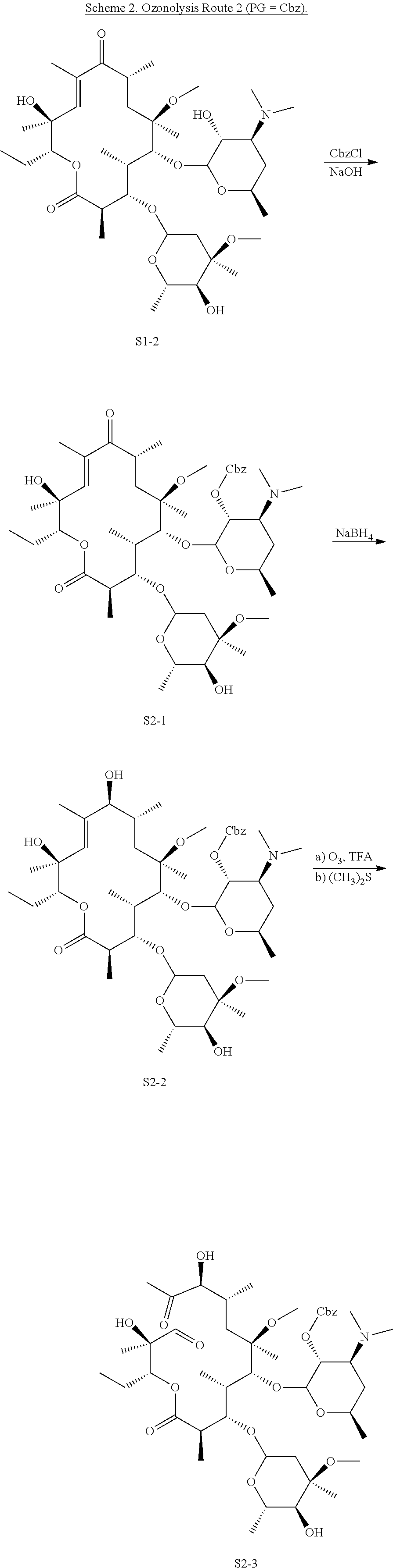

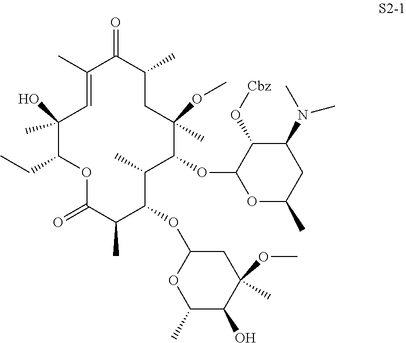 [SMILES: CC[C@H]1OC(=O)[C@H](C)[C@@H](OC2C[C@@](C)(OC)[C@@H](O)[C@H](C)O2)[C@H](C)[C@@H](OC2O[C@H](C)C[C@H](N(C)C)[C@H]2OC(=O)OCc2ccccc2)[C@](C)(OC)C[C@@H](C)C(=O)/C(C)=C/[C@]1(C)O]

Benzyl ((3R,4S,6R)-4-(dimethylamino)-2-(((3R,4S,5S,6R,7R,9R,13S,14R,E)-14-ethyl-13-hydroxy-4-(((4R,5S,6S)-5-hydroxy-4-methoxy-4,6-dimethyltetrahydro-2H-pyran-2-yl)oxy)-7-methoxy-3,5,7,9,11,13-hexamethyl-2,10-dioxooxacyclotetradec-11-en-6-yl)oxy)-6-methyltetrahydro-2H-pyran-3-yl) carbonate (S2-1): To a solution of S1-2 (500 mg, 0.684 mmol) in THF/H₂O (1:1, 6.8 mL) was added NaOH (20% aqueous solution, 409 mg, 2.05 mmol) followed by benzyl chloroformate (0.24 mL, 1.7 mmol). After 30 min, the reaction mixture was extracted with DCM, and the organic layer was washed with brine, dried over Na₂SO₄, filtered, and concentrated. Purification by ISCO (A: DCM, B: 20% MeOH in DCM with 0.5% NH₄OH, gradient elution from 0 to 40% B), yielded the title compound (516 mg, 87%). MS (ESI+) m/z: 864.29 [M+H]⁺.

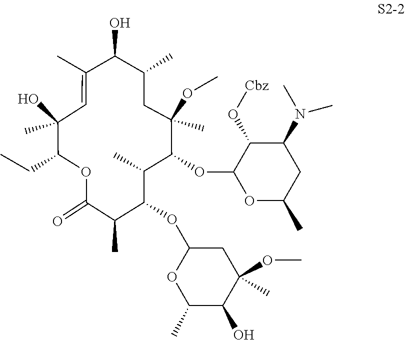 [SMILES: CC[C@H]1OC(=O)[C@H](C)[C@@H](OC2C[C@@](C)(OC)[C@@H](O)[C@H](C)O2)[C@H](C)[C@@H](OC2O[C@H](C)C[C@H](N(C)C)[C@H]2OC(=O)OCc2ccccc2)[C@](C)(OC)C[C@@H](C)[C@H](O)/C(C)=C/[C@]1(C)O]

Benzyl ((3R,4S,6R)-4-(dimethylamino)-2-(((3R,4S,5S,6R,7R,9R,10S,13S,14R,E)-14-ethyl-10,13-dihydroxy-4-(((4R,5S,6S)-5-hydroxy-4-methoxy-4,6-dimethyltetrahydro-2H-pyran-2-yl)oxy)-7-methoxy-3,5,7,9,11,13-hexamethyl-2-oxooxacyclotetradec-11-en-6-yl)oxy)-6-methyltetrahydro-2H-pyran-3-yl) carbonate (S2-2): To a solution of S2-1 (500 mg, 0.578 mmol) in EtOH (5.8 mL) was added NaBH₄ (43.5 mg, 1.15 mmol). After 1 h, the reaction mixture was diluted with EtOAc, washed with water and brine, was dried over Na₂SO₄, was filtered, and was concentrated. Purification by ISCO (A: DCM, B: 20% MeOH in DCM with 0.5% NH₄OH, gradient elution from 0 to 50% B), gave the title compound (460 mg, 92%). MS (ESI+) m/z: 866.26 [M+H]⁺.

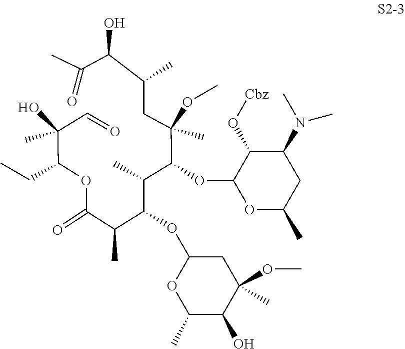 [SMILES: CC[C@@H](OC(=O)[C@H](C)[C@@H](OC1C[C@@](C)(OC)[C@@H](O)[C@H](C)O1)[C@H](C)[C@@H](OC1O[C@H](C)C[C@H](N(C)C)[C@H]1OC(=O)OCc1ccccc1)[C@@](C)(C[C@@H](C)[C@H](O)C(C)=O)OC)[C@@](C)(O)C=O]

(2R,3R)-2-hydroxy-2-methyl-1-oxopentan-3-yl (2R,3S,4S,5R,6R,8R,9S)-5-(((3R,4S,6R)-3-(((benzyloxy)carbonyl)oxy)-4-(dimethylamino)-6-methyltetrahydro-2H-pyran-2-yl)oxy)-9-hydroxy-3-(((4R,5S,6S)-5-hydroxy-4-methoxy-4,6-dimethyltetrahydro-2H-pyran-2-yl)oxy)-6-methoxy-2,4,6,8-tetramethyl-10-oxoundecanoate (S2-3). Trifluoroacetic acid (33 mg, 0.29 mmol) was added to a solution of S2-3 (200 mg, 0.23 mmol) in DCM (5 mL), and the solution was cooled in a dry ice-acetone bath. 03 was bubbled into the solution until a blue color was observed. Nitrogen was bubbled through the solution until the blue color was no longer observed. Me₂S (0.3 mL) was added, and the solution was stirred in the dry ice-acetone bath for 30 min. The solution was washed with saturated, aqueous NaHCO₃. The organic layer was dried over Na₂SO₄, was filtered, and was concentrated to give crude S2-3. MS (ESI+) m/z: 898.25 [M+H]⁺.

Conversion of S1-5 or S1-6 (Ozonolysis Route 1, Scheme 1) to t-Butyl Ester S3-6 Ozonolysis products S1-5 and S1-6 are readily converted to azalide building block S3-6 according to Scheme 3. According to Scheme 3, reduction of S1-5 or S1-6 followed by periodate oxidation of the resulting alcohol S3-1 provided diketone S3-2. Reduction of S3-2 provided S3-3. Ester hydrolysis of S3-3 using LiOH provided acid S3-4, which was converted to the t-butyl ester S3-5. Removal of the cladinose residue at C-4 of S3-5 provided S3-6.

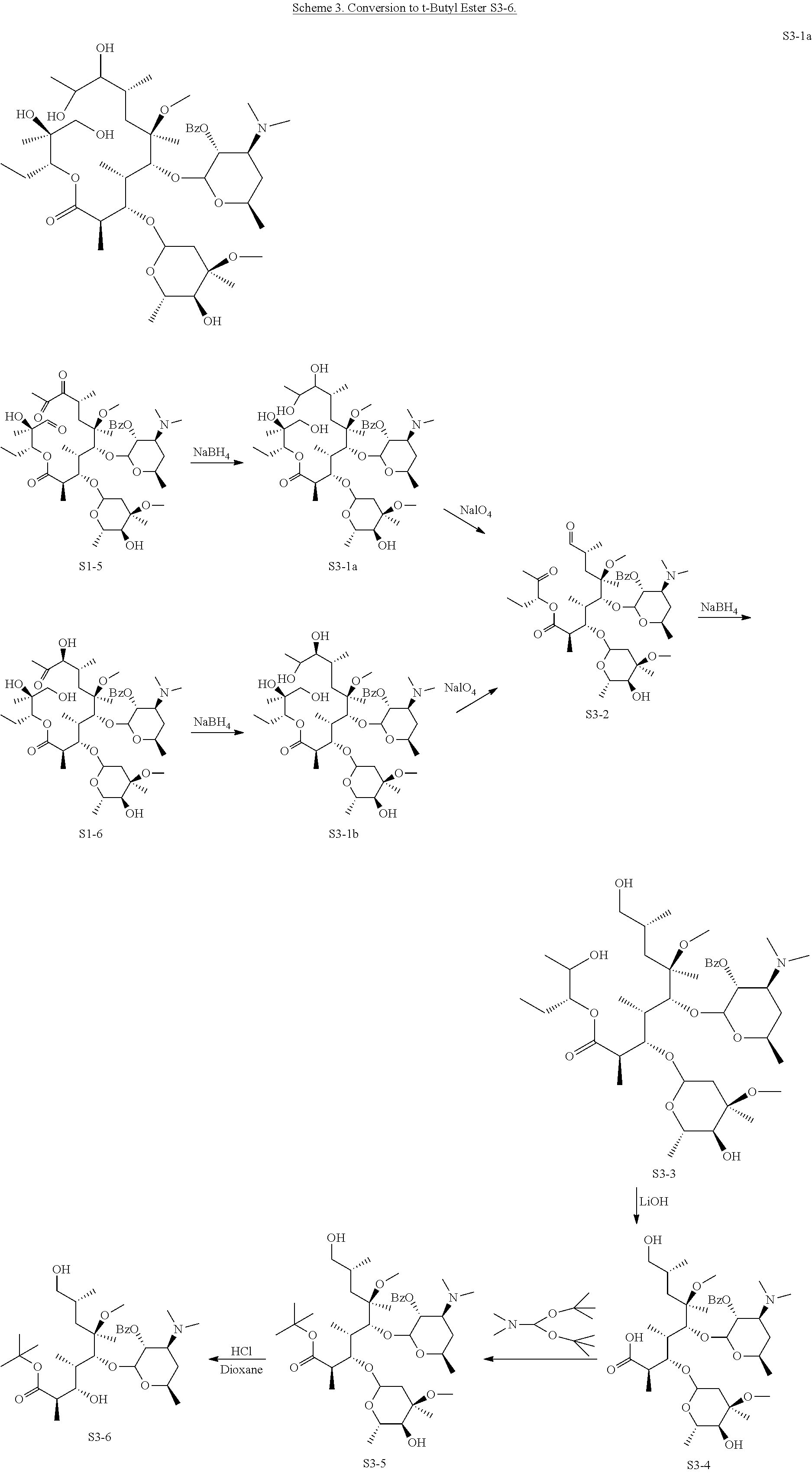

(3R,4S,6R)-2-(((2R,3S,4S,5R,6R,8R)-1-(((2S,3R)-1,2-dihydroxy-2-methylpentan-3-yl)oxy)-9,10-dihydroxy-3-(((4R,5S,6S)-5-hydroxy-4-methoxy-4,6-dimethyltetrahydro-2H-pyran-2-yl)oxy)-6-methoxy-2,4,6,8-tetramethyl-1-oxoundecan-5-yl)oxy)-4-(dimethylamino)-6-methyltetrahydro-2H-pyran-3-yl benzoate (S3-1a). A solution of S1-5 (12.9 g, 14.8 mmol) in EtOH (125 mL) was cooled to −25° C. for 10 min. Powdered NaBH₄ (1.14 g, 30.3 mmol) was added in portions. Upon complete addition, the reaction mixture was stirred between −10 to −20° C. for 1 h. NaHCO₃ (saturated, aqueous, 100 mL) was added to the solution, and the mixture was extracted with isopropyl acetate (500 mL). The organic layer was separated, washed with water (3×200 mL) and brine (100 mL), was dried over Na₂SO₄, filtered, and concentrated, yielding the title compound (12.9 g, 100% crude) as a white foam. MS (ESI+) m/z: 872.28 [M+H]⁺.

(3R,4S,6R)-2-(((2R,3S,4S,5R,6R,8R,9S)-1-(((2S,3R)-1,2-dihydroxy-2-methylpentan-3-yl)oxy)-9,10-dihydroxy-3-(((4R,5S,6S)-5-hydroxy-4-methoxy-4,6-dimethyltetrahydro-2H-pyran-2-yl)oxy)-6-methoxy-2,4,6,8-tetramethyl-1-oxoundecan-5-yl)oxy)-4-(dimethylamino)-6-methyltetrahydro-2H-pyran-3-yl benzoate (S3-1b) was synthesized using identical conditions as detailed above for compound S3-1a.

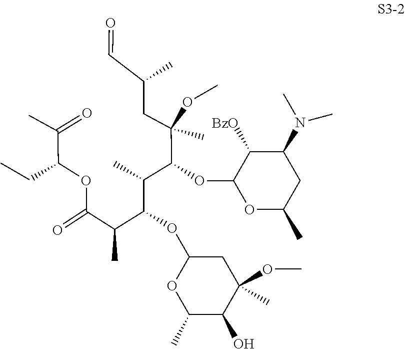

(3R,4S,6R)-4-(dimethylamino)-2-(((2R,3S,4S,5R,6R,8R)-3-(((4R,5S,6S)-5-hydroxy-4-methoxy-4,6-dimethyltetrahydro-2H-pyran-2-yl)oxy)-6-methoxy-2,4,6,8-tetramethyl-1,9-dioxo-1-(((R)-2-oxopentan-3-yl)oxy)nonan-5-yl)oxy)-6-methyltetrahydro-2H-pyran-3-yl benzoate (S3-2). A solution of crude S3-1a/b (5 g, 5.73 mmol) in EtOAc (57.3 mL) and water (4.2 mL) was cooled in an ice-water bath. NaIO₄ (4.4 g, 20.6 mmol) was added, and the mixture was stirred at it for 2 h. The solution was decanted, diluted with EtOAc, and washed with 10% Na₂S₂O₃, water, and brine. The organics were dried over Na₂SO₄, filtered, and concentrated. The material was purified by ISCO (A: DCM, B: 20% MeOH in DCM with 0.5% NH₄OH, gradient elution from 0 to 30% B), to yield the title compound (2.8 g, 62%) as a white foam. MS (ESI+) m/z: 794.15 [M+H]4; ¹H NMR (400 MHz, Chloroform-d) δ 9.27 (d, 1H), 8.11-7.93 (m, 2H), 7.58-7.49 (m, 1H), 7.42 (q, 2H), 5.07 (dd, 1H), 4.91-4.82 (m, 1H), 4.79 (d, 1H), 4.71 (d, 1H), 4.00 (ddt, 2H), 3.71 (d, 1H), 3.61 (dtt, 1H), 3.36 (s, 3H), 3.08-2.98 (m, 4H), 2.97-2.84 (m, 1H), 2.72 (qd, 1H), 2.44 (dpd, 1H), 2.30 (s, 6H), 2.17-2.07 (m, 4H), 1.85-1.69 (m, 4H), 1.58-1.39 (m, 3H), 1.33-1.20 (m, 14H), 1.12 (dd, 3H), 0.99 (d, 3H), 0.92 (t, 3H), 0.72 (d, 3H).

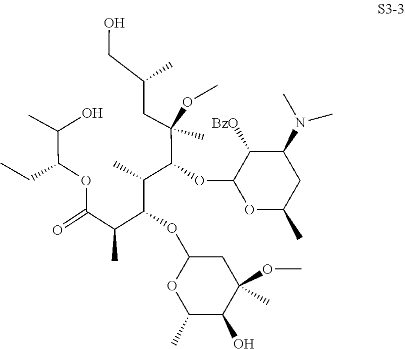

(3R,4S,6R)-4-(dimethylamino)-2-(((2R,3S,4S,5R,6R,8R)-9-hydroxy-3-(((4R,5S,6S)-5-hydroxy-4-methoxy-4,6-dimethyltetrahydro-2H-pyran-2-yl)oxy)-1-(((3R)-2-hydroxypentan-3-yl)oxy)-6-methoxy-2,4,6,8-tetramethyl-1-oxononan-5-yl)oxy)-6-methyltetrahydro-2H-pyran-3-yl benzoate (S3-3). To a solution of S3-2 (9.2 g, 11.5 mmol) in EtOH (100 mL) was added NaBH₄ (870 mg, 23 mmol), and the mixture was stirred at rt for 1 h. The solution was concentrated, and the residue was re-dissolved in EtOAc (200 mL), washed with water (2×100 mL) and brine (100 mL), dried over Na₂SO₄, filtered, and concentrated. The material was purified by ISCO (A: DCM, B: 20% MeOH in DCM with 0.5% NH₄OH, gradient elution from 0 to 80% B), yielding the title compound (7.2 g, 78%) as a white foam. MS (ESI+) m/z: 798.25 [M+H]⁺; ¹H NMR (400 MHz, Chloroform-d) δ 8.11-7.91 (m, 2H), 7.54 (t, 1H), 7.42 (t, 2H), 5.07 (dt, 1H), 4.82 (ddt, 1H), 4.70 (dtd, 2H), 4.35-4.07 (m, 1H), 4.01 (dtd, 1H), 3.76-3.21 (m, 12H), 2.86 (dtd, 1H), 2.70-2.51 (m, 1H), 2.39-2.09 (m, 8H), 1.95-1.72 (m, 2H), 1.69-1.04 (m, 27H), 1.02-0.80 (m, 6H), 0.78-0.54 (m, 3H).

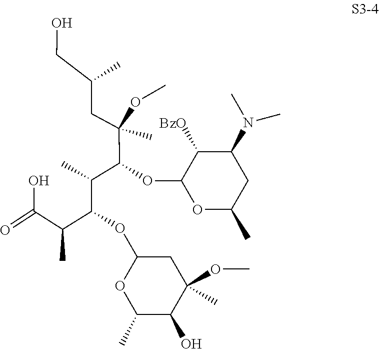

(2R,3S,4S,5R,6R,8R)-5-(((3R,4S,6R)-3-(benzoyloxy)-4-(dimethylamino)-6-methyltetrahydro-2H-pyran-2-yl)oxy)-9-hydroxy-3-(((4R,5S,6S)-5-hydroxy-4-methoxy-4,6-dimethyltetrahydro-2H-pyran-2-yl)oxy)-6-methoxy-2,4,6,8-tetramethylnonanoic acid (S3-4). A solution of S3-3 (900 mg, 1.12 mmol) in H₂O/THF (1:3, 19.3 mL) was stirred with LiOH (134 mg, 5.6 mmol) for 60 h at rt. Formic acid (257 mg, 5.6 mmol) was added, and the mixture was extracted with EtOAc (2×30 mL). The combined extracts were washed with water (2×30 mL) and brine (30 mL), dried over Na₂SO₄, filtered, and concentrated. The material was purified by ISCO (A: DCM, B. 20% MeOH in DCM, gradient elution from 0 to 100% B), to provide the title compound (570 mg, 71.5%). S3-3 (240 mg, 26.7%) was also recovered. MS (ESI+) m/z: 794.15 [M+H]⁺; ¹H NMR (400 MHz, Chloroform-d) δ 7.99 (d, 2H), 7.53 (t, 1H), 7.40 (t, 2H), 5.11 (dd, 1H), 4.69 (d, 4H), 3.98 (dt, 1H), 3.92 (t, 1H), 3.75 (d, 1H), 3.62 (dp, 1H), 3.53 (dd, 1H), 3.34 (s, 3H), 3.21 (s, 4H), 3.01 (d, 2H), 2.53-2.40 (m, 1H), 2.32 (s, 7H), 2.08 (td, 1H), 1.95-1.71 (m, 2H), 1.58-1.37 (m, 4H), 1.36-1.12 (m, 13H), 0.99 (d, 3H), 0.86 (d, 3H), 0.68 (d, 3H).

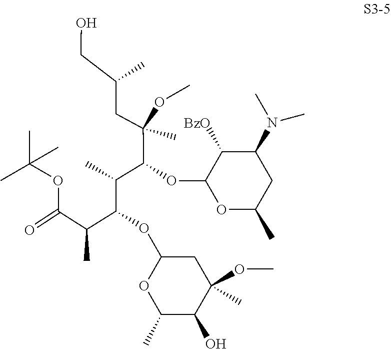

(3R,4S,6R)-2-(((2R,3S,4S,5R,6R,8R)-1-(tert-butoxy)-9-hydroxy-3-(((4R,5S,6S)-5-hydroxy-4-methoxy-4,6-dimethyltetrahydro-2H-pyran-2-yl)oxy)-6-methoxy-2,4,6,8-tetramethyl-1-oxononan-5-yl)oxy)-4-(dimethylamino)-6-methyltetrahydro-2H-pyran-3-yl benzoate (S3-5). A solution of S3-4 (160 mg, 0.224 mmol) in toluene (2.2 mL) was heated with N,N-dimethylformamide di-tert-butyl acetal (225 mg, 1.11 mmol) at 80° C. for 2 h. The reaction solution was used directly in next step. S3-5 was observed as the major product by LCMS. MS (ESI+) m/z: 768.26 [M+H]⁺.

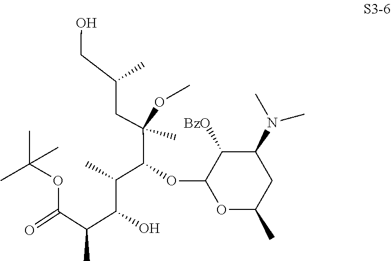

(3R,4S,6R)-2-(((2R,3S,4S,5R,6R,8R)-1-(tert-butoxy)-3,9-dihydroxy-6-methoxy-2,4,6,8-tetramethyl-1-oxononan-5-yl)oxy)-4-(dimethylamino)-6-methyltetrahydro-2H-pyran-3-yl benzoate (S3-6). The above reaction solution was stirred with HCl (4 M in dioxane, 0.36 mL, 1.45 mmol) for 15 min at it. The mixture was slowly quenched with saturated, aqueous NaHCO₃ and was extracted with EtOAc (2×10 mL). The combined extracts were washed with water (10 mL) and brine (10 mL), dried over Na₂SO₄, filtered, and concentrated. The material was purified by HPLC (Atlantis T3 column; Mobile phases: A: Water/0.1% formic acid, B: CH₃CN/0.1% formic acid, gradient elution with 2 to 40 to 90% B over 15 min) to give the title compound (83 mg, 61%) as a white foam. Formate salt. MS (ESI+) m/z: 610.2 [M+H]+; ¹H NMR (400 MHz, Chloroform-d) δ 8.06-8.00 (m, 2H), 7.57 (t, 1H), 7.43 (t, 2H), 5.20 (dd, 1H), 4.97 (d, 1H), 3.87 (d, 1H), 3.73-3.53 (m, 2H), 3.52-3.42 (m, 2H), 3.18 (s, 4H), 2.57 (s, 6H), 2.47-2.33 (m, 1H), 2.23-2.02 (m, 2H), 1.83 (qd, 1H), 1.58-1.46 (m, 1H), 1.46-1.40 (m, 1H), 1.38 (s, 9H), 1.34-1.21 (m, 8H), 1.18 (d, 3H), 1.14 (dd, 1H), 0.81 (d, 3H), 0.56 (d, 3H).

### Alternative Process for Preparing S3-6 (Scheme 3) via Ozonolysis Route 3

An alternative process for preparing the azalide building block S3-6 is depicted in Scheme 4. According to Scheme 4, the C-4 cladinose residue was first removed under acidic conditions to provide S4-1. Protection of the C-5 desosamine hydroxyl group, followed by reduction of the resulting compound S4-2, provided ozonolysis substrate S4-3. S4-5 was prepared via ozonolysis and reduction of S4-3 with and without isolation of the intermediate S4-4. Conversion of S4-5 to S3-6 was accomplished via several 4-step routes, including (i) hydrolysis, esterification, periodate oxidation, and reduction (S4-5→S4-10→S4-11→S4-12→S3-6); (ii) periodate oxidation, hydrolysis, esterification, reduction (S4-5→S4-6→S4-9→S4-10→S3-6); and (iii) periodate oxidation, reduction, hydrolysis, esterification (S4-5→S4-6→S4-7→S4-8→S3-6)

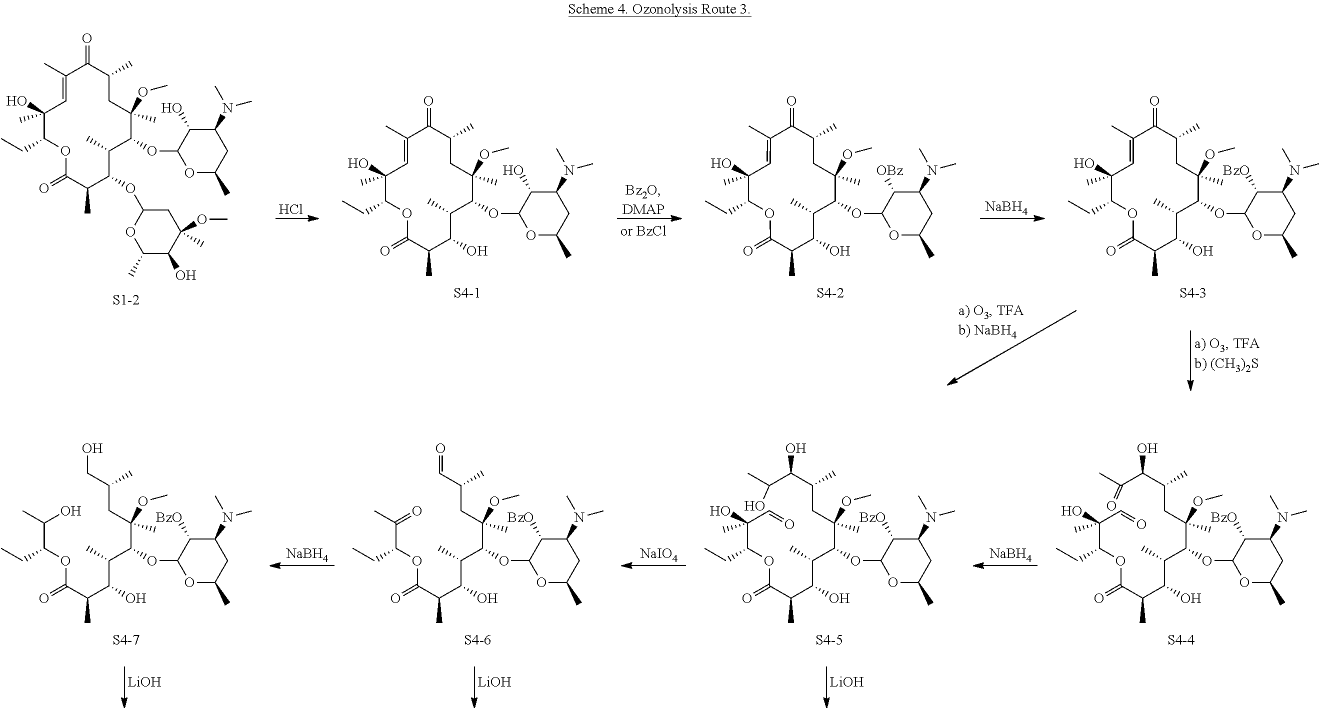

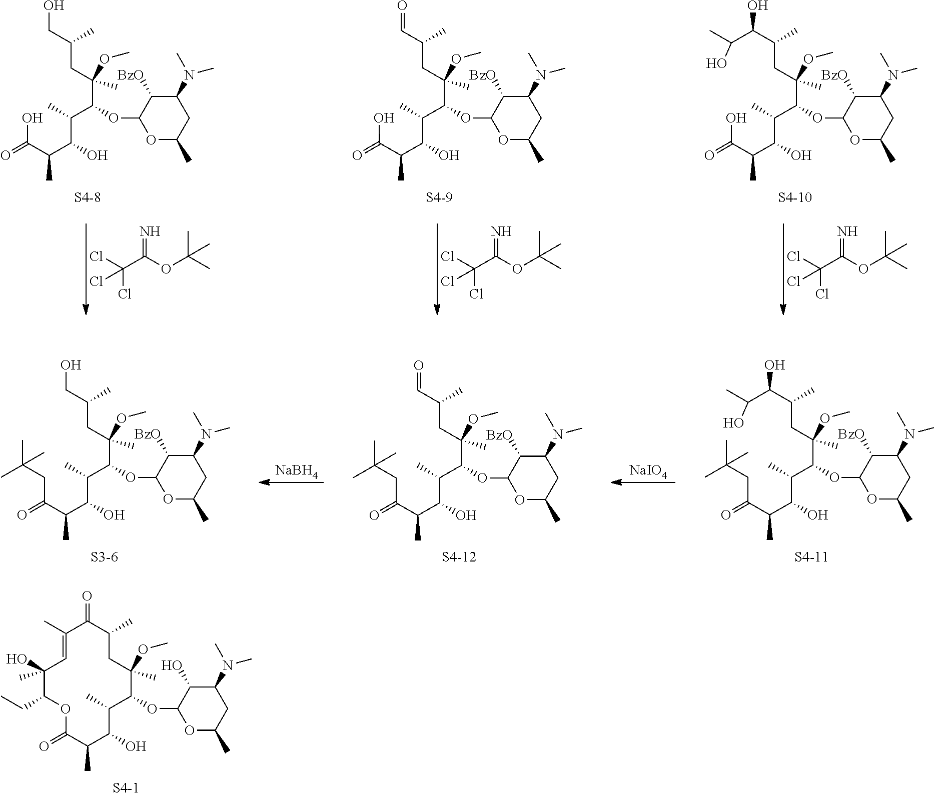

(3R,4S,5S,6R,7R,9R,13S,14R,E)-6-(((3R,4S,6R)-4-(dimethylamino)-3-hydroxy-6-methyltetrahydro-2H-pyran-2-yl)oxy)-14-ethyl-4,13-dihydroxy-7-methoxy-3,5,7,9,11,13-hexamethyloxacyclotetradec-11-ene-2,10-dione (S4-1). To a 2 L three-neck round-bottom flask equipped with an overhead stirrer, a temperature probe, and a nitrogen inlet and outlet was added S1-2 (97 g, 133 mmol) followed by methanol (500 mL). The reaction mixture was stirred until a clear solution formed. The solution was cooled with a water bath, and 6 N HCl (161 mL, 798 mmol, 6 eq.) was added over about 20 min while keeping the internal temperature between 20-25° C. After 2 h, UPLC-MS analysis showed >99% conversion. 28-30% Aqueous NH₄OH (200 mL) was added to adjust the pH to 9-10, followed by water (100 mL). The reaction mixture was stirred for an additional 16 h. The resulting white solid was collected by filtration, washed with MeOH/H₂O (2:3, 300 mL), and dried in a vacuum oven at 35-40° C. for 72 h. This gave the title compound (45 g, 59%) as a white solid. MS (ESI+) m/z: 572.27 [M+H]⁺.

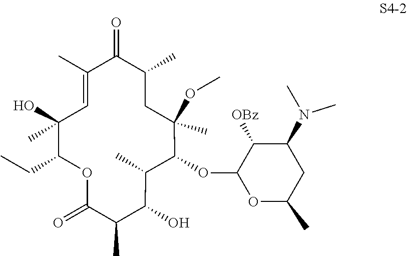

(3R,4S,6R)-4-(dimethylamino)-2-(((3R,4S,5S,6R,7R,9R,13S,14R,E)-14-ethyl-4,13-dihydroxy-7-methoxy-3,5,7,9,11,13-hexamethyl-2,10-dioxooxacyclotetradec-11-en-6-yl)oxy)-6-methyltetrahydro-2H-pyran-3-yl benzoate (S4-2). A 500 mL three-neck round-bottom flask equipped with a temperature probe, stir bar, and nitrogen inlet and outlet was charged with S4-1 (26 g, 45.4 mmol) followed by DCM (250 mL). The mixture was stirred at room temperature until a clear, light brown solution was obtained. Triethylamine (18.8 mL, 136 mmol, 3 eq) and benzoic anhydride (20.5 g, 90.8 mmol, 2 eq) were charged. The reaction mixture was heated at 40° C. for 24 h, at which point UPLC-MS showed >99% conversion. After cooling to room temperature, the reaction mixture was washed with 2 N NaOH (50 mL×2) and brine (50 mL), dried over Na₂SO₄, filtered, and concentrated. The residue was purified by silica gel column chromatography (0 to 100% of 80% DCM/20% MeOH/0.5% aqueous NH₄OH solution in DCM) to give the desired product (26 g, 85%) as a white foam. MS (ESI+) m/z: 676.27 [M+H].

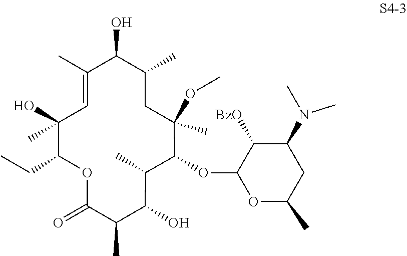

(3R,4S,6R)-4-(dimethylamino)-2-(((3R,4S,5S,6R,7R,9R,10S,13S,14R,E)-14-ethyl-4,10,13-trihydroxy-7-methoxy-3,5,7,9,11,13-hexamethyl-2-oxooxacyclotetradec-11-en-6-yl)oxy)-6-methyltetrahydro-2H-pyran-3-yl benzoate (S4-3). A 1 L round bottom flask equipped with a temperature probe, stir bar, and nitrogen inlet and outlet was charged with S4-2 (22 g, 32.5 mmol) followed by anhydrous EtOH (200 mL). The mixture was stirred at room temperature until a clear solution was obtained. NaBH₄ (2.45 g, 65 mmol, 2 eq) was added in one portion. The mixture was stirred at rt for 16 h, at which point UPLC-MS showed >99% conversion. Water (50 mL) and saturated, aqueous NH₄Cl (20 mL) were added to quench the reaction. The mixture was concentrated to remove most of the EtOH, was diluted with EtOAc (250 mL), and the layers were separated. The organic phase was dried over Na₂SO₄ and was concentrated. The residue was dissolved in MTBE (100 mL) at 40-50° C., and hexane (200 mL) was added at the same temperature. The mixture was cooled to room temperature and stirred overnight. The resulting white solid was collected by filtration and was dried at 35-40° C. in a vacuum oven for 72 h, yielding the title compound (12.7 g, 58%). MS (ESI+) m/z: 678.22 [M+H].

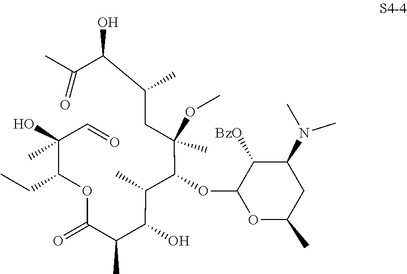

(3R,4S,6R)-2-(((2R,3S,4S,5R,6R,8R,9S)-3,9-dihydroxy-1-(((2R,3R)-2-hydroxy-2-methyl-1-oxopentan-3-yl)oxy)-6-methoxy-2,4,6,8-tetramethyl-1,10-dioxoundecan-5-yl)oxy)-4-(dimethylamino)-6-methyltetrahydro-2H-pyran-3-yl benzoate (S4-4). S4-3 (410 mg, 0.6 mmol) was added to a 100 mL round-bottom flask, followed by DCM (10 mL) and TFA (103 mg, 1.5 eq). The solution was cooled to −65° C. to −75° C. using a dry-ice/acetone cooling bath, and 03 was bubbled through the solution until the color changed to blue (about 5 min). Nitrogen was then bubbled through the solution until the blue color disappeared. Dimethyl sulfide (0.3 mL, 6.7 eq) was added, and the reaction mixture was stirred for 1 h. Saturated, aqueous NaHCO₃ (5 mL) was added and the two phases were separated. The DCM layer was dried over Na₂SO₄ and was concentrated, giving a white foam (490 mg), which was used in next step without further purification. MS (ESI+) m/z: 710.15 [M+H].

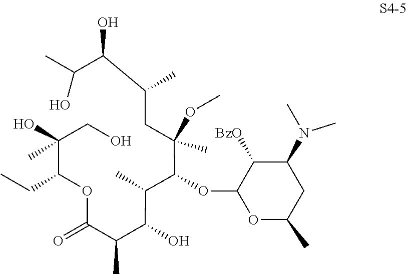

(3R,4S,6R)-2-(((2R,3S,4S,5R,6R,8R,9S)-1-(((2S,3R)-1,2-dihydroxy-2-methylpentan-3-yl)oxy)-3,9,10-trihydroxy-6-methoxy-2,4,6,8-tetramethyl-1-oxoundecan-5-yl)oxy)-4-(dimethylamino)-6-methyltetrahydro-2H-pyran-3-yl benzoate (S4-5).

Method 1: Crude S4-4 (490 mg, 0.6 mmol) was added to a 100 mL round-bottom flask, followed by EtOH (5 mL). NaBH₄ (51 mg, 2 eq) was added at room temperature, and the reaction mixture was stirred overnight. Saturated, aqueous NH₄Cl (2 mL) was added, and the mixture was stirred for 1 h. Water (2 mL) was added, and the mixture was stirred for 1 h. The mixture was concentrated to remove most of the EtOH and was extracted with EtOAc (15 mL×2). The combined extracts were dried and concentrated, giving a white foam (470 mg), which is used in next step without further purification. MS (ESI+) m/z: 714.10 [M+H].

Method 2: S4-3 (1.6 g, 2.4 mmol) was added to a 100 mL round-bottom flask, followed by MeOH (8 mL), DCM (10 mL) and TFA (0.24 mL, 1.5 eq). The solution was cooled to −78° C. to −45° C. 03 was bubbled through the solution for 20 min at which point UPLC-MS showed the starting material was consumed. NaBH₄ (446 mg, 5 eq) was added, and the reaction mixture was stirred at room temperature for 5 h. Saturated, aqueous NH₄Cl (2 mL), water (10 mL), and EtOAc (20 mL) were added, and the mixture was stirred for 16 h. The organic phase was separated, concentrated, and the residue was purified by silica gel column chromatography (0-100% gradient of 80% DCM/20% MeOH/0.5% aqueous NH₄OH in DCM), giving the desired product (400 mg, 24%) as a white foam. MS (ESI+) m/z: 714.25 [M+H].

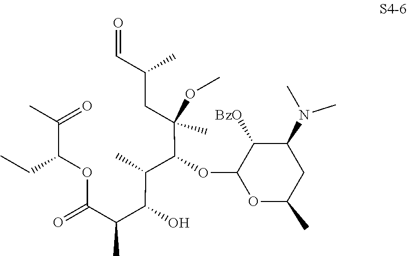

(3R,4S,6R)-4-(dimethylamino)-2-(((2R,3S,4S,5R,6R,8R)-3-hydroxy-6-methoxy-2,4,6,8-tetramethyl-1,9-dioxo-1-(((R)-2-oxopentan-3-yl)oxy)nonan-5-yl)oxy)-6-methyltetrahydro-2H-pyran-3-yl benzoate (S4-6). Crude S4-5 (470 mg, 0.6 mmol) was added to a 100 mL round-bottom flask, followed by EtOAc (5 mL) and water (6 drops). NaIO₄ (412 mg, 3 eq) was added, and the reaction mixture was stirred for 3 h. The mixture was diluted to 30 mL with EtOAc, washed with 13% NaCl solution (5 mL), dried, and concentrated to a thick oil (410 mg). The material was used in the next step without further purification. MS (ESI+) m/z: 636.19 [M+H].

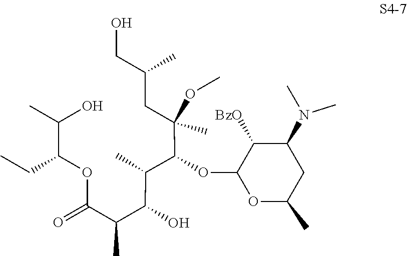

(3R,4S,6R)-2-(((2R,3S,4S,5R,6R,8R)-3,9-dihydroxy-1-(((3R)-2-hydroxypentan-3-yl)oxy)-6-methoxy-2,4,6,8-tetramethyl-1-oxononan-5-yl)oxy)-4-(dimethylamino)-6-methyltetrahydro-2H-pyran-3-yl benzoate (S4-7). Crude S4-6 (410 mg, 0.6 mmol) was added to a 100 mL round-bottom flask, followed by EtOH (5 mL). NaBH₄ (48 mg, 2 eq) was added, and the reaction mixture was stirred for 30 min. Water (2 mL) was added, and the mixture was stirred for 30 min. Ethyl acetate (20 mL) was added, and the two phases were separated. The organic phase was concentrated, and the residue was purified by silica gel column chromatography (0-100% gradient of 80% DCM/20% MeOH/0.5% aqueous NH₄OH in DCM), giving the desired product (180 mg, 44% from S3-3) as a white foam. MS (ESI+) m/z: 640.24 [M+H].

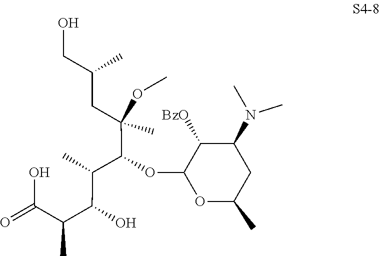

(2R,3S,4S,5R,6R,8R)-5-(((3R,4S,6R)-3-(benzoyloxy)-4-(dimethylamino)-6-methyltetrahydro-2H-pyran-2-yl)oxy)-3,9-dihydroxy-6-methoxy-2,4,6,8-tetramethylnonanoic acid (S4-8). S4-7 (150 mg, 0.23 mmol) was added to a 50 mL round-bottom flask, followed by THF (3 mL) and water (3 mL). LiOH (34 mg, 6 eq) was added, and the reaction mixture was stirred overnight. The reaction mixture was neutralized to pH approximately 5, was extracted with EtOAc (3×5 mL), and was concentrated to give the title compound (140 mg) as a white foam. MS (ESI+) m/z: 554.08 [M+H].

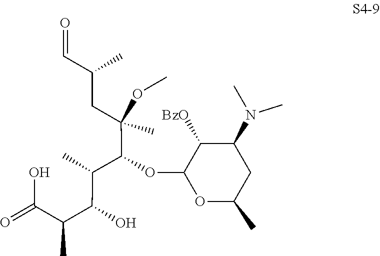

(2R,3S,4S,5R,6R,8R)-5-(((3R,4S,6R)-3-(benzoyloxy)-4-(dimethylamino)-6-methyltetrahydro-2H-pyran-2-yl)oxy)-3-hydroxy-6-methoxy-2,4,6,8-tetramethyl-9-oxononanoic acid (S4-9). S4-6 (170 mg, 0.27 mmol) was added to a 50 mL round-bottom flask, followed by MeCN (3 mL) and water (1 mL). LiOH (19 mg, 3 eq) was added, and the reaction mixture was stirred for 1 h. The reaction mixture was neutralized to pH approximately 5 and was saturated with solid NaCl. The mixture was extracted with EtOAc (3×5 mL), and the combined extracts were concentrated. The residue was purified by silica gel column chromatography (0-100% gradient of 80% DCM/20% MeOH/0.5% aqueous NH₄OH in DCM), giving the title compound (160 mg, 100%) as a white foam. MS (ESI+) m/z: 552.13 [M+H].

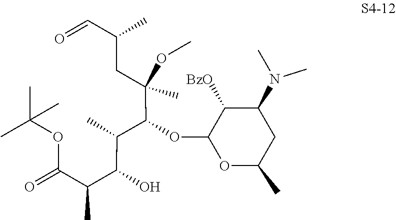

(2R,3S,4S,5R,6R,8R)-5-(((3R,4S,6R)-3-(benzoyloxy)-4-(dimethylamino)-6-methyltetrahydro-2H-pyran-2-yl)oxy)-3-hydroxy-6-methoxy-2,4,6,8-tetramethyl-9-oxononanoic acid (S4-12). S4-9 (30 mg, 0.27 mmol) was added to a 50 mL round-bottom flask, followed by THF/Hexane (0.3 mL/0.3 mL). Under nitrogen, tert-butyl 2,2,2-trichloroethanimidate (24 mg, 2 eq) and BF₃·Et₂O complex (1 drop) were added. After 24 h, additional tert-butyl 2,2,2-trichloroethanimidate (2 eq) and anhydrous DMF (0.1 mL) were added, and the reaction mixture was stirred for 16 h. The mixture was purified by silica gel column chromatography (0-100% gradient of 80% DCM/20% MeOH/0.5% aqueous NH₄OH in DCM), giving the title compound (14 mg, 42%) as a white foam. MS (ESI+) m/z: 608.10 [M+H].

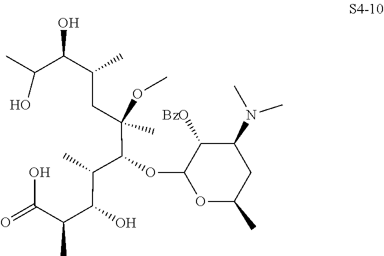

(2R,3S,4S,5R,6R,8R,9S)-5-(((3R,4S,6R)-3-(benzoyloxy)-4-(dimethylamino)-6-methyltetrahydro-2H-pyran-2-yl)oxy)-3,9,10-trihydroxy-6-methoxy-2,4,6,8-tetramethylundecanoic acid (S4-10). S4-5 (1.9 g, 2.7 mmol) was added to a 100 mL round-bottom flask, followed by CH₃CN (25 mL) and water (10 mL). LiOH (96 mg, 1.5 eq) was added, and the reaction mixture was stirred for 3 h at rt. The reaction mixture was concentrated to remove most of the ACN, and 6 N HCl was added dropwise until the solution reached pH approximately 6. EtOAc (30 mL) was added, followed by solid NaCl until the aqueous phase was saturated. The organic phase was separated, and the aqueous phase was extracted with EtOAc (10 mL). The combined organic phases were dried over Na₂SO₄, filtered, and concentrated to give the title compound (1.35 g, 85%) as a white foam, which was used in the next step without further purification. MS (ESI+) m/z: 598.16 [M+H].

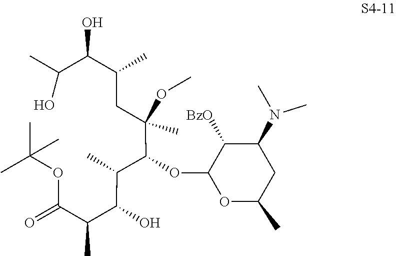

(3R,4S,6R)-2-(((2R,3S,4S,5R,6R,8R,9S)-1-(tert-butoxy)-3,9,10-trihydroxy-6-methoxy-2,4,6,8-tetramethyl-1-oxoundecan-5-yl)oxy)-4-(dimethylamino)-6-methyltetrahydro-2H-pyran-3-yl benzoate (S4-11). S4-10 (1.35 g, 2.25 mmol) was added to a 100 mL round-bottom flask, followed by anhydrous DCM (15 mL). Under nitrogen, tert-butyl 2,2,2-trichloroethanimidate (2 g, 4 eq) and BF₃·Et₂O complex (160 mg, 0.5 eq) were added, and the reaction mixture was stirred at room temperature for 48 h. After concentration, the residue was purified by silica gel column chromatography (0-100% gradient of 80% DCM/20% MeOH/0.5% aqueous NH₄OH in DCM), giving the title compound (1 g, 68%) as a white foam. MS (ESI+) m/z: 654.28 [M+H].

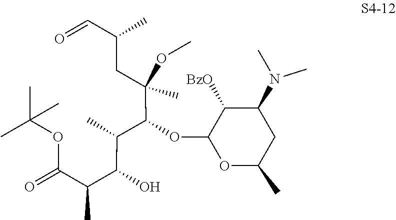

(2R,3S,4S,5R,6R,8R)-5-(((3R,4S,6R)-3-(benzoyloxy)-4-(dimethylamino)-6-methyltetrahydro-2H-pyran-2-yl)oxy)-3-hydroxy-6-methoxy-2,4,6,8-tetramethyl-9-oxononanoic acid (S4-12). S4-11 (34 mg, 0.05 mmol) was added to a 2 mL vial, followed by DCM (1 mL). NaIO₄/SiO₂ (226 mg, 3 eq, 14.6% w/w, prepared according to the article published by Zhong, Y-L and Shing, T. K. M. J. Org. Chem., 1997, 62 (8), 2622-2624) was added, and the reaction mixture was stirred for 1 h. The reaction mixture was filtered, and the solid was washed with EtOAc (20 mL×3). The filtrate was concentrated and purified by silica gel column chromatography (0-100% gradient of 80% DCM/20% MeOH/0.5% aqueous NH₄OH in DCM), giving the title compound (21 mg, 68%) as a white foam. MS (ESI+) m/z. 608.15 [M+H]. ¹H NMR (400 MHz, Chloroform-d) δ 9.15 (d, 1H), 7.98-7.96 (m, 2H), 7.49-7.45 (m, 1H), 7.37-7.34 (m, 2H), 5.01-4.97 (m, 1H), 4.74-4.72 (m, 1H), 3.70 (m, 1H), 3.52-3.44 (m, 2H), 3.41 (s, 1H), 3.14-3.03 (m, 1H), 2.96 (s, 3H), 2.89-2.83 (m, 1H), 2.34-2.29 (m, 1H), 2.22 (s, 6H), 2.12-2.05 (m, 1H), 1.75-1.69 (m, 2H), 1.62-1.56 (m, 1H), 1.44-1.36 (m, 1H), 1.32 (s, 9H), 1.20 (d, 3H), 1.17 (s, 3H), 1.08 (d, 3H), 0.88 (d, 3H), 0.59 (d, 3H).

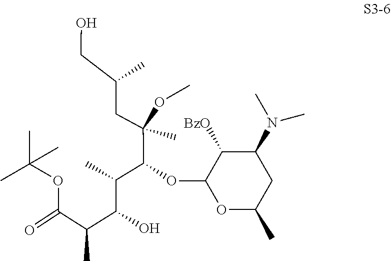

(3R,4S,6R)-2-(((2R,3S,4S,5R,6R,8R)-1-(tert-butoxy)-3,9-dihydroxy-6-methoxy-2,4,6,8-tetramethyl-1-oxononan-5-yl)oxy)-4-(dimethylamino)-6-methyltetrahydro-2H-pyran-3-yl benzoate (S3-6). S4-12 (100 mg, 0.16 mmol) was added to a 20 mL vial, followed by EtOH (1 mL). NaBH₄ (12.4 mg, 2 eq) was added, and the mixture was stirred for 15 min. The reaction mixture was concentrated and was purified by silica gel column chromatography (0-100% gradient of 80% DCM/20% MeOH/0.5% aqueous NH₄OH in DCM), giving the title compound (88 mg, 88%) as a white foam. MS (ESI+) m/z: 610.30 [M+H].

### Conversion of S1-2 to S5-7 Using Ozonolysis Route 4

An additional process for preparing the azalide building block S5-7 via ozonolysis route 4 is depicted in Scheme 5. Protection of S1-2 provided S5-1. Conversion of S5-1 to S5-7 was accomplished via two routes. In the first, ozonolysis of S5-1, followed by reduction, hydrolysis, and esterification with benzyl bromide (protection) provided ester S5-5. Periodate oxidation of S5-5, followed by reduction of S5-6, provided S5-7. Alternatively, reduction of S5-1, followed by ozonolysis, hydrolysis and esterification with benzyl bromide (protection), provided S5-11. Periodate oxidation of S5-11 provided S5-6 and subsequently S5-7 upon reduction.

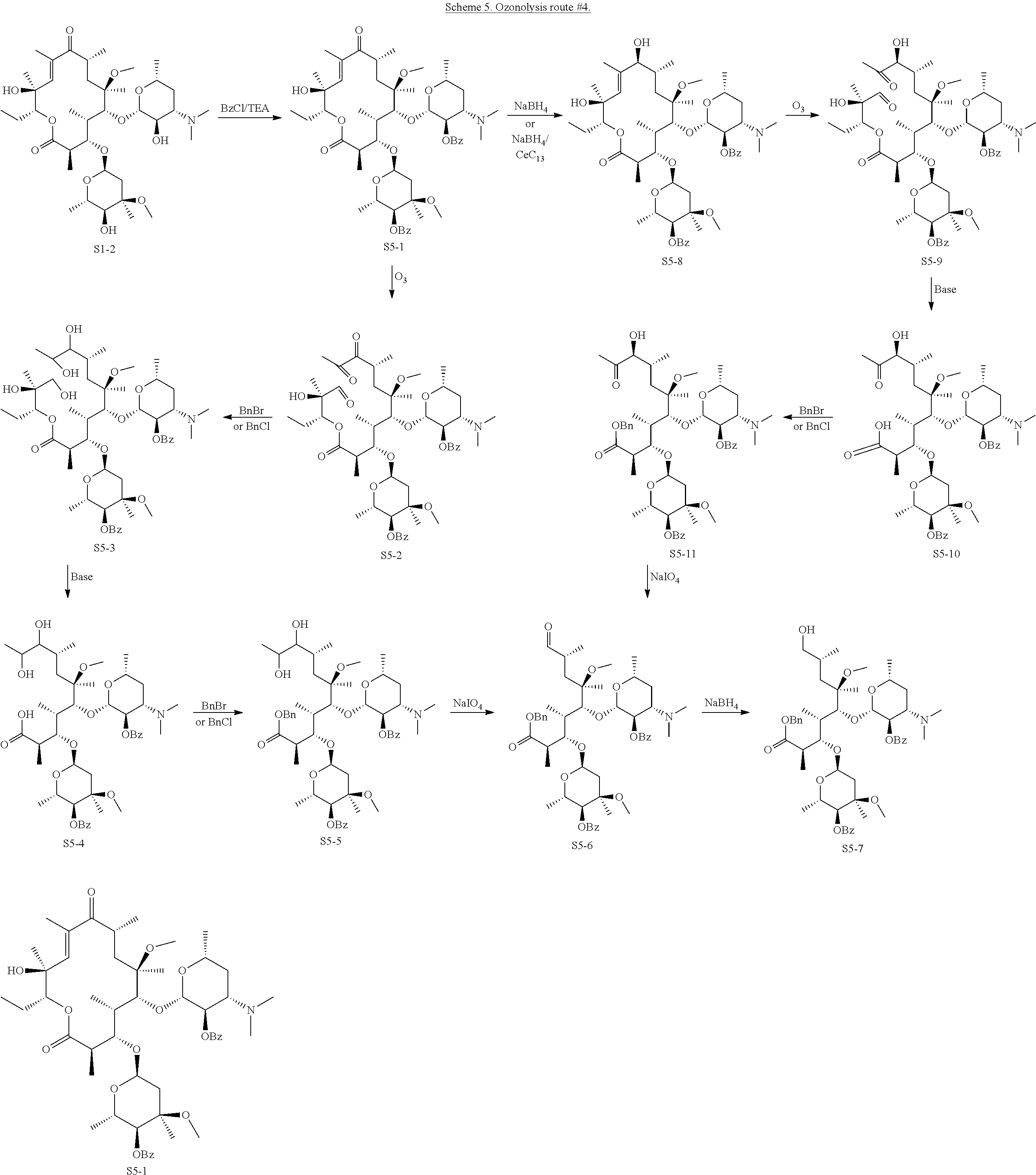

(2S,3S,4R,6R)-6-(((3R,4S,5S,6R,7R,9R,13S,14R,E)-6-(((2S,3R,4S,6R)-3-(benzoyloxy)-4-(dimethylamino)-6-methyltetrahydro-2H-pyran-2-yl)oxy)-14-ethyl-13-hydroxy-7-methoxy-3,5,7,9,11,13-hexamethyl-2,10-dioxooxacyclotetradec-1l-en-4-yl)oxy)-4-methoxy-2,4-dimethyltetrahydro-2H-pyran-3-yl benzoate (S5-1). A 500 mL three-neck round-bottom flask equipped with a stir bar, temperature probe, and nitrogen inlet and outlet was charged with S1-2 (30 g, 41 mmol), followed by anhydrous DCM (150 mL). The solution was cooled with an ice-water bath, and DMAP (8.7 g, 71.2 mmol, 1.74 eq) and triethylamine (6.22 g, 61.5 mmol, 1.5 eq) were added. Benzoyl chloride (11.8 g, 108 mmol, 2.05 eq) was added dropwise while keeping the internal temperature below 25° C. The addition was completed in about 15 min. The reaction mixture was than stirred at room temperature for 16 h. The solution was concentrated, diluted with IPAc (300 mL), washed with H₂O (50 mL), 50% saturated K₂CO₃ (50 mL), and 50% saturated NaH₂PO₄ (2×50 mL), dried over Na₂SO₄, and concentrated to a thick oil. The product was crystallized from EtOH/H₂O (2:1), yielding the title compound (29.5 g, 79%) as a white solid. MS (ESI+) m/z: 938.30 [M+H].

### Large Scale Procedures for S5-1

Method 1. S1-2 (745 g, 1.02 mol) were charged to a 4 L, three neck jacketed reactor equipped with an overhead stirrer. Dichloromethane (2.5 L) was then added, cooled to 0° C., then the reactor was equipped with a thermometer and nitrogen flow. To the orange solution was added DMAP (124 g, 1.02 mol, 1 equiv.) followed by TEA (212 mL, 1.53 mol, 1.5 equiv.). BzCl (241 mL, 2.09 mol, 2.05 equiv.) was added slowly while keeping the internal temperature below 25° C. The reaction mixture was stirred at room temperature for 16 h. The solution was then washed with water (1.5 L), followed by NaH₂PO₄ (1.5 L), then 50% brine (1 L). The washed solution was dried over Na₂SO₄, filtered, and concentrated to near dryness. The residue was crystalized from MTBE/heptane (5:1, 1.5 L/0.3 L), yielding the title compound (666.9 g, 70%).

Method 2. A 400 L jacketed reactor was equipped with an overhead stirrer, thermometer and nitrogen inlet and outlet. S1-2 (7.5 kg) were charged, followed by dichloromethane (35 kg), TEA (2.9 kg) and DMAP (0.9 kg). The reaction mixture was cooled to 5-10° C. BzCl (4.5 kg) was charged slowly to the reactor over 1-1.5 h while keeping the internal temperature below 15° C. The reaction mixture was stirred at 23-27° C. for 16-18 h, then 35-40° C. for 5 h. After cooling to 0-15° C., water (5.0 kg) was charged slowly to the mixture over 1-1.5 h while keeping the internal temperature below 15° C. The agitation was continued for 30 min at 0-15° C. 40% NaH₂PO₄ (12.5 kg) was slowly added while keeping the internal temperature below 25° C. After stirring for 20-30 min at 0-15° C., the mixture was left standing until two clear layers formed. The organic phase was separated and washed with 40% NaH₂PO₄ (12.5 kg). The resulting organic phase was washed with 25% K₂CO₃ (10.0 kg) twice. The organic phase was then diluted with EtOH (18 kg) and concentrated to 3.5-4 volume. This EtOH (18 kg) swap was repeated. EtOH (12 kg) was then added to the organic phase, and the solution was heated to 40-45° C. Water (45 kg) was slowly to the mixture with agitation at this temperature for 1 h, then 20-25° C. for 2-3 h. The white solid was filtered and washed with EtOH/water (1/4) and dried to a consistent weight (8.0 kg, 89%).

Method 3. 49.1 kg S1-2, 230.4 Kg DCM, 21.8 kg TEA and 4.3 kg DMAP were charged into a glass-lined reactor. The temperature was adjusted to −5-5° C. 25.0 kg BzCl was slowly added dropwise into the reaction mixture at −5-5° C. The temperature was adjusted to 15-25° C. and the mixture was agitated for 20-22 hrs. 100.9 kg H₂O were slowly added dropwise to the reaction mixture at approximately 0 to 10° C. NaH₂PO₄ (wt %=20%, aqueous solution) was charged to the reaction mixture which was then agitated for 10-20 min. The aqueous phase was removed. NaH₂PO₄ (wt %=20%, aqueous solution) was charged to the reaction mixture which was then agitated for 10-20 min. The aqueous phase was removed. 35.9 kg K₂CO₃ (wt %=25%, aqueous solution) were charged to the reaction mixture which was then agitated for 10-20 min. The aqueous phase was removed. 35.9 kg K₂CO₃ (wt %=25%, aqueous solution) were charged to the reaction mixture which was then agitated for 10-20 min. The aqueous phase was removed. 110 kg MTBE were charged to the organic phase. The organic phase was then concentrated to 5˜6 volumes under vacuum (approximately −60 kPa to −100 kPa) at 30-40° C. 109.7 kg MTBE were charged to the organic phase. The organic phase was then concentrated to 5˜6 volumes under vacuum (approximately −60 kPa to −100 kPa) at 30-40° C. 73.0 Kg MTBE to the residue. The mixture was agitated at approximately 30 to 40° C. for approximately 1-2 hrs. 339.6 kg n-heptane were then added slowly dropwise to the reaction mixture, which was then agitated at 30-40° C. for 1-2 hrs and then centrifuged. The resulting centrifugate cake was then washed with a mixture of MTBE (109.6 Kg)/n-heptane (101.4 Kg) solution and dried at 45-50° C. for 11-13 hrs. 59.48 Kg of S5-1 was obtained as a white solid, 94.2% yield.

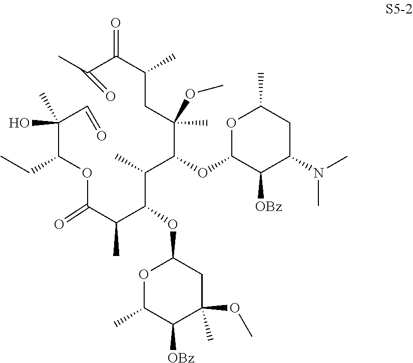

(2S,3S,4R,6R)-6-(((2R,3S,4S,5R,6R,8R)-5-(((2S,3R,4S,6R)-3-(benzoyloxy)-4-(dimethylamino)-6-methyltetrahydro-2H-pyran-2-yl)oxy)-1-(((2R,3R)-2-hydroxy-2-methyl-1-oxopentan-3-yl)oxy)-6-methoxy-2,4,6,8-tetramethyl-1,9,10-trioxoundecan-3-yl)oxy)-4-methoxy-2,4-dimethyltetrahydro-2H-pyran-3-yl benzoate (S5-2). Prepared according to the methods of S4-4 from S5-1 (14.4 g, 15.3 mmol), giving the title compound (14.3 g) as a white foam, which was used in the next step without further purification. MS (ESI+) m/z: 970.39 [M+H]⁺.

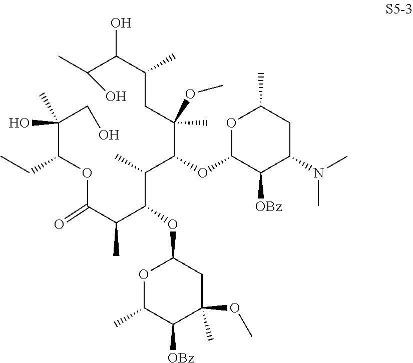

(2S,3S,4R,6R)-6-(((2R,3S,4S,5R,6R,8R)-5-(((2S,3R,4S,6R)-3-(benzoyloxy)-4-(dimethylamino)-6-methyltetrahydro-2H-pyran-2-yl)oxy)-1-(((2S,3R)-1,2-dihydroxy-2-methylpentan-3-yl)oxy)-9,10-dihydroxy-6-methoxy-2,4,6,8-tetramethyl-1-oxoundecan-3-yl)oxy)-4-methoxy-2,4-dimethyltetrahydro-2H-pyran-3-yl benzoate (S5-3). Prepared by NaBH₄ reduction according to the methods of S4-5 starting from S5-2 (15 g, 15.4 mmol), giving the title compound (14 g) as a white foam, which was used in the next step without further purification. MS (ESI+) m/z: 976.19 [M+H]⁺.

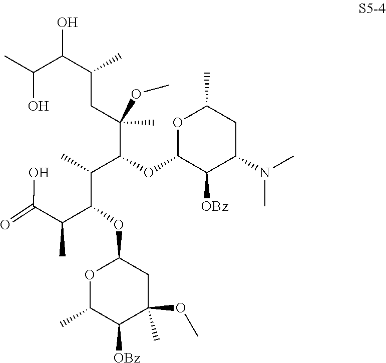

(2R,3S,4S,5R,6R,8R)-5-(((2S,3R,4S,6R)-3-(benzoyloxy)-4-(dimethylamino)-6-methyltetrahydro-2H-pyran-2-yl)oxy)-3-(((2R,4R,5S,6S)-5-(benzoyloxy)-4-methoxy-4,6-dimethyltetrahydro-2H-pyran-2-yl)oxy)-9,10-dihydroxy-6-methoxy-2,4,6,8-tetramethylundecanoic acid (S5-4). Prepared according to the methods of S4-10 starting from S5-3 (2.25 g, 2.3 mmol), giving the title compound (1.4 g, 71%) as a white foam. MS (ESI+) m/z: 860.15 [M+H]⁺.

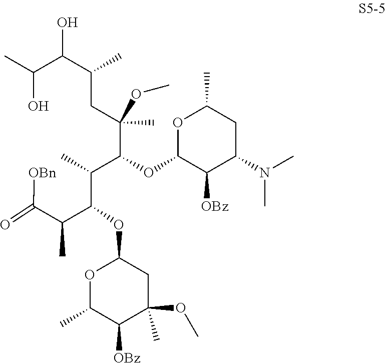

(2S,3S,4R,6R)-6-(((2R,3S,4S,5R,6R,8R)-5-(((2S,3R,4S,6R)-3-(benzoyloxy)-4-(dimethylamino)-6-methyltetrahydro-2H-pyran-2-yl)oxy)-1-(benzyloxy)-9,10-dihydroxy-6-methoxy-2,4,6,8-tetramethyl-1-oxoundecan-3-yl)oxy)-4-methoxy-2,4-dimethyltetrahydro-2H-pyran-3-yl benzoate (S5-5). S5-4 (8.7 g, 10.1 mmol) was dissolved in CH₃CN (90 mL) in a 500 mL round-bottom flask. DBU (1.65 mL, 11.1 mmol, 1.1 eq) and benzyl bromide (1.38 mL, 11.6 mmol, 1.15 eq) were added, and the reaction mixture was stirred at rt for 16 h. The mixture was concentrated under reduced pressure, and the residue was re-dissolved in IPAC (120 mL). This was washed with 13% NaCl (50 mL), was dried over Na₂SO₄, and was concentrated to give the title compound (8.8 g) as a white foam. MS (ESI+) m/z: 950.18 [M+H]⁺.

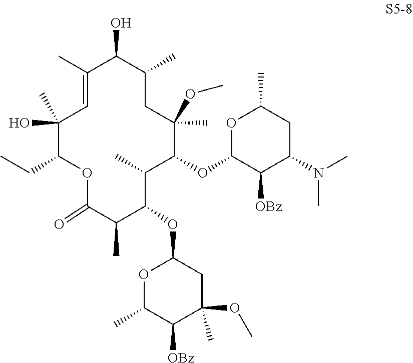

(2S,3S,4R,6R)-6-(((3R,4S,5S,6R,7R,9R,10S,13S,14R,E)-6-(((2S,3R,4S,6R)-3-(benzoyloxy)-4-(dimethylamino)-6-methyltetrahydro-2H-pyran-2-yl)oxy)-14-ethyl-10,13-dihydroxy-7-methoxy-3,5,7,9,11,13-hexamethyl-2-oxooxacyclotetradec-11-en-4-yl)oxy)-4-methoxy-2,4-dimethyltetrahydro-2H-pyran-3-yl benzoate (S5-8). Prepared according to the methods of S4-3 starting from S5-1 (10.5 g, 12.2 mmol) in EtOH/THF (1/1), yielding the title compound (11.5 g) as a white foam, which was used in the next step without further purification. MS (ESI+) m/z: 940.30 [M+H]⁺.

### Large Scale Procedure for S5-8

Method 1. S5-1 (315.56 g) was added to a 5 L jacketed reactor equipped with a mechanical stirrer followed by THF (1.39 L) and MeOH (1.39 L) under nitrogen balloon protection. The mixture was stirred at approximately 0-3° C. to form a yellow solution. CeCl₃·7H₂O (275.71 g, 2.2 eq) was added to the solution with stirring until all solids had dissolved. NaBH₄ (27.99 g, 2.2 eq) was added portionwise and then the mixture was stirred at approximately 2-5° C. for 50 min, at which time HPLC showed >99% conversion. H₂O (316 mL) was added to the reaction mixture slowly, and the pH of the mixture was adjusted with 3N HCl to pH of approximately 5-6 with some white solid appearing. The mixture was concentrated to remove most of the THF and MeOH. DCM (1.35 L) was then added, and the reaction mixture was washed with water (315 mL×2). The aqueous phase was separated, and the organic phase was concentrated to give crude S5-8 as yellowish oil. MTBE (945 mL, 3 vol) was added to reaction mixture with stirring to form a white turbid mixture. Hexanes (1.89 L, 6 vol) was then added slowly and the mixture was stirred for 1 h. The white solid was filtered and washed with solvent (500 mL, MTBE/hexanes (v/v)=1/2) and then dried in high vacuum at 50° C. for 16 h to yield S5-8 (287 g, 90.6% yield).

Method 2. 65.0 kg S5-1, 578.6 Kg THF and 1026.8 kg MeOH were charged into a glass-lined reactor (3000 L). The mixture was agitated at 100 rpm at 15-25° C. for 10-20 mins. 57.0 kg CeCl₃·7H₂O was then charge4d into the reaction mixture. The mixture was agitated at 15-25° C. for 10-20 mins. The temperature was then adjusted to 0-10° C. 6.3 kg NaBH₄ was then added portionwise to the reaction mixture while keeping the internal temperature at 0-10° C. The temperature was adjusted to 15-25° C. and the mixture was agitated for 1-2 hrs. 130.0 kg H₂O were then slowly added to the reaction mixture while keeping the internal temperature at 0-10° C. The temperature was adjusted to 15-25° C., and the mixture was agitated for 10-20 mins. 23.9 kg HCl (wt %=10.5%, aqueous solution) was then added to the reaction mixture, which was agitated for 10-20 mins, and the pH was adjusted to 5-6. 325.0 kg H₂O were then charged into the reaction mixture. The mixture was then concentrated to 3-4 volumes under vacuum at 35-45° C. to remove most of the THF and MeOH. 585.0 Kg DCM was then charged into the concentrated reaction mixture. The resulting mixture was agitated at 15-25° C. for 20-40 mins, and then allowed to settle at 15-25° C. for 20-40 mins. The organic and aqueous layers were separated. 429. 1.0 Kg DCM were charged into the aqueous layer. The mixture was agitated at 15-25° C. for 20-40 mins, and then allowed to settle for 20-40 mins at 15-25° C. The organic and aqueous layers were separated. The organic layers were combined. 130.0 kg H₂O were charged into the combined organic layer. The mixture was agitated at 15-25° C. for 20-40 mins. The organic aqueous layers were separated, and the aqueous layer was discarded. 429.0 kg of n-Hexane were charged into the organic layer. The mixture was then concentrated under vacuum at 20-30° C. to 8-10 volumes. 214.5 kg of n-hexane were charged into the mixture. The mixture was then concentrated under vacuum at 20-30° C. to 8-10 volumes, resulting in a white suspension. 215.5 kg of n-hexane were into the mixture. The mixture (a suspension) was then concentrated under vacuum at 20-30° C. to 8-10 volumes, resulting in a white suspension. 48.1 kg of MTBE were charged into the white suspension. The temperature was adjusted to 20-30° C. for 2-3 hrs. The white suspension was then filtered to remove the organic layer. The resulting filter cake was then washed with 133.3 kg n-hexane. The filtrate was discarded. The filter cake was slurried with MeOH: 51.8 kg (0.796 w/w), MTBE: 167.2 kg (2.57 w/w) and n-Hexane: 86.7 kg (1.33 w/w) at 35-45° C. The slurry was stirred at 35-45° C. for 1-2 h, and at 0-10° C. for 1-2 h. The white suspension was filtered to remove organic layer. The resulting filter cake was washed with 115.2 kg n-hexane. The filter cake was then dried under vacuum at 45-55° C. for 15-17 hrs. S5-8 was obtained as a white solid, 55.64 kg, 85.42% yield, 92.3% purity.

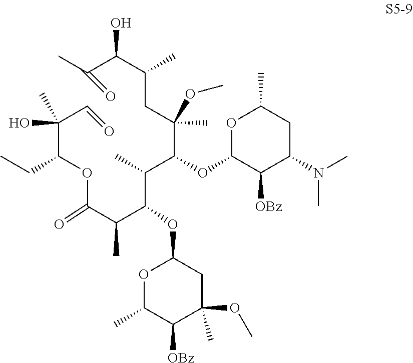

(2S,3S,4R,6R)-6-(((2R,3S,4S,5R,6R,8R,9S)-5-(((2S,3R,4S,6R)-3-(benzoyloxy)-4-(dimethylamino)-6-methyltetrahydro-2H-pyran-2-yl)oxy)-9-hydroxy-1-(((2R,3R)-2-hydroxy-2-methyl-1-oxopentan-3-yl)oxy)-6-methoxy-2,4,6,8-tetramethyl-1,10-dioxoundecan-3-yl)oxy)-4-methoxy-2,4-dimethyltetrahydro-2H-pyran-3-yl benzoate (55-9). Prepared according to the methods of S4-4 starting from S5-8 (11.4 g, 12.2 mmol), yielding the title compound (12.2 g) as a white foam, which was used in the next step without further purification. MS (ESI+) m/z: 972.39 [M+H]⁺.

Prepared according to the methods of S4-4 starting from S5-8 (30.0 g, 32.0 mmol), using 3.6 equivalents of TFA and replacing Me₂S with triphenylphosphine (1.2 equivalents), yielding the crude title compound (45 g) as a white foam, which was used in the next step without further purification.

Prepared according to the methods of S4-4 starting from S5-8 (0.5-30 g), while 2.4-4.8 equivalents of TFA were used and the reactions were quenched with reductants including triphenylphosphine, HOAc/Zn, trimethoxyphosphine and triethoxyphosphine. The crude title compound was used in the next step without further purification.

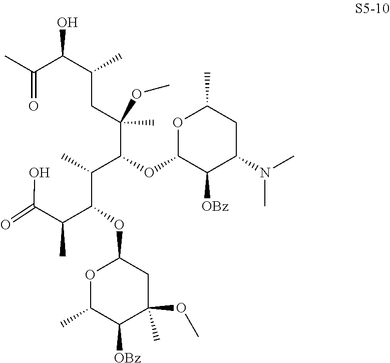

(2R,3S,4S,5R,6R,8R,9S)-(((2S,3R,4S,6R)-3-(benzoyloxy)-4-(dimethylamino)-6-methyltetrahydro-2H-pyran-2-yl)oxy)-3-(((2R,4R,5S,6S)-5-(benzoyloxy)-4-methoxy-4,6-dimethyltetrahydro-2H-pyran-2-yl)oxy)-9-hydroxy-6-methoxy-2,4,6,8-tetramethyl-10-oxoundecanoic acid (S5-10). Prepared according to the methods of S4-10 starting from S5-9 (11.8 g, 12.2 mmol), yielding the title compound (11.5 g) as a white foam. MS (ESI+) m/z: 858.15 [M+H]⁺; ¹H NMR (400 MHz, Chloroform-d) δ 8.06-8.02 (m, 4H), 7.64-7.54 (m, 2H), 7.50-7.43 (m, 4H), 5.79 (s, 1H), 5.13-5.08 (m, 1H), 4.95-4.90 (m, 2H), 4.85-4.81 (m, 1H), 4.55-4.47 (m, 1H), 3.96-3.89 (m, 1H), 3.77 (d, 1H), 3.72 (d, 1H), 3.55 (s, 3H), 3.17 (s, 3H), 3.07-2.94 (m, 1H), 2.81-2.77 (m, 1H), 2.54-2.50 (m, 1H), 2.35 (s, 6H), 2.28-2.24 (m, 1H), 1.82 (s, 3H), 1.76-1.71 (m, 2H), 1.65-1.60 (m, 2H), 1.36 (s, 3H), 1.27 (m, 4H), 1.22 (s, 3H), 1.17 (d, 3H), 1.15 (d, 3H), 0.98 (d, 3H), 0.84 (t, 3H), 0.75 (d, 3H).

Method 2. S5-9 (100 g) was stirred in CH₃CN (500 mL) at 25-26° C. until all solids dissolved. H₂O (500 mL) was added in the reaction mixture. LiOH H₂O (4.82 g, 1.1 eq) was added in the mixture at 0-3° C. and stirred at 14-17° C. for 2 h. The reaction mixture was concentrated to remove most of the CH₃CN. MTBE (50 mL) was added and stirred for 10 min and the two phases were separated. This MTBE extraction was repeated. The aqueous phase was separated and concentrated to remove most MTBE dissolved in water. Aqueous 3N HCl was added to aqueous phase by adjusting the pH to 6-7 to until the phase was turbid. The white solid was filtered and dried under high vacuum at 45-46° C. for 16 h to provide the title compound (57.18 g, 64.4% yield).

Method 3. S5-9 (41.2 g) was dissolved in CH₃CN (200 mL), followed by water (136 mL). The mixture was cooled to 0-5° C. Aqueous NaOH solution (1 N, 64 mL) was added, and the resulting mixture was stirred at ambient temperature for 1 h. The reaction mixture was concentrated under reduced pressure to remove most of the CH₃CN (approximately 200 mL) to leave a viscous residue (approximately 240 g). Water (100 mL) was added and the diluted mixture was extracted with MTBE (200 mL). The aqueous phase was neutralized with 3 N HCl (approximately 22 mL) to a pH of approximately 5-7, followed by extraction with DCM (250 mL). The aqueous phase was extracted again with DCM (100 mL) and the combined organic phases were concentrated. The residue was dissolved in EtOH (160 mL) at 65-75° C., followed by addition of heptane (200 mL). The solution was stirred at ambient temperature for 16 h. The white solid was collected by filtration and dried in high vacuum at 35-45° C. for 16 h to get the title compound (28.6 g, 78.8%).

### Large Scale Procedure for S5-10

Method 4. 5279.2 kg S5-9 solution in DCM (source: 98.0 kg S5-8) was charged into a glass-lined reactor (3000 L). The solution was concentrated under vacuum at 15-25° C. to 3-6 volumes. 363.0 kg ACN were charged into the reactor. The mixture was concentrated under vacuum at 15-25° C. to 3-6 volumes. 160.2 kg ACN were charged into the reactor. The mixture was concentrated under vacuum at 15-25° C. to 4-6 volumes. 121.3 kg ACN were charged into the reactor. The mixture was concentrated under vacuum at 15-25° C. to 3-6 volumes, from which was obtained 425.7 kg of S5-9 as a solution in ACN. 509.6 kg H₂O were charged into the solution. The internal temperature was adjusted to 5-15° C. and 125.3 kg NaOH (wt %=3.85%, aqueous solution) were slowly added while keeping the internal temperature at 5-15° C. The mixture was agitated at 15-25° C. for 2-4 hrs and then concentrated under vacuum at 20-30° C. to 5-7 volumes to remove most of the ACN. 305.6 kg H₂O and 454.5 kg MTBE were then charged into the reactor. The mixture was agitated for 15-25 mins, and then allowed to stand at 10-20° C. for 25-35 mins. The organic and aqueous layers were separated. The organic layer as discarded. 34.3 kg HCl (wt %=9.75%, aqueous solution) were then slowly added to the aqueous layer, and the pH was adjusted to 5-7 at 10-20° C. 944.2 kg DCM was charged into the quenched solution, which was then agitated at 10-20° C. for 25-35 mins. The mixture was then allowed to stand for 25-35 mins. The organic and aqueous layers were separated. 405.7 kg DCM were charged into the aqueous layer and the resulting solution was agitated at 10-20° C. for 25-35 mins, and then allowed to stand at 10-20° C. for 25-35 mins. The organic aqueous layers were separated. The combined the organic layers were concentrated under vacuum at 25-35° C. to 2-4 volumes in a reactor (3000 L). 241.5 kg EtOH were charged into the reactor. The mixture was concentrated under vacuum at 25-35° C. to 2-4 volumes, which resulted in a white suspension. 161.4 kg EtOH was added to the mixture. The mixture was concentrated under vacuum at 25-35° C. to 2-4 volumes, which resulted in a white suspension. 40.6 kg EtOH was charged into the suspension, and then agitated at 55-65° C. for 10-40 mins. 349.2 kg n-hexane were then charged into the white suspension. The temperature was adjusted to 55-65° C. for 2-3 hrs. The internal temperature was adjusted to 5-15° C., and the mixture was agitated for 2-3 hrs. The white suspension was filtered to provide a wet cake. The cake was washed with 349.5 kg n-hexane. The filtrate was discarded. The cake was dried under vacuum at 35-45° C. for 15-24 hrs to provide S5-10 as a white solid, 54.05 kg.

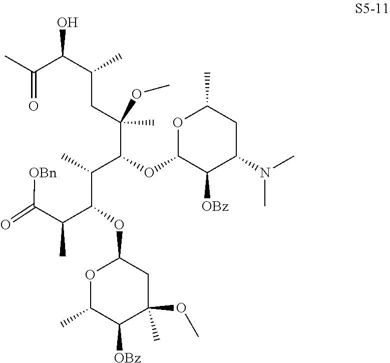

(2S,3S,4R,6R)-6-(((2R,3S,4S,5R,6R,8R,9S)-5-(((2S,3R,4S,6R)-3-(benzoyloxy)-4-(dimethylamino)-6-methyltetrahydro-2H-pyran-2-yl)oxy)-1-(benzyloxy)-9-hydroxy-6-methoxy-2,4,6,8-tetramethyl-1,10-dioxoundecan-3-yl)oxy)-4-methoxy-2,4-dimethyltetrahydro-2H-pyran-3-yl benzoate (S5-11). Prepared according to the methods of S5-5 starting from S5-10 (1.6 g, 1.9 mmol). Purification by silica gel column (0-100% gradient of 80% DCM/20% MeOH/0.5% aqueous NH₄OH in DCM) gave the title compound (1.0 g, 57%) as a white foam. MS (ESI+) m/z: 948.23 [M+H]+; ¹H NMR (400 MHz, Chloroform-A) δ 8.07-8.02 (m, 2H), 8.02-7.95 (m, 2H), 7.65-7.56 (m, 1H), 7.52 (td, 1H), 7.46 (t, 2H), 7.40 (t, 2H), 7.34 (s, 5H), 5.18-4.96 (m, 3H), 4.91 (d, 1H), 4.86-4.77 (m, 2H), 4.47 (dq, Hz, 1H), 4.08 (d, 1H), 3.94 (t, 1H), 3.78 (t, 1H), 3.75-3.69 (m, 1H), 3.67 (d, 1H), 3.38 (d, 3H), 3.09 (s, 3H), 2.99-2.85 (m, 1H), 2.75 (qd, 1H), 2.32 (s, 6H), 2.14 (s, 3H), 2.05 (dd, 1H), 1.70 (dd, 3H), 1.49-1.40 (m, 1H), 1.37 (d, 1H), 1.31 (dd, 1H), 1.26 (s, 3H), 1.18 (d, 4H), 1.12 (d, 6H), 1.01 (d, 3H), 0.96 (d, 3H), 0.67 (d, 3H).

S5-10 (24.5 g, 28.5 mmol) was dissolved in DMF (anhydrous, 100 mL) followed by addition of K₂CO₃ (4.72 g, 34.2 mmol) and BnCl (3.92 mL, 34.2 mmol). The mixture was heated at 40° C. under nitrogen for 16 h. In a separate flask, 75 mL of brine was diluted with 425 mL of deionized water. To this aqueous solution was slowly added the DMF solution with vigorous stirring at rt. The resulting mixture was stirred for an additional 16 h. The white solid was collected by filtration and dried under high vacuum at 35-45° C. for 16 h to get the title compound (26.5 g, 98.1%).

S5-10 (65 g) was dissolved in DCM (650 ml) in a 5 L jacketed reactor and stirred at 24-25° C. under nitrogen. Et-N (15.33 g), BnOH (9.01 g) and 2,4,6-trichlorobenzoyl chloride (22.17 g) were added and the resulting mixture was stirred at 24-25° C. for 30 min. DMAP (1.30 g) was added and the resulting reaction mixture was stirred at 24-25° C. for 1 h. The reaction mixture was concentrated to remove most of the DCM and then was diluted with MTBE (975 mL). The organic phase was washed with aqueous NaOH (1 N, 2×264 mL), aqueous NaH₂PO₄ (50%, 325 mL) and aqueous 13% NaCl (325 mL). The organic phase was concentrated under reduced pressure at 35-36° C. to the title compound as a white foam (75.7 g), which was used in the next step without further purification.

### Large Scale Procedure for S5-11

53.5 kg S5-10, 201.9 kg DMF and 11.3 kg K₂CO₃ were charged into a glass-lined reactor (1000 L). 9.2 kg BnCl were slowly added dropwise to the reaction mixture, keeping internal temperature at 25-35° C. The internal temperature was adjusted to 30-45° C. The mixture was agitated for 17-19 hrs. 540.0 kg H₂O was then slowly added dropwise to the mixture, which was then agitated at 15-25° C. for 5-7 hrs, resulting in a white suspension. The white suspension was filtered to remove the organic layer. The cake was washed portionwise with 1080.0 kg H₂O. The filtrate was discarded. The filtrate was dried by filter dryer at 0-5° C. 44.0 kg S5-11 was obtained as a white solid, containing approximately 7% DMF. 44.0 kg S5-11 and 220 kg H₂O were charged into a glass-lined reactor (300 L). The mixture was stirred for 15-25° C. at 0-5° C. The mixture was filtered to remove the water layer. The cake was washed portionwise with 1080.0 kg H₂O. The solid was dried under vacuum with nitrogen flow at 0-5° C. for 98 h by filter dryer. 41.3 kg S5-11 was obtained as a white solid.

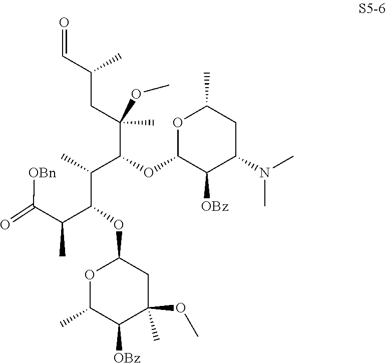

(2S,3S,4R,6R)-6-(((2R,3S,4S,5R,6R,8R)-5-(((2S,3R,4S,6R)-3-(benzoyloxy)-4-(dimethylamino)-6-methyltetrahydro-2H-pyran-2-yl)oxy)-1-(benzyloxy)-6-methoxy-2,4,6,8-tetramethyl-1,9-dioxononan-3-yl)oxy)-4-methoxy-2,4-dimethyltetrahydro-2H-pyran-3-yl benzoate (S5-6). Prepared according to the methods of S4-12 starting from S5-5 or S5-11 yielded the title compound as a white foam. MS (ESI+) m/z: 904.11 [M+H]⁺; ¹H NMR (400 MHz, Chloroform-d) δ 9.27 (d, 1H), 8.08-8.01 (m, 4H), 7.62-7.51 (m, 3H), 7.48-7.41 (m, 4H), 7.38-7.34 (m, 4H), 5.13 (s, 2H), 5.11-5.08 (m, 2H), 5.03-4.99 (m, 1H), 4.94-4.88 (m, 1H), 4.72-4.66 (m, 1H), 3.96-3.94 (m, 1H), 3.67 (d, 1H), 3.38 (s, 3H), 3.32-3.28 (m, 1H), 3.15-3.09 (m, 1H), 3.00 (s, 3H), 2.90-2.78 (m, 1H), 2.34 (s, 6H), 2.19-2.18 (m, 3H), 2.12 (s, 3H), 2.03 (s, 3H), 1.99-1.94 (m, 2H), 1.68-1.51 (m, 4H), 1.26 (s, 3H), 1.24 (s, 3H), 1.20 (d, 3H), 1.13 (t, 3H), 1.00 (d, 3H), 0.98 (d, 3H), 0.73 (d, 3H).

S5-11 (75.70 g) was dissolved in toluene (1.51 L) in a 5 L jacketed reaction bottle and stirred at 24-25° C. NaIO₄/SiO₂ was added and the resulting reaction mixture was stirred at 24-25° C. for 1 h. The reaction mixture was filtered through a silica gel plug (SiO₂, 100 g, eluting with EtOAc (227 mL). The organic phase was washed with Na₂SO₃ (60.33 g, 6.0 eq, in 200 mL water) and aqueous NaCl (150 mL, wt=13% in H₂O). Na₂SO₄ (45 g) was added to the organic phase. After one hour, the organic phase was separated and concentrated under reduced pressure at below 30° C. to yellowish liquid. The yellowish liquid was dissolved in DCM (4×50 mL) and concentrated under reduced pressure 4 times, and then dried under high vacuum at rt for 4 h to the title compound as a white foam (72.3 g), which was used in next step without further purification.

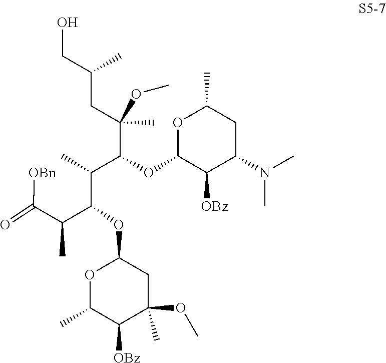

(2S,3S,4R,6R)-6-(((2R,3S,4S,5R,6R,8R)-5-(((2S,3R,4S,6R)-3-(benzoyloxy)-4-(dimethylamino)-6-methyltetrahydro-2H-pyran-2-yl)oxy)-1-(benzyloxy)-9-hydroxy-6-methoxy-2,4,6,8-tetramethyl-1-oxononan-3-yl)oxy)-4-methoxy-2,4-dimethyltetrahydro-2H-pyran-3-yl benzoate (S5-7). Prepared according to the methods of S3-6 (from S4-12 in Scheme 4) starting from S5-6 (7.3 g, 8.1 mmol), yielding the title compound (7.1 g) as a white foam. MS (ESI+) m/z: 906.13 [M+H]^{t}.

S5-3 (72.01 g, 10.55 mmol) was dissolved in THF (720 mL) and cooled to 0-5° C. NMM (4.83 g, 0.6 eq) and isobutyl chloroformate (5.44 g, 0.5 eq) were added and stirred at 0-3° C. for 30 min. MeOH (360 mL) was added to the reaction mixture at 0-3° C. NaBH₄ (6.03 g, 2.0 eq) was then added slowly at 0-10° C. over 30 min. 3 N aqueous HCl was added slowly to the reaction mixture, adjusting the pH to approximately 5-6 at 0-3° C. The solution was concentrated to remove most of the MeOH and THF, and water (144 mL) was added. The solution was extracted with EtOAc (720 mL) and washed with 13% aqueous NaCl (144 mL). Na₂SO₄ (40.0 g) was added to the organic phase. After two hours, the organic phase was separated and concentrated under reduced pressure at 30° C. to give an oil and dried under high vacuum at rt to obtain 73.12 g of white foam. The product was further purified by column chromatography (silica gel, 813 g, 200-300 mesh) using EtOAc/n-Hexane/Et₃N (1/2/0.03) as the eluent to give the title product (44.31 g, 61% yield).

### Synthesis of Azalides Via S3-6 (Schemes 3 and 4)

Scheme 6 depicts an approach to azalide preparation from S3-6. The approach entails oxidation of S3-6 to provide S6-1. Reductive amination of S6-1 with S6-2, followed by ring closure, provides S6-3. Further elaboration of S6-3 includes: N-substitution at N-9a under reductive amination conditions using R₅—CHO or a similar ketone, C-2 alkylation, and deprotection of the C-5 desosamine hydroxyl group to provide the azalide S6-4 wherein R₉ₐ is R₅—CH₂— and R_{2b} is as defined for formula I compounds.

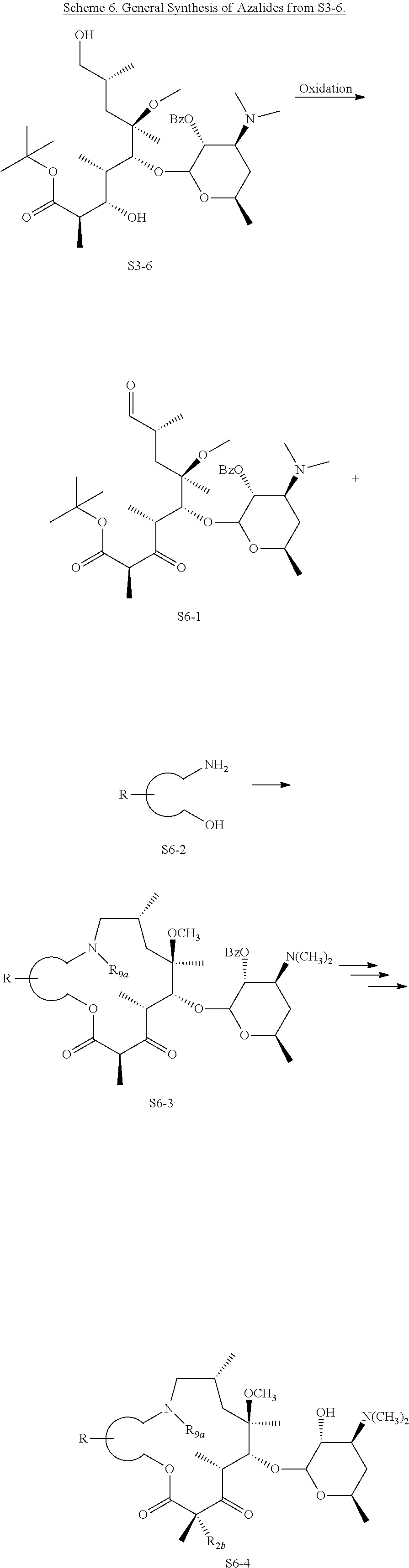

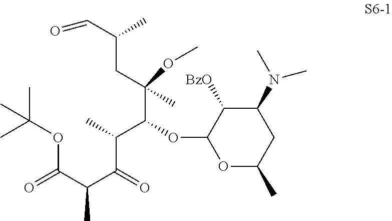

Compound S3-6 (20 mg, 32.7 μmol) in DCM (1 mL) was stirred with Dess Martin reagent (34.6 mg, 81.7 μmol) for 30 min at RT. The mixture was diluted with MTBE (15 mL) and filtered through Celite®. The filtrate was concentrated, and the residue was dried under vacuum to give crude S6-1, which was used directly in the next step. MS (ESI+) m/z: 606.19 [M+H]+

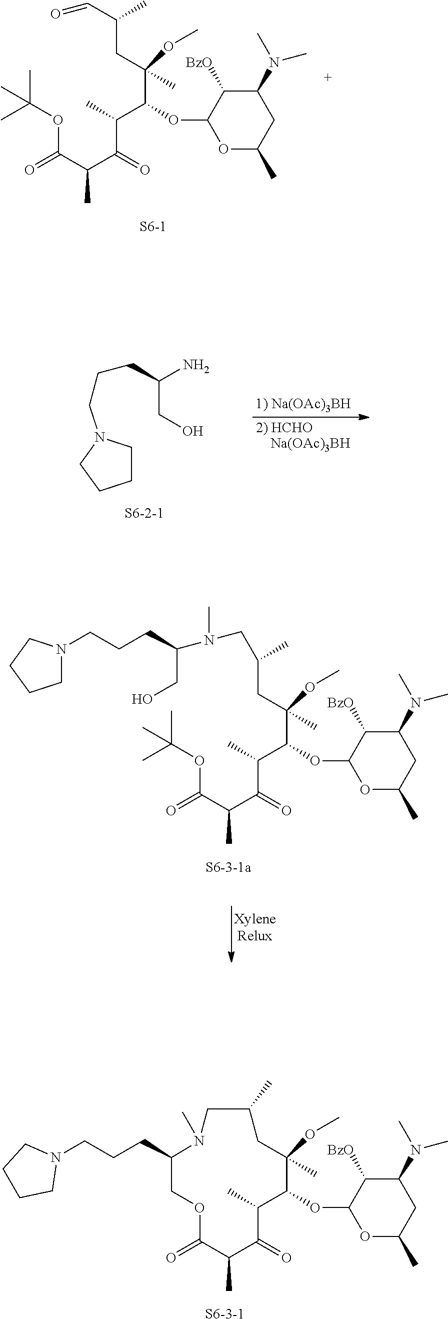

To the crude S6-1 (20 mg, 0.033 mmol) in DCM was added S6-2-1 (7 mg, 0.0395 mmol), AcOH (2 drops) and NaBH(OAc)₃ (13.9 mg, 0.066 mmol). The mixture was stirred at RT for 30 min, then formaldehyde (27 mg, 37% in water, 0.12 mmol) and NaBH(OAc)₃ (28 mg, 0.13 mmol) were added. After stirring for 40 min, the solution was diluted with DCM, washed with sat. NaHCO₃ and water, and dried over Na₂SO₄. The solvent was removed and residue was purified by ISCO to give S6-3-1a, 14 mg, 54%. MS (ESI+) m/z: 776.26 [M+H]⁺ Compound S6-3-1a (10 mg, 0.0128 mmol) was rotovaped with anhydrous toluene (5 mL) and dried under vacuum. The residue was dissolved in m-Xylene (5.5 mL) and bubbled with N₂ for 5 min. The solution was refluxed for 3 h to give S6-3-1. MS (ESI+) m/z: 702.22 [M+H]⁺ An alternative general procedure is further demonstrated in Scheme 7. Starting from S4-6, coupling of S4-6 with S7-1 under reductive amination conditions provides S7-2. N-substitution at the N-9a position under reductive amination conditions using an aldehyde R₅—CHO or similar ketone provides S7-2 wherein R₉ₐ is R₅—CH₂—. Conversion of S7-3 to the acid S7-4, followed by formation of the mixed anhydride and cyclization provides S7-5. Removal of the C-5 cladinose of S7-5 provides S7-6. Oxidation of S7-6 provides S7-7. The process culminates in C-2 alkylation wherein R_{2b} is as defined for compounds of formula I and deprotection of the C-5 desosamine to provide azalide S7-8.

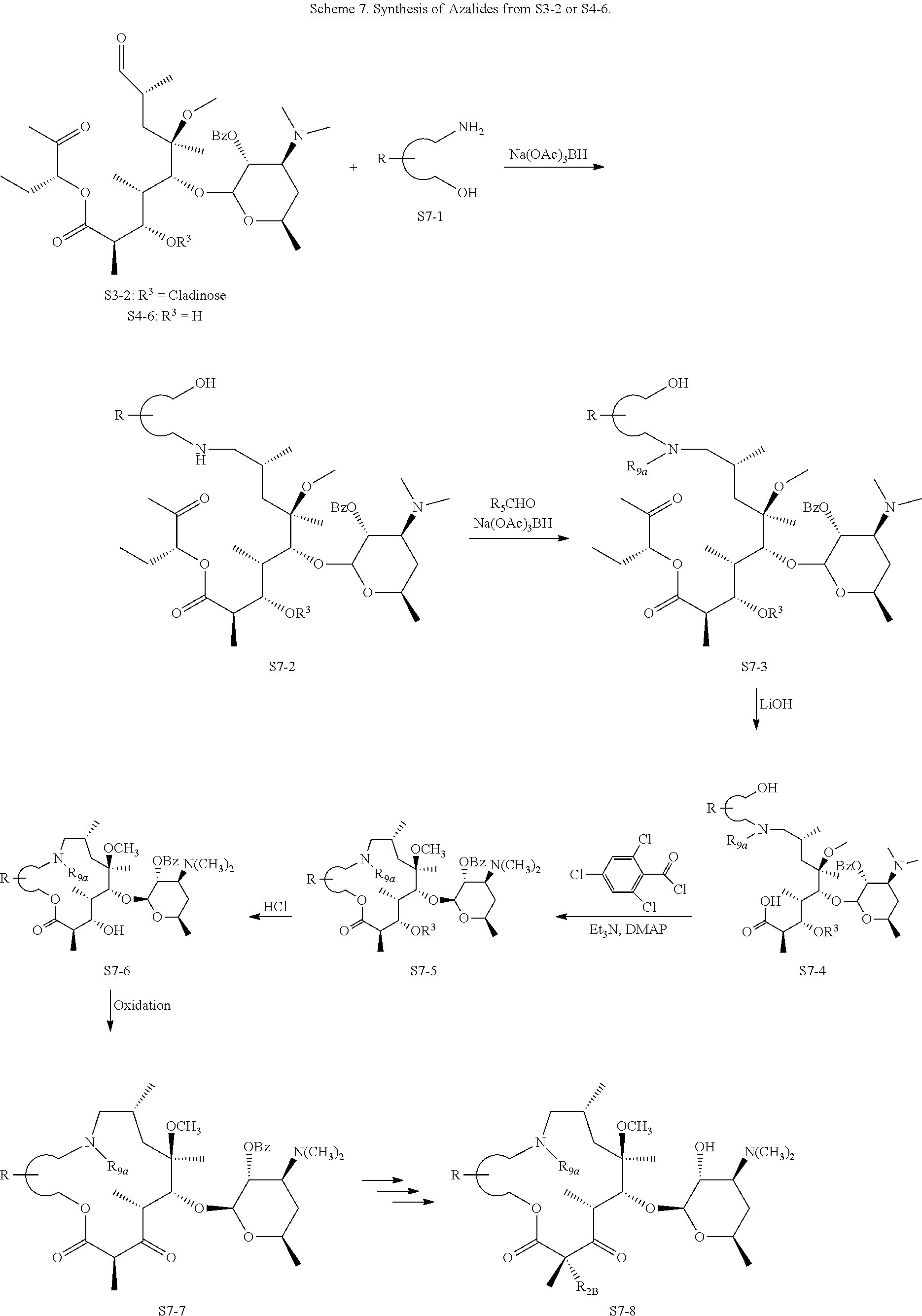

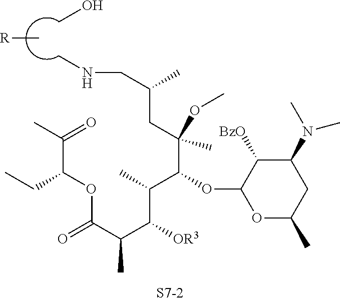

General procedure: S7-1 (1.2 eq) in DCM (0.2 M) was stirred with acetic acid (2 eq) and Na(OAc)₃BH (2 eq) at 0° C. for 10 min and S3-2 or S4-6 (1 eq) was added. The reaction mixture was allowed to warm to rt and stir until complete (30 min to overnight). This solution was used directly in next step.

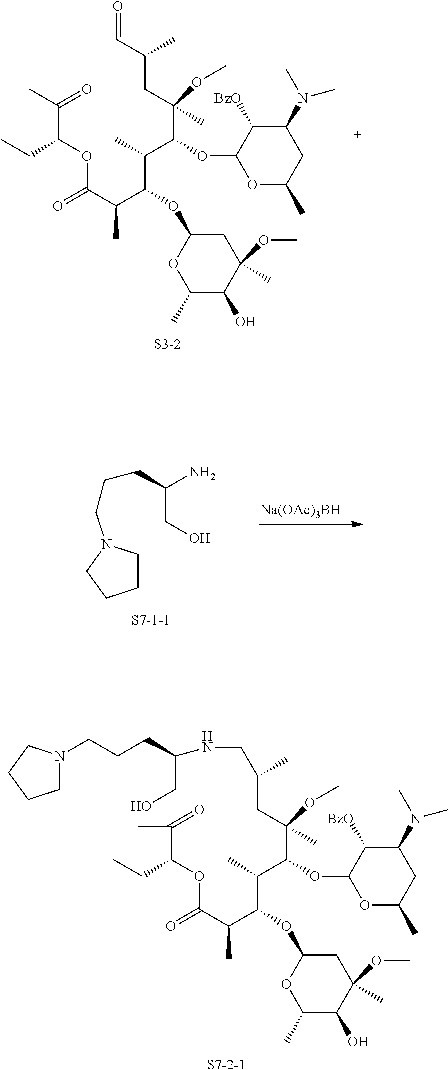

Compound S7-1-1 (377 mg, 2.19 mmol) in DUM (20 mL) was cooled in an acetone-dry ice bath to an internal temperature of −17° C. Acetic acid (0.436 mL, 7.64 mmol) was added under nitrogen, temperature rose to −13° C., then Na(OAc)₃BH (606 mg, 2.86 mmol) was added in one portion. Compound S3-2 (1.52 g, 1.91 mmol) in DCM (13 mL) was added, keeping the internal temperature below −10° C. The reaction mixture was allowed to warm up slowly to 1 PC over 2 h. Another portion of Na(OAc)₃BH (606 mg, 2.86 mmol) was added at this temperature, stirred 30 min, to give complete conversion to S7-2-1, MS (ESI+) m/z: 950.36 [M+H]⁺

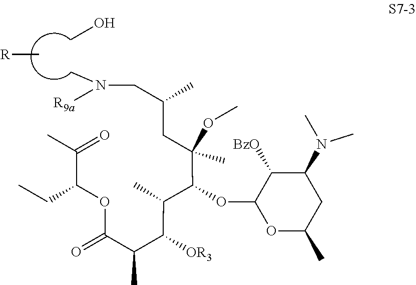

General procedure: To the above solution was added R₅CHO (3 eq) and Na(OAc)₃BH (2 eq). The resulting mixture was stirred for 30 min to overnight. The mixture was diluted with EtOAc (3×DCM volume) and was washed with saturated, aqueous NaHCO₃ and brine. The organic layer was dried over Na₂SO₄, filtered, and concentrated. The material was purified by ISCO (gradient of A: DCM, B: 20% MeOH in DCM with 0.5% NH₄OH) to give the desired compounds.

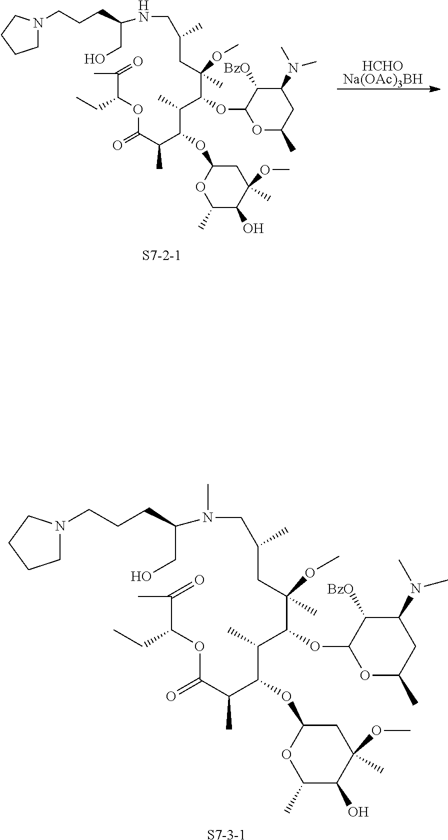

To S7-2-1 in the reaction mixture was added formaldehyde (37% in water, 0.62 mL, 8.29 mmol) and Na(OAc)₃BH (606 mg, 2.86 mmol). The reaction mixture was stirred at RT for 1.5 h. After the addition of saturated NaHCO₃ (30 mL), pH ˜9, the reaction mixture was diluted with IPAc (120 mL), and the organic phase was separated. The water layer was extracted with IPAc (2×40 mL). The combined organic solution was dried over Na₂SO₄, filtered and concentrated. The crude material was purified by ISCO (A: DCM, B. 20% MeOH in DCM with 0.5% NH₄OH), product was out at 100/6 B. This gave 1.47 g (79.8%) of compound S7-3-1 as a white foam. MS (ESI+) m/z: 964.29 [M+H]⁺. ¹H NMR (400 MHz, Chloroform-d) δ 8.04-7.96 (m, 2H), 7.54 (t, 1H), 7.42 (t, 2H), 5.19-5.01 (m, 1H), 4.95-4.85 (m, 1H), 4.79 (d, 1H), 4.73 (d, 1H), 4.13-3.93 (m, 2H), 3.67 (d, 1H), 3.65-3.56 (m, 1H), 3.50 (dd, 1H), 3.38 (s, 3H), 3.34-3.20 (m, 2H), 3.18 (s, 3H), 3.05 (d, 1H), 2.94-2.83 (m, 1H), 2.78 (qd, 1H), 2.65 (tt, 1H), 2.53 (s, 4H), 2.45 (t, 2H), 2.40-2.27 (m, 8H), 2.26 (s, 1H), 2.21 (s, 4H), 2.15 (s, 3H), 2.13-2.04 (m, 1H), 1.88-1.67 (m, 8H), 1.65-1.40 (m, 6H), 1.32 (d, 3H), 1.30-1.18 (m, 10H), 1.15 (d, 3H), 1.10 (q, 1H), 0.93 (t, 6H), 0.74 (d, 3H).

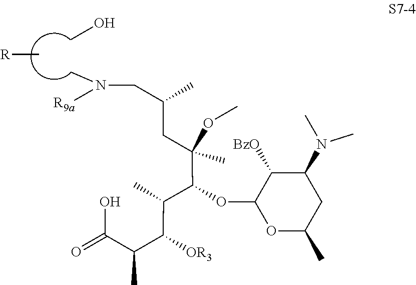

General procedure: A solution of S7-3 (1 eq) in THF/H₂O (3:1, 0.1 M) was stirred with LiOH (5 eq) at rt for 2 h. Water and formic acid (approximately 8 eq) were added to adjust the pH to approximately 6. After the mixture was extracted with DCM two times, the water layer was treated with excess saturated, aqueous NaHCO₃.

For water soluble compounds, the water solution was concentrated and the residue was dried under vacuum. The solid was triturated with DCM three times. The combined DCM solution were concentrated, and the residue was dried under vacuum to give the desired compounds, S7-4.

For non-water soluble compounds, the water layer was extracted with DCM three times. The combined DCM extracts were washed with brine, were dried over Na₂SO₄, were filtered, and were concentrated. The residue was dried under vacuum to give the desired compounds, S7-4.

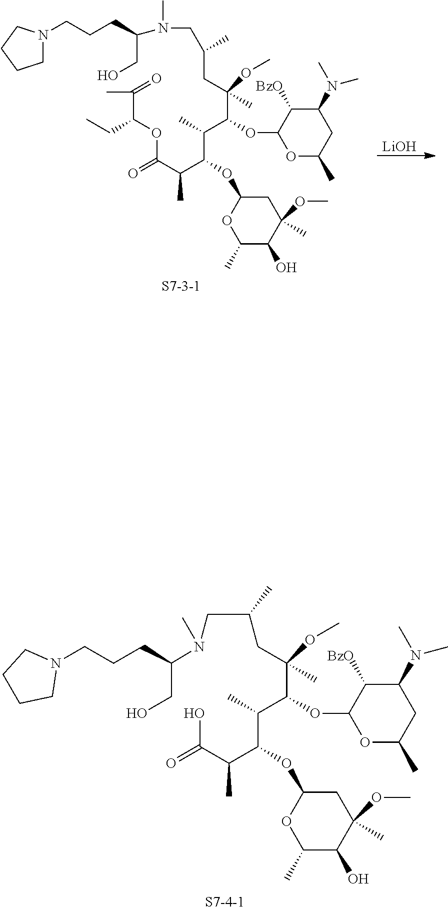

Compound S7-3-1 (1.2 g, 1.24 mmol) in THF (9 mL)/H₂O (3 mL) was stirred with LiOH (148 mg, 6.19 mmol) at RT for 2 h. Adjusted pH to 8-9 by adding slowly HCl (6 mL, 1M, 6 mmol). The mixture was concentrated to dryness under vacuum, and the solid was extracted with DCM (3×15 mL). After DCM was removed, compound S7-4-1 was obtained as a white foam, 1.02 g, 93%. MS (ESI+) m/z: 880.31 [M+H]⁺ ¹H NMR (400 MHz, Chloroform-) S 8.06-7.92 (m, 2H), 7.52 (t, 1H), 7.40 (t, 2H), 5.10-4.95 (m, 1H), 4.88 (d, 1H), 4.69 (d, 1H), 4.06 (dt, 1H), 3.90 (d, 1H), 3.81-3.71 (m, 1H), 3.71-3.62 (m, 1H), 3.62-3.50 (m, 1H), 3.42 (s, 3H), 3.35-3.23 (m, 4H), 3.01 (t, 2H), 2.90-2.56 (m, 7H), 2.48 (dq, 2H), 2.37 (d, 3H), 2.28 (d, 9H), 1.89-1.75 (m, 6H), 1.67-1.49 (m, 5H), 1.41 (d, 1H), 1.35-1.20 (m, 17H), 1.08 (d, 3H), 0.98 (d, 3H), 0.75 (d, 3H).

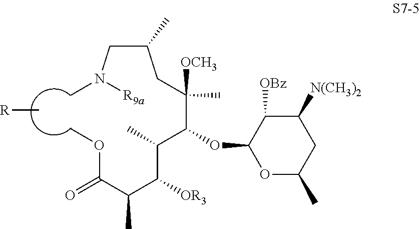

General procedure: To a solution of S7-4 (1 eq) in dry DCM (38 V) was added Et₃N (2 eq) and 1,3,5-trichlorobenzoyl chloride (1.2 eq). After 30 min, DMAP (0.02 eq) was added. The mixture was stirred at rt for 30 min. The solution was washed with saturated, aqueous NaHCO₃ (3 times) and brine (1 times). The DCM layer was concentrated, and the residue was dried under vacuum to give the desired compounds, S7-5.

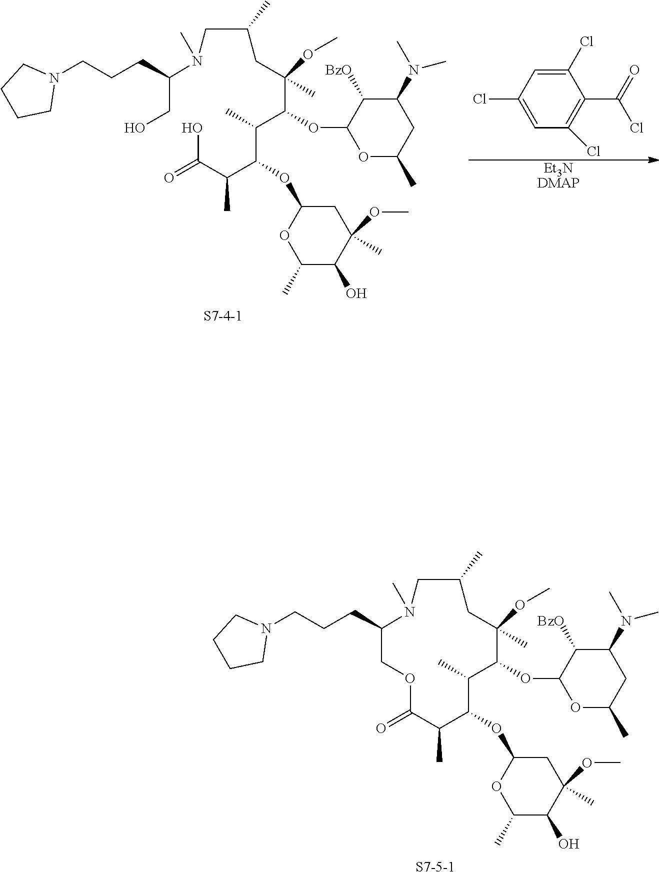

To compound S7-4-1 (500 mg, 0.568 mmol) in dry DCM (19 mL) was added Et₃N (113 mg, 1.12 mmol), then 1,3,5-trichlorobenzoyl chloride (166 mg, 0.681 mmol) in DCM (0.6 mL). After stirring for 30 min, UPLC-MS indicated major product peak (>90%) and minor SM (<5%). DMAP (1.38 mg, 0.0113 mmol) in DCM (0.6 mL) was added and the mixture was stirred at RT for 30 min. The reaction solution was washed with sat. NaHCO₃ (3×20 mL) and brine (20 mL) and was dried over Na₂SO₄. Concentrated and dried under vacuum to give crude compound S7-5-1 as a yellowish foam, about 530 mg, 108%. Major impurity was 1,3,5-trichlorobenzoic acid. Some crude was purified by HPLC to give pure product. MS (ESI+) m/z: 862.29 [M+H]⁺. ¹H NMR (formic salt, 400 MHz, Chloroform-d) δ 8.01-7.89 (m, 2H), 7.52-7.40 (m, 1H), 7.35 (t, 2H), 4.98 (dd, 1H), 4.72 (d, 1H), 4.58 (d, 1H), 4.33 (t, 1H), 4.05-3.82 (m, 2H), 3.60 (d, 1H), 3.53-3.39 (m, 2H), 3.30 (s, 3H), 3.13 (s, 3H), 3.02-2.80 (m, 5H), 2.79-2.58 (m, 4H), 2.47 (p, 1H), 2.31 (dd, 2H), 2.19 (s, 7H), 2.01 (s, 4H), 1.92-1.75 (m, 6H), 1.75-1.27 (m, 7H), 1.27-1.13 (m, 12H), 1.13-1.03 (m, 1H), 1.00 (d, 3H), 0.87 (dd, 1H), 0.79 (d, 3H), 0.70 (d, 3H).

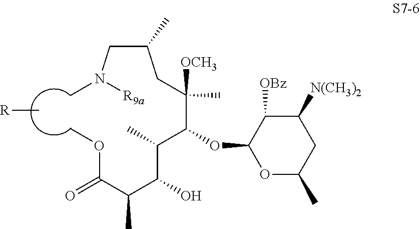

General procedure: A solution of S7-5, wherein R is cladinose, (1 eq) in DCM (0.12 M) was stirred with HCl in dioxane (4 M, 6 eq) for 20 min. Water (same volume as DCM) was added. The DCM layer was separated, and the water layer was extracted with DCM (1 times). The water layer was treated with saturated, aqueous NaHCO₃, and the water solution was extracted with DCM (3 times). The combined DCM extracts were washed with brine, dried over Na₂SO₄, filtered, and concentrated. The residue was dried under vacuum to give the desired compounds, S7-6.

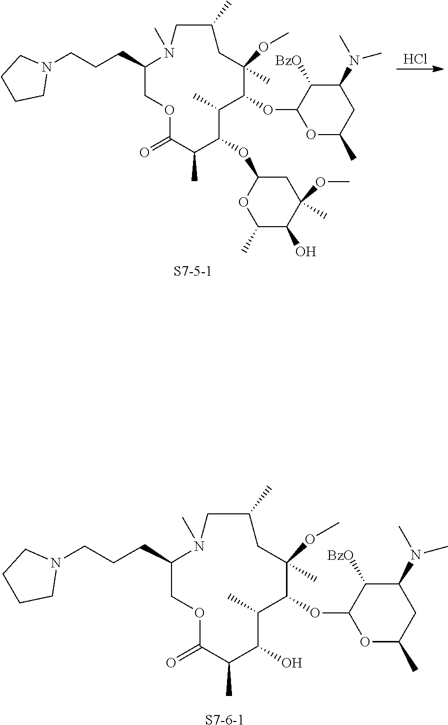

Crude compound S7-5-1 (300 mg, 0.347 mmol) in DCM (3 mL) was stirred with HCl in dioxaxe (4 M, 0.52 mL) for 25 min. The reaction mixture turned dark blue. After diluting with water (10 mL), the water layer was washed with DCM (2×10 mL). Then water layer was treated with sat. NaHCO₃ slowly, until bubbling ceased (pH 8-9). This water solution was extracted with DCM (3×10 mL). The combined extracts were dried over Na₂SO₄, were filtered, and were concentrated under vacuum to give compound S7-6-1 as a white foam, 223 mg (91%). MS (ESI+) m/z: 704.32 [M+H]⁺. ¹H NMR (400 MHz, Chloroform-d) δ 8.14-8.06 (m, 2H), 7.53 (t, 1H), 7.45 (t, 2H), 5.06-4.85 (m, 2H), 4.06 (t, 1H), 3.78 (d, 1H), 3.68-3.52 (m, 2H), 3.39 (d, 1H), 3.11 (s, 3H), 2.83-2.68 (m, 2H), 2.56-2.40 (m, 7H), 2.33 (s, 3H), 2.30-2.28 (m, 1H), 2.23 (s, 6H), 1.86-1.74 (m, 6H), 1.71 (d, 1H), 1.61-1.43 (m, 5H), 1.35-1.24 (m, 9H), 1.17-1.06 (m, 1H), 0.89 (d, 6H), 0.38 (d, 3H).

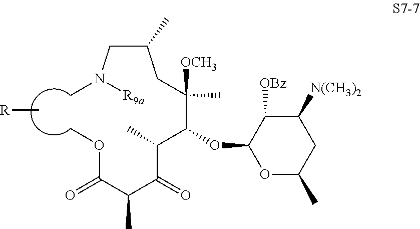

General procedure: A solution of N-chlorosuccinimide (2 eq) in anhydrous DCM (0.2 M) was stirred at −15 to −20° C. for 10 min. Dimethyl sulfide (2 eq) was added to the solution. After stirring for 20 min, a solution of S7-6 (1 eq) in DCM (0.2 M) was added dropwise to the suspension. The resulting mixture was stirred at −15 to −20° C. for 30 min, at which point triethylamine (2.5 eq) was added. The mixture was stirred at −10° C. for 40 min. Saturated aqueous NaHCO₃ solution was added. The organic layer was separated, washed with water and brine, dried over Na₂SO₄, filtered, and concentrated. The residue was purified by silica gel chromatography.

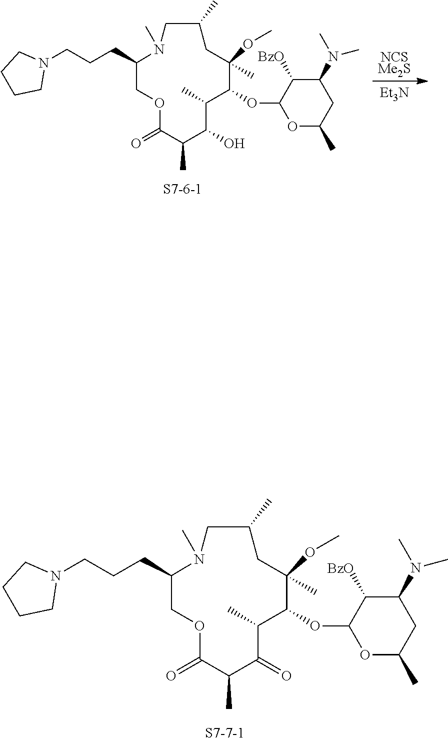

A solution of N-chlorosuccinimide (300 mg, 0.226 mmol) in anhydrous DCM (1 mL) was stirred at −15 to −20° C. for 10 min. Dimethyl sulfide (16.5 μL in 0.2 mL of DCM) was then added. After stirring for 20 min. at the same temperature, a DCM solution (0.5 mL) of S7-6-1 (80 mg, 0.113 mmol) was added dropwise to the suspension over 5 min, and the resulting mixture was stirred at −15 to −20° C. for another 30 min. TEA (39.2 μL) was added to the suspension and the resulting mixture was stirred at −10° C. for 30 min. The reaction was quenched by adding saturated aqueous NaHCO₃ solution, the organic layer was separated, washed again with sat. NaHCO₃/H₂O. The organic layer was dried over anhydrous Na₂SO₄, filtered and concentrated. Residue was purified by ISCO with 20% MeOH in DCM with 0.5% NH₄OH to give compound S7-7-1 as white foam, 52 mg (65.5%). MS (ESI+) m/z: 702.3 [M+H]⁺. ¹H NMR (400 MHz, CDCl₃) δ 8.10-7.93 (m, 2H), 7.58-7.47 (m, 1H), 7.43 (t, 2H), 5.03 (dd, 1H), 4.54 (d, 1H), 4.41-4.18 (m, 1H), 4.06 (d, 1H), 3.65-3.50 (m, 3H), 3.34-3.15 (m, 1H), 2.86-2.69 (m, 4H), 2.58 (t, 4H), 2.53-2.37 (m, 3H), 2.25 (d, 6H), 2.17 (d, 3H), 1.98-1.68 (m, 7H), 1.53 (ttd, 4H), 1.42 (td, 1H), 1.26 (d, 4H), 1.21 (d, 3H), 1.18 (d, 3H), 1.15-1.08 (m, 1H), 0.98 (p, 4H), 0.83 (d, 3H).

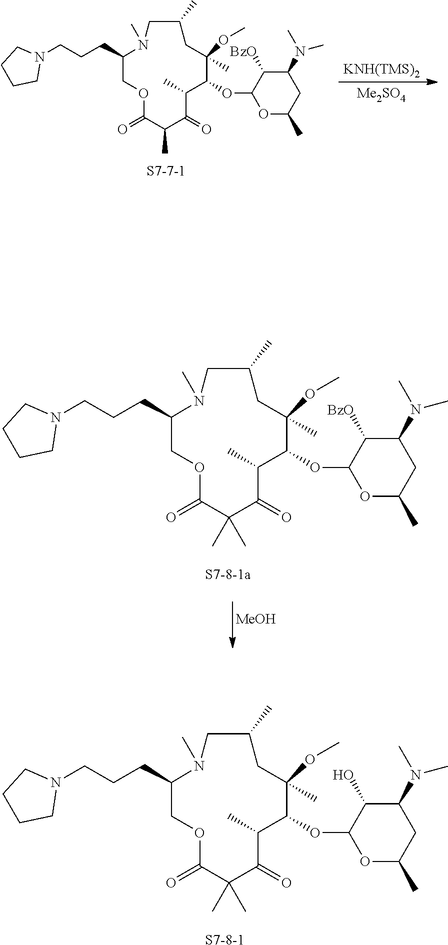

Compound S7-7-1 (370 mg, 0.527 mmol) was dissolved in dry THF (2.65 L) and DME (2.65 mL), then evacuated and filled with N₂, then the solution was cooled to −72° C. (internal) in a dry ice/Acetone bath. After stirring for 15 min KHMDS (0.685 mL, 1M, 0.685 mmol) was added drop-wise (temperature ≤−68° C.), and the mixture was stirred for 10 min at −72° C. Dimethyl sulfate (75 μL, 0.79 mmol) was added, and the reaction mixture was allowed to warm up slowly and was stirred at −15° C. in 1.5 h. The reaction mixture was cooled to −40° C., was quenched with Me₃N (1.21 mL, 40%4 in water, 7.9 mmol) and NH₄OAc (sat, aq, 10 mL), was warmed up to 10° C., and was stirred for 5 min. The mixture was diluted with EtOAc (20 mL), and the organic layer was separated and washed with water (2×10 mL) and brine (10 mL). After drying over Na₂SO₄, the solvent was removed and the residue was purified by ISCO (A: DCM, B: 20% MeOH in DCM with 0.5% NIH40H), product S7-8-1a was out at 100%/B as a white solid, 270 mg, 72%. (ESI+) m/z: 716.36 [M+H]⁺. ¹H NMR (400 MHz, CDCl₃) δ 8.10-7.94 (m, 2H), 7.62-7.48 (m, 1H), 7.43 (t, 2H), 5.03 (dd, 1H), 4.61 (d, 1H), 4.05 (dd, 1H), 4.01-3.88 (m, 2H), 3.63-3.54 (m, 1H), 3.47 (dq, 1H), 2.95-2.86 (m, 1H), 2.84 (s, 3H), 2.82-2.76 (m, 1H), 2.48 (t, 5H), 2.41 (t, 2H), 2.25 (s, 6H), 2.22 (s, 2H), 2.00-1.84 (m, 2H), 1.76 (dq, 7H), 1.56-1.44 (m, 3H), 1.38 (s, 3H), 1.31 (s, 3H), 1.26 (d, 3H), 1.22 (s, 3H), 1.04 (d, 3H), 0.98-0.86 (m, 2H), 0.82 (d, 3H).

Compound S7-8-1a (200 mg, 0.28 mmol) in MeOH (5 mL) was heated at 50° C. for 16 h. HPLC purification gave S7-8-1 in a solution of 0.1% formic acid/water/CH₃CN. The solution was concentrated and treated with sat NaHCO₃. This was extracted with DCM (3×10 mL), and the combined extracts were washed with brine (10 mL) and were dried over Na₂SO₄. After filtration, the solvent was removed under vacuum, and the residue was dried under vacuum to give S7-8-1 as a white foam, 120 mg, 70%. (ESI+) m/z: 612.37 [M+H]⁺. ¹H NMR (400 MHz, Chloroform-d) δ 4.45 (d, 1H), 4.26 (t, 2H), 3.85-3.63 (m, 2H), 3.41 (ddd, 3H), 3.31-3.21 (m, 5H), 3.15 (t, 2H), 3.05 (s, 5H), 2.78 (s, 9H), 2.19 (s, 1H), 2.13-1.97 (m, 6H), 1.86 (d, 3H), 1.60 (s, 3H), 1.54 (s, 4H), 1.45-1.23 (m, 12H), 1.05 (d, 3H).

### Alternative Azalide Synthesis from S5-6 (Scheme 5)

An alternative azalide synthesis is depicted in Scheme 8. Coupling of S5-6 with S7-1 under reductive amination conditions provides S8-1. N-substitution at the N-9a position under reductive amination conditions using an aldehyde R₅—CHO or similar ketone provides S8-2 wherein R₉ₐ is R₅—CH₂—. Conversion of S8-2 to the acid S8-3, followed by formation of the mixed anhydride and cyclization provides S8-4. Removal of the C-5 cladinose of S8-4 provides S8-5. Oxidation, C-2 alkylation wherein R_{2b} is as defined for compounds of formula I and deprotection of the C-5 desosamine completes the process to provide azalide S8-6.

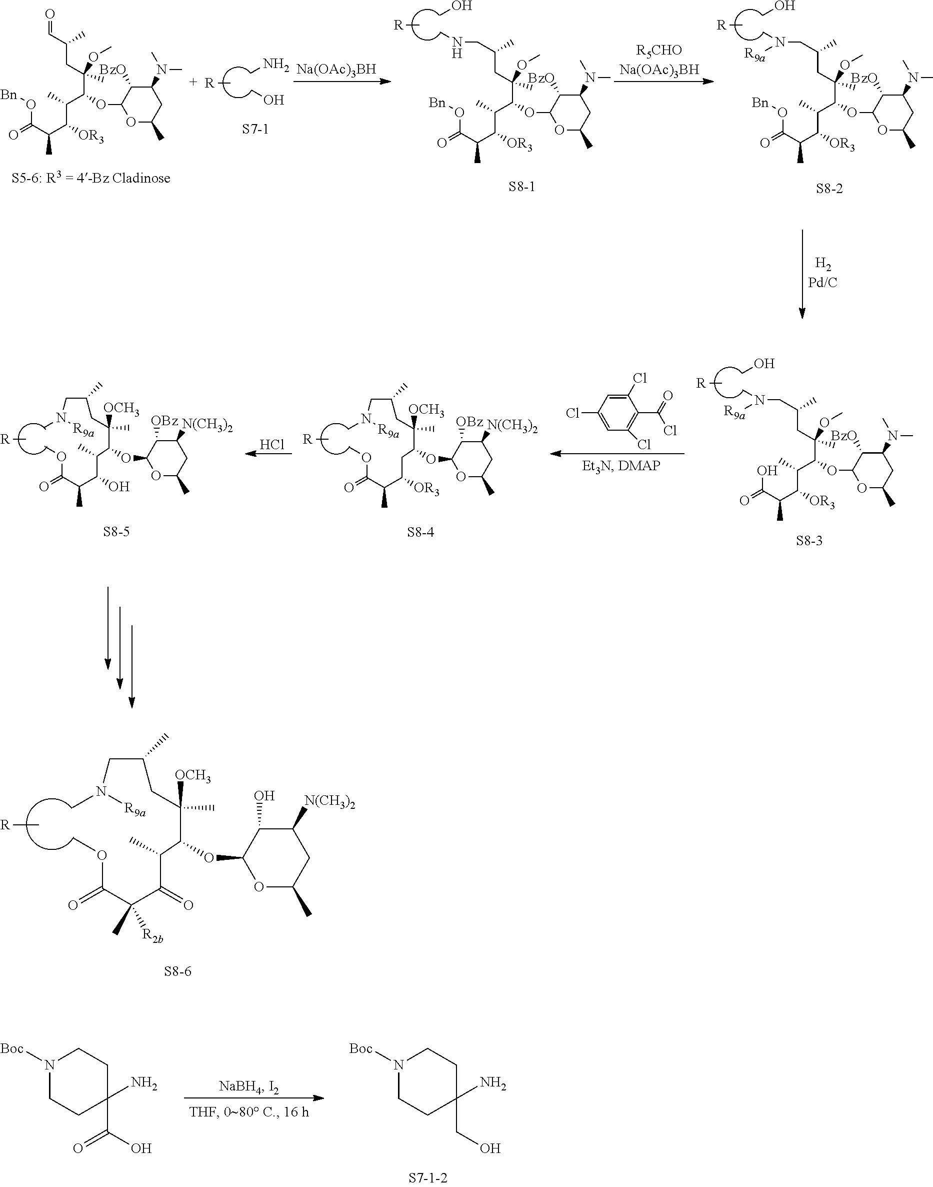

To a solution of NaBH₄ (19.7 g, 521 mmol) in THF (200 mL) was added 4-amino-1-tert-butoxycarbonyl-piperidine-4-carboxylic acid (53.00 g, 216 mmol) in one portion at 20-25° C. The reaction mixture was cooled to 0° C. under N₂, then a solution of 12 (55 g, 216 mmol) in THF (500 mL) was added slowly. Then the mixture was stirred at 80° C. for 16 h. TLC (dichloromethane:methanol=10:1, R_{f}=0.13) showed the starting material was consumed completely and one new spot formed. The reaction mixture was cooled to 25° C. and MeOH (500 mL) was added cautiously until the mixture became clear. After stirring for 1 h at RT, the solvent was removed to afford a white residue, which was taken up in 20% aq. KOH (500 mL) and stirred at 20-30° C. for 1 h. The reaction was extracted with CH₂Cl₂ (300 mL, 3×) and the combined organic extracts were dried over Na₂SO₄, filtered and concentrated. The crude product was purified by silica gel column chromatography (Dichloromethane/Methanol=40˜30/1, 0.5% NH₃H₂O). The product S7-1-2 tert-butyl 4-amino-4-(hydroxymethyl)-piperidine-1-carboxylate (R_{f}=0.30, Dichloromethane/Methanol=10/1, 0.5% NH₃H₂O) was obtained (33.5 g, 144 mmol, 66.7% yield, 99.5% purity as a white solid. MS (ESI+) m/z: 461.4 [2M+H]⁺. ¹H NMR (400 MHz, CHLOROFORM-d) δ 3.72-3.56 (m, 2H), 3.36 (s, 2H), 3.28 (ddd, 2H), 1.68 (s, 2H), 1.59-1.49 (m, 2H), 1.46 (s, 9H), 1.43-1.36 (m, 2H).

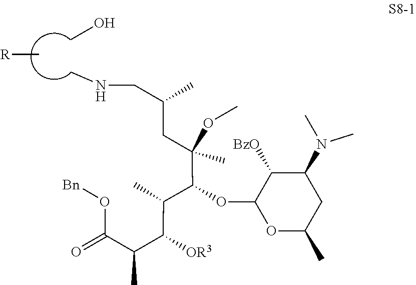

General procedure: S7-1 (1.2 eq) in DCM (0.2 M) was stirred with acetic acid (2 eq) and Na(OAc)₃BH (2 eq) at 0° C. for 10 min. S5-6 (1 eq) was added. The reaction mixture was stirred at rt until complete (30 min to overnight). The solution was used directly in next step.

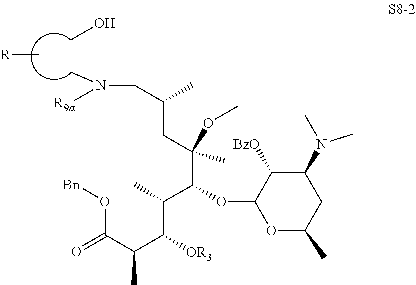

General procedure: To the above solution was added R₅CHO (3 eq) and Na(OAc)₃BH (2 eq). The resulting mixture was stirred for 30 min to overnight. The mixture was diluted with EtOAc (3×DCM volume) and was washed with saturated, aqueous NaHCO₃ and brine. The organic layer was separated, dried over Na₂SO₄, filtered, and concentrated. The residue was purified by ISCO (gradient of A: DCM, B: 20% MeOH in DCM with 0.5% NH₄OH) to give the desired compounds, S8-2.

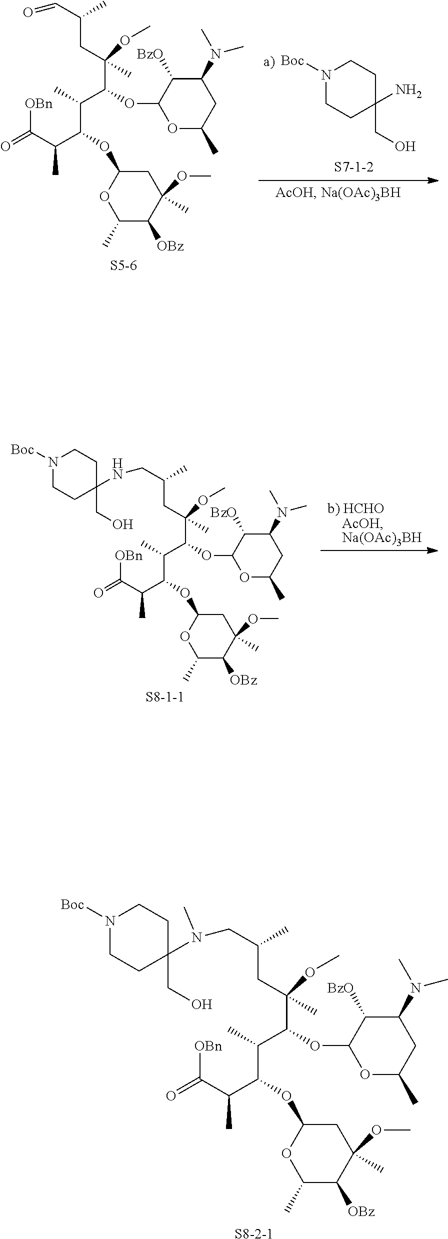

To a solution of compound S7-1-2 (23 g, 100 mmol) in CH₂Cl₂ (600 mL) was added AcOH (20 g, 333 mmol, 19 mL) and NaBH(OAc)₃ (35.3 g, 166 mmol) at 25° C. After the solution was cooled to −10° C., S5-6 (75.4 g, 83.4 mmol) in CH₂Cl₂ (150 mL) was added slowly under N₂. The reaction solution was warmed up slowly and stirred at 20-25° C. for 2 h. S8-1-1 was seen in the solution as major peak in LCMS, MS (ESI) m/z: 1118.6[M+H]⁺. Cooled to 5-10° C., then NaBH(OAc)₃ (35.3 g, 166 mmol) and HCHO (135 g, 1670 mmol, 124 mL, 37% in water) were added to the mixture. The reaction mixture was warmed up slowly and stirred at 20-25° C. for 12 h. The reaction mixture was cooled to 0° C., then sat. aq. NaHCO₃ (900 mL) was added slowly. The mixture was extracted with CH₂Cl₂ (2×1.5 L). The combined extracts were dried over Na₂SO₄, filtered and concentrated to give a crude product. The crude was purified by column chromatography (SiO₂, dichloromethane:methanol=100:1 to 40:1; Plate 1; R_{f}=0.24) to give compound S8-2-1 (80 g, 63.8 mmol, 76.6% yield, 96% purity) as a white solid. MS (ESI) m/z: 1132.7[M+H]⁺, 567.0 [M/2+H]⁺. ¹H NMR (400 MHz, CHLOROFORM-d) δ7.97-8.05 (m, 4H), 7.46-7.65 (m, 4H), 7.39-7.46 (m, 2H), 7.29-7.39 (m, 5H), 5.07-5.19 (m, 2H), 4.97-5.04 (m, 1H), 4.92 (d, 1H), 4.79-4.89 (m, 2H), 4.34-4.46 (m, 1H), 3.75-4.07 (m, 6H), 3.71 (br s, 1H), 3.45-3.50 (m, 3H), 3.39 (s, 3H), 3.17 (br s, 3H), 2.79-2.99 (m, 3H), 2.65-2.79 (m, 4H), 2.62 (s, 2H), 2.25-2.58 (m, 8H), 1.86 (s, 2H), 1.55-1.70 (m, 3H), 1.44 (s, 9H), 1.20-1.40 (m, 6H), 1.05-1.19 (m, 12H), 0.95 (d, 3H), 0.64 (br d, 3H).

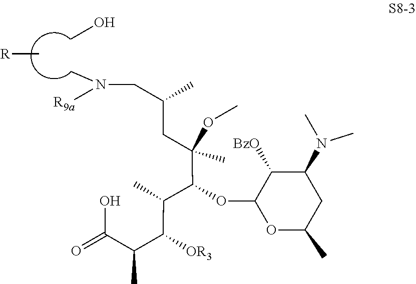

General procedure: To a solution of S8-2 in DCM (10 mM) was added 10% Pd/C (10 to 50 mol %). The reaction vessel was evacuated and back-filled with hydrogen. Hydrogen was bubbled through the solution for 2-3 h. The mixture was filtered through Celite, and washed with MeOH. The filtrate was concentrated, and the residue was dried under vacuum.

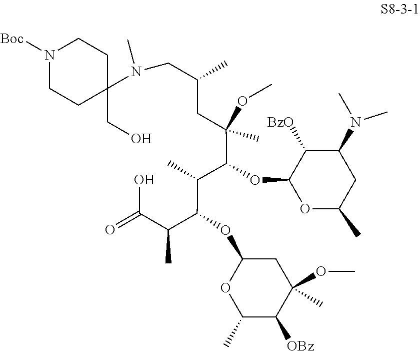

To a solution of compound S8-2-1 (50.00 g, 39.3 mmol) in MeOH (500 mL) was added 10% Pd/C (25.00 g, 50% weight of S8-2-1) under N₂ atmosphere. The mixture was stirred under H₂ (15 psi) at 25° C. for 2 h. The reaction mixture was filtered under vacuum and the filter cake was washed with MeOH (2×1000 mL). The filtrate was concentrated to give S8-3-1 as a white solid. MS (ESI) m/z: 522.0 [M/2+H]⁺. ¹H NMR (400 MHz, CHLOROFORM-d) δ 8.10-7.96 (m, 4H), 7.64-7.51 (m, 2H), 7.50-7.38 (m, 4H), 5.17-4.87 (m, 4H), 4.59-4.43 (m, 1H), 4.10-3.64 (m, 7H), 3.52 (s, 3H), 3.31 (br s, 3H), 3.07-2.76 (m, 3H), 2.71-2.42 (m, 6H), 2.33 (s, 7H), 2.15 (br d, 1H), 2.00-1.85 (m, 1H), 1.82-1.56 (m, 6H), 1.51-1.40 (m, 10H), 1.35-1.26 (m, 3H), 1.24-1.08 (m, 9H), 1.04-0.93 (m, 6H), 0.73 (br d, 3H).

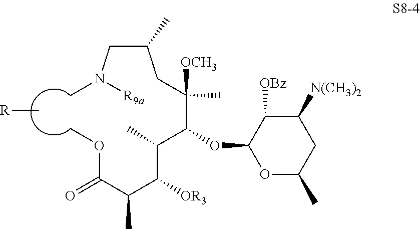

General procedure: To a solution of S9-3 (1 eq) in dry DCM (38 V) was added Et₃N (2 eq) and 1,3,5-trichlorobenzoyl chloride (1.2 eq). After 30 min, DMAP (0.02 eq) was added. The mixture was stirred at rt for 30 min. The solution was washed with saturated, aqueous NaHCO₃ (3 times) and brine (1 time). The DCM solution was dried over Na₂SO₄, filtered, and concentrated. The residue was dried under vacuum to give the desired compounds, S8-4.

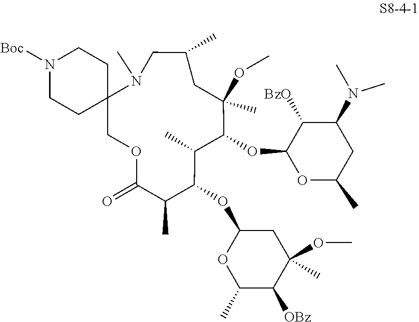

To a solution of 2,4,6-trichlorobenzoyl chloride (16.2 g, 66.6 mmol, 10.4 mL) in CH₂Cl₂ (3900 mL) in a 5 L three-necked flask were added Et₃N (17.9 g, 177 mmol, 24.7 mL) and DMAP (5.43 g, 44.4 mmol) at 20-25° C. Then compound S8-3-1 (26 g, 22.2 mmol) in CH₂Cl₂ (260 mL) was added slowly to the mixture by a syringe pump over 48 h. After addition, the reaction was stirred at 20-25° C. for 2 h. The reaction solution was washed with saturated NaHCO₃ (3×600 mL). The solvent was dried over Na₂SO₄ and removed under vacuum, the residue was purified by column chromatography (SiO₂, dichloromethane:methanol=100:1 to 30:1; Plate 1; R₁=0.24) to give compound S8-4-1 (2 batches, 31.9 g, 27 mmol, 50.6% yield, 86.7% purity) as a white solid. MS (ESI) m/z: 512.9 [M/2+H]⁺. ¹H NMR (400 MHz, CHLOROFORM-d) δ 8.02-8.13 (m, 4H), 7.51-7.63 (m, 2H), 7.41-7.51 (m, 4H), 5.03-5.15 (m, 1H), 4.89-4.99 (m, 2H), 4.49-4.59 (m, 2H), 4.12-4.21 (m, 1H), 3.68-3.85 (m, 4H), 3.45 (s, 3H), 3.25 (s, 3H), 2.82-2.99 (m, 3H), 2.57-2.69 (m, 1H), 2.46 (d, 1H), 2.26-2.38 (m, 8H), 2.11-2.26 (m, 5H), 1.61-1.83 (m, 7H), 1.46 (s, 10H), 1.15-1.31 (m, 14H), 1.05 (d, 3H), 0.78-0.92 (m, 7H).

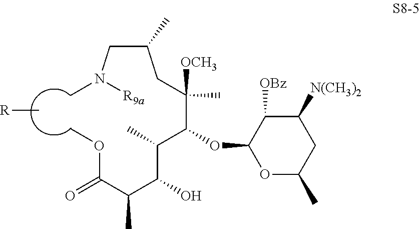

General procedure: A solution of S8-4 (1 eq) in DCM (0.12 M) was stirred with HCl in dioxane (4 M, 6 eq) for 20 min. Water was added, the DCM layer was separated, and the water layer was extracted with DCM (1 times). The water layer was treated with saturated, aqueous NaHCO₃ and was extracted with DCM (3 times). The combined DCM extracts were washed with brine, dried over Na₂SO₄, filtered, and concentrated. The residue was dried under vacuum to give the desired products, S8-5.

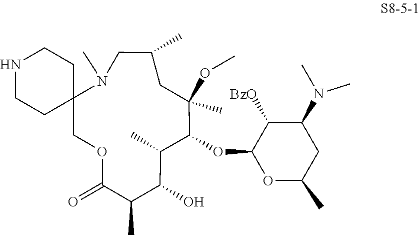

To a solution of compound S8-4-1 (31.9 g, 27 mmol) in CH₂Cl₂ (320 mL) was added trifluoroacetic acid (92.3 g, 810 mmol, 59.9 mL) at 0-5° C. The mixture was stirred at 20-25° C. for 3 h. Water (300 mL) was added to the reaction and the water layer was separated. After extracting with CH₂Cl₂ (2×80 mL), the water layer was adjusted to pH 8-9 with Na₂CO₃ solid. The water was extracted with CH₂Cl₂ (4×100 mL). The combined extracts were dried over Na₂SO₄, filtered and concentrated under vacuum. The residue was purified by column chromatography (SiO₂, dichloromethane:methanol=60:1 to 10:1; Plate 1; R_{f}=0.24) to give compound S8-5-1 (21 g, 26.4 mmol. 98.1% yield) as a white solid. MS (ESI) m/z: 662.4 [M+H]⁺. ¹H NMR (400 MHz, METHANOL-d4) δ 8.14-7.97 (m, 3H), 7.59-7.48 (m, 1H), 7.48-7.42 (m, 1H), 7.48-7.42 (m, 1H), 7.50-7.38 (m, 2H), 7.37-7.34 (m, 1H), 5.11-4.90 (m, 3H), 4.11 (s, 2H), 3.82 (d, 1H), 3.61 (br dd, 2H), 3.44 (br d, 1H), 3.21-3.07 (m, 5H), 3.06-2.93 (m, 3H), 3.07-2.88 (m, 4H), 2.86-2.55 (m, 5H), 2.46-2.33 (m, 3H), 2.24-2.23 (m, 1H), 2.30-2.14 (m, 15H), 1.88-1.75 (m, 4H), 1.70-1.54 (m, 4H), 1.38-1.13 (m, 13H), 0.98-0.78 (m, 8H), 0.49 (d, 3H).

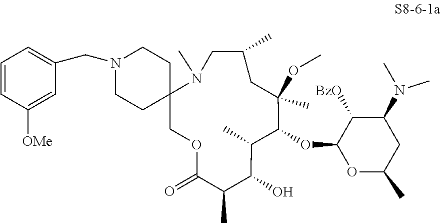

To a solution of 3-methoxybenzaldehyde (5.41 g, 39.7 mmol) in CH₂Cl₂ (100 mL) was added AcOH (6.36 g, 105 mmol, 6.06 mL) and NaBH(OAc)₃ (11.2 g, 52.9 mmol) at 0° C. Then compound S8-5-1 (21.0 g, 26.4 mmol) in CH₂Cl₂ (100 mL) was added slowly at 0-10° C. The mixture was warmed up to 20-25° C. and stirred for 6 h. To the reaction mixture was added sat. aq. NaHCO₃ (400 mL) and CH₂Cl₂ (100 mL) at 0° C., and the mixture was extracted with CH₂Cl₂ (2×100 mL). The combined extracts were washed with brine (200 mL), dried over Na₂SO₄, filtered and concentrated to give a residue. The residue was purified with MEGA Series LC Pump in 0.50 Kg of column, cartridge (SiO₂: 0.50 Kg), A: DCM with 0.1% NH₄OH, B. MeOH), most of product was out at ˜2% B, some product out until 4% B. Compound S8-6-1a (11.8 g, 14.9 mmol, 56.3% yield, 98.9% purity) was obtained as a brown foam. MS (ESI) m/z: 782.6 [M+H]⁺. ¹H NMR (400 MHz, METHANOL-d4) δ 8.01-8.17 (m, 2H), 7.50-7.60 (m, 1H), 7.36-7.49 (m, 2H), 7.23 (t, 1H), 6.89 (br d, 2H), 6.80 (dd, 1H), 4.98 (br d, 2H), 4.06 (s, 2H), 3.78-3.86 (m, 4H), 3.55-3.65 (m, 1H), 3.40-3.52 (m, 3H), 3.16 (s, 3H), 2.71-2.86 (m, 3H), 2.64 (br s, 1H), 2.36 (br dd, 2H), 2.24 (s, 10H), 1.97-2.15 (m, 2H), 1.62-1.90 (m, 7H), 1.40-1.59 (m, 4H), 1.32 (t, 6H), 1.27 (br d, 2H), 0.90 (dd, 6H), 0.50 (d, 3H).

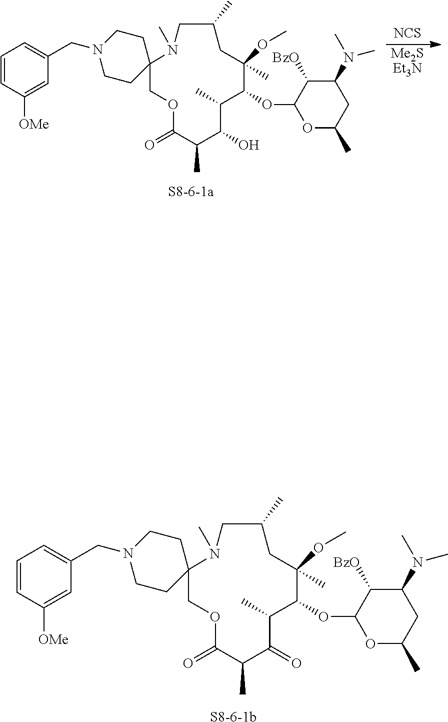

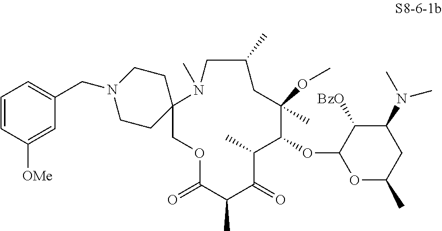

A mixture of NCS (13 g, 97 mmol) in CH₂Cl₂ (118 mL) was stirred at −15 to −20° C. for 10 min under N₂. Me₂S (6.03 g, 97 mmol, 7.12 mL) was added dropwise to the solution. The mixture was stirred at the same temperature for 30 minutes. A mixture of compound S8-6-1a (11.8 g, 14.9 mmol) in CH₂Cl₂ (118 mL) was added dropwise to the suspension at −15 to −20° C., then the resulting mixture was stirred at −15 to −10° C. for another 1 h. TEA (10.5 g, 104 mmol, 14.5 mL) was added into the mixture at −15 to −10° C. and stirred for 1 h. The reaction was quenched by adding saturated aqueous NaHCO₃ solution (224 mL) at 0-10° C. The organic layer was separated. The water layer was extracted with CH₂Cl₂ (2×160 mL). The combined extracts were dried over anhydrous Na₂SO₄, filtered, and concentrated. The residue was purified with MEGA Series LC Pump in 0.25 Kg of column, cartridge (SiO₂: 0.25 Kg), A: DCM with 0.2% NH₄OH, B: MeOH), most of product was out at ˜2% B, some product out until 4% B. Compound 8-6-1b (8.55 g, 10.4 mmol, 69.7% yield, 94.9% purity) was obtained as a brown foam. MS (ESI) m/z: 780.4[M+H]⁺, 390.9 [M/2+H]⁺. ¹H NMR (400 MHz, METHANOL-d4) δ 8.04 (br d, 2H), 7.50-7.59 (m, 1H), 7.38-7.49 (m, 2H), 7.22 (t, 1H), 6.89 (br d, 2H), 6.80 (br d, 1H), 4.99-5.14 (m, 1H), 4.46-4.64 (m, 1H), 4.10-4.29 (m, 2H), 3.96-4.09 (m, 1H), 3.81 (s, 3H), 3.40-3.66 (m, 4H), 3.10-3.26 (m, 1H), 3.03 (s, 1H), 2.80-2.92 (m, 3H), 2.67-2.78 (m, 1H), 2.44-2.63 (m, 2H), 2.37 (s, 2H), 2.18-2.31 (m, 8H), 1.89-2.17 (m, 4H), 1.54-1.85 (m, 6H), 1.38-1.53 (m, 3H), 1.11-1.36 (m, 10H), 0.92-1.06 (m, 4H), 0.81-0.89 (m, 3H).

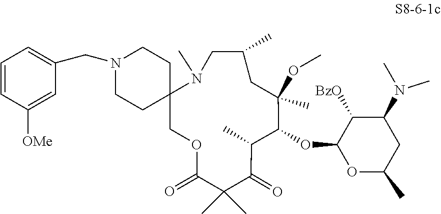

A solution of compound S8-6-1b (7.0 g, 8.52 mmol) in 2-MeTHF (70 mL) was degassed and purged with N₂ for 3 times, then the mixture was cooled to −42° C. KHMDS (1.0 M, 11 mL, 11 mmol) was added drop-wise and kept at −40 to −42° C. The mixture was stirred for 30 min at the same temperature. Me₂SO₄ (1.84 g, 14.5 mmol, 1.38 mL) was added, and the reaction mixture was allowed to warm slowly to −20 to −10° C. for 1 h. The reaction was quenched by adding saturated NH₄Cl (60 mL) and trimethylamine (120 mL, 40% in water). The reaction mixture was poured into saturated NH₄Cl (200 mL), then extracted with 2-MeTHF (2×200 mL). The combined extracts were washed with brine (200 mL), dried over Na₂SO₄, filtered and concentrated. The resulting residue was purified with MEGA Series LC Pump in 0.1 Kg of column, cartridge (SiO₂: 0.1 Kg), A: DCM with 0.1% NH₄OH, B: MeOH), most of product was out at −1% B, some product out until 4% B. The product was detected by TLC (Dichloromethane/Methanol=10/1, 0.1% NH₄OH, Rf=0.30). product S8-6-1c (6.55 g, 8.18 mmol, 78.7% yield, 99.2% purity) was obtained as a brown foam. MS (ESI) m/z: 397.9 [M/2+H]⁺. ¹H NMR (400 MHz, METHANOL-d4) δ 7.95-8.09 (m, 2H), 7.51-7.59 (m, 1H), 7.38-7.47 (m, 2H), 7.22 (t, 1H), 6.89 (br d, 2H), 6.79 (dd, 1H), 5.04 (dd, 1H), 4.62 (d, 1H), 4.07-4.21 (m, 1H), 3.92-4.05 (m, 2H), 3.81 (s, 3H), 3.54-3.68 (m, 1H), 3.36-3.53 (m, 2H), 3.13-3.28 (m, 1H), 2.93 (s, 3H), 2.77-2.88 (m, 1H), 2.65 (br d, 2H), 2.23-2.39 (m, 9H), 2.07-2.23 (m, 4H), 1.57-1.95 (m, 8H), 1.14-1.52 (m, 14H), 1.02 (d, 3H), 0.94 (br dd, 1H), 0.83 (d, 3H).

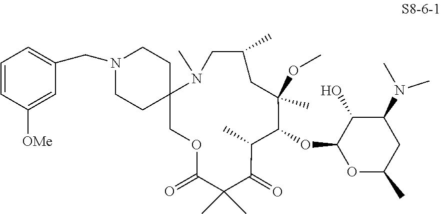 [SMILES: COc1cccc(CN2CCC3(CC2)COC(=O)C(C)(C)C(=O)[C@H](C)[C@@H](O[C@@H]2O[C@H](C)C[C@H](N(C)C)[C@H]2O)[C@](C)(OC)C[C@@H](C)CN3C)c1]

A solution of compound S8-6-1c (6.55 g, 8.18 mmol) in EtOH (66 mL) was stirred at 80° C. for 36 h. After cooling to room temperature, the reaction mixture was concentrated to give a residue. The residue was purified by prep-HPLC (column: YMC Triart C18 250*50 mm*7 um; mobile phase: [water(TFA)-ACN]; B %: 2%-27%, 20 min). Compound S8-6-1 (3.23 g, 4.66 mmol, 56.9% yield, 99.5% purity) was obtained as a light-yellow solid. (MS (ESI) m/z: 690.4 [M+H]⁺. ¹H NMR (400 MHz, METHANOL-d4) δ 7.22 (t, 1H), 6.79-6.95 (m, 3H), 5.49 (s, 1H), 4.40 (d, 1H), 4.19 (d, 1H), 3.98-4.10 (m, 2H), 3.79 (s, 3H), 3.53-3.60 (m, 1H), 3.50 (s, 2H), 3.40 (br t, 1H), 3.26 (dd, 1H), 2.95 (s, 3H), 2.54-2.76 (m, 3H), 2.09-2.48 (m, 14H), 1.59-2.06 (m, 7H), 1.46-1.58 (m, 4H), 1.37 (s, 3H), 1.15-1.33 (m, 1H), 0.85 (d, 3H).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set in verbatim herein. It is also noted that the terms “comprising” and “containing” are intended to be open and permits the inclusion of additional elements or steps.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A process for preparing a compound of formula I or formula I′: 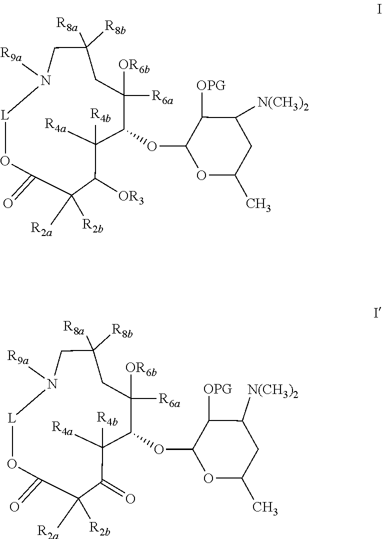
or a pharmaceutically acceptable salt thereof, comprising:
(a1) intramolecular cyclization of a compound of formula A, wherein O-LG is a leaving group and PG is a protecting group, to form a compound of formula I: 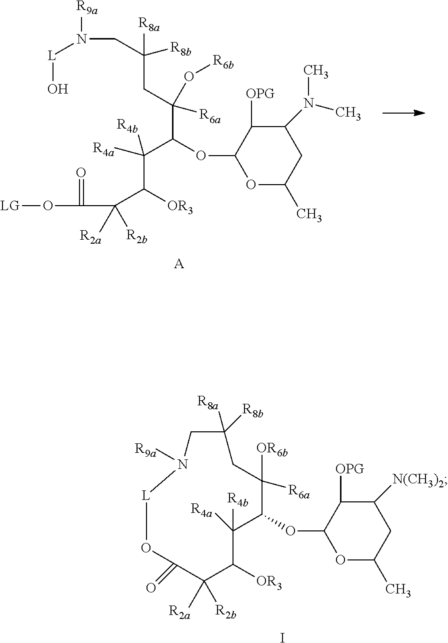
or
(b1) intramolecular cyclization of a compound of formula B, wherein PG is a protecting group, to form a compound of formula I′: 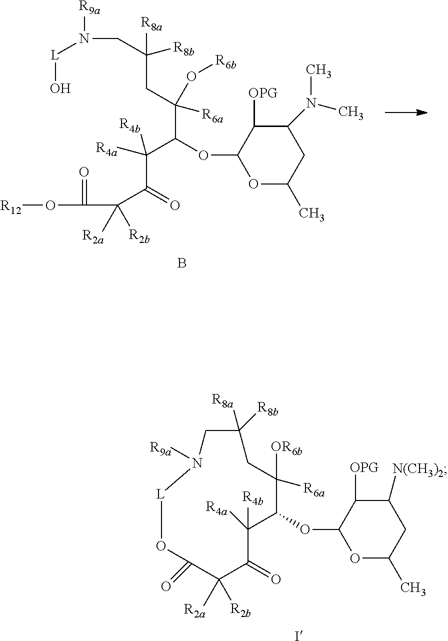
wherein:
one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is selected from the group consisting of halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
R₃ is H, a suitable protecting group selected from acyl, carbamoyl, alkyl ether, or silyl ether protecting groups, or 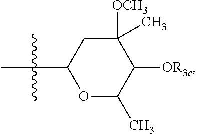
wherein R_{3c} is H or a protecting group;
each of R₄ₐ and R_{4b} is independently selected from the group consisting of —H, and optionally substituted C₁₋₁₀ alkyl;
R₆ₐ is optionally substituted C₁₋₁₀ alkyl;
R_{6b} is —H, optionally substituted C₁₋₁₀ alkyl, optionally substituted C₁₋₁₀ hydroxyalkyl, and optionally substituted allyl;
R₈ₐ and R_{8b} are each independently selected from the group consisting of —H and optionally substituted C₁₋₁₀ alkyl;
R₉ₐ is selected from the group consisting of —H and optionally substituted C₁₋₁₀ alkyl;
R₁₂ is a C₁₋₆ alkyl; and
L is a C₂₋₅ optionally substituted alkylene.

2. (canceled)

3. (canceled)

4. (canceled)

5. The process of claim 1, comprising the step of converting a compound of formula A-1 to the compound of formula A:

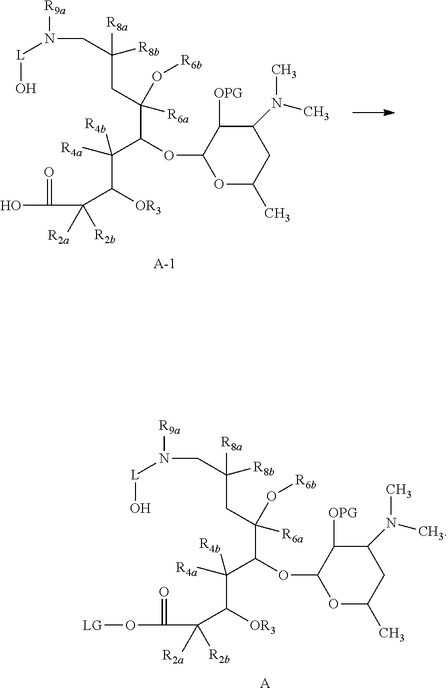

6. The process of claim 5, wherein LG is R′—(C═O)—.

7. The process of claim 6, wherein R′ is optionally substituted phenyl.

8. (canceled)

9. (canceled)

10. (canceled)

11. The process of claim 5, further comprising the step of converting a compound of formula A-2, wherein Rₓ is a C₁₋₆ alkyl, optionally substituted with C₁₋₄ alkyl, hydroxyl, oxo, COOH, COO(C₁₋₆ alkyl), amino, alkylamino, halo, or cyano, to a compound of formula A-1:

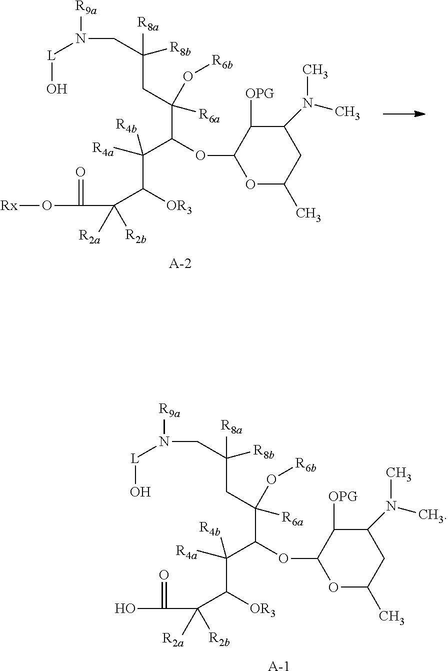

12. The process of claim 11, further comprising the step of converting the compound of formula A-3 to a compound of formula A-2:

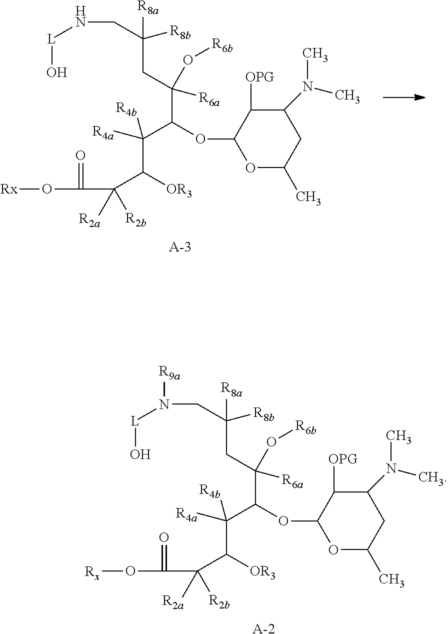

13. The process of claim 12, further comprising the step of converting the compound of formula A-4 to a compound of formula A-3

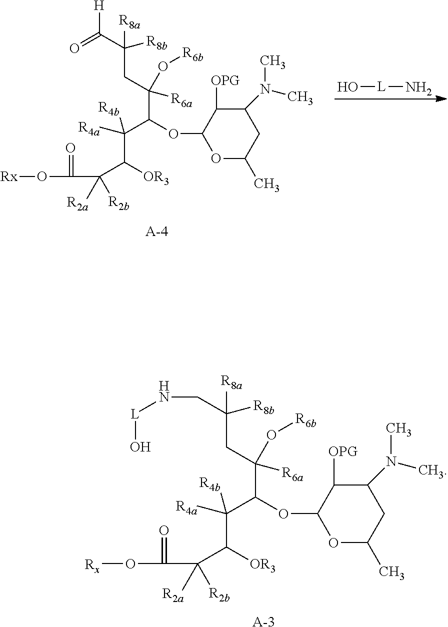

14. The process of claim 13, further comprising the step of converting the compound of formula A-4A to a compound of formula A-4, by contacting a compound of formula A-4A with an oxidizing agent:

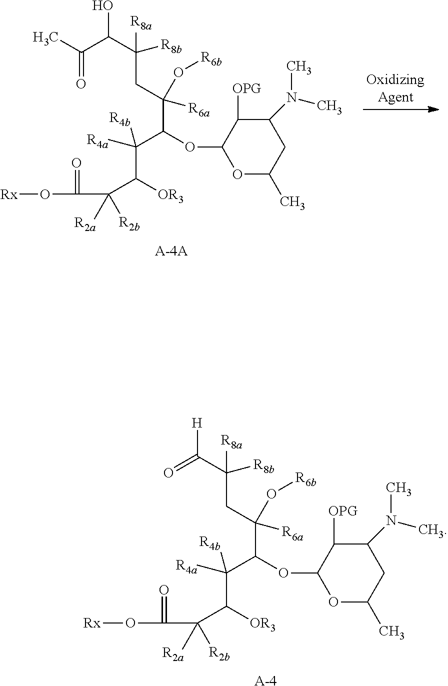

15. The process of claim 14, further comprising the step of converting the compound of formula A-4A1 to a compound of formula A-4A:

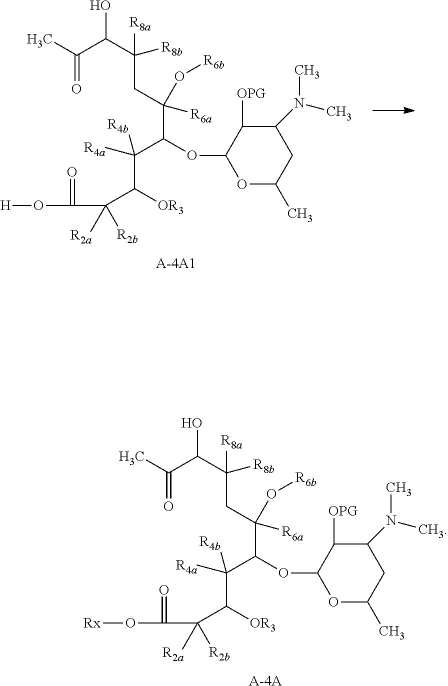

16. The process of claim 15, further comprising the step of converting the compound of formula A-4A2 to a compound of formula A-4A1:

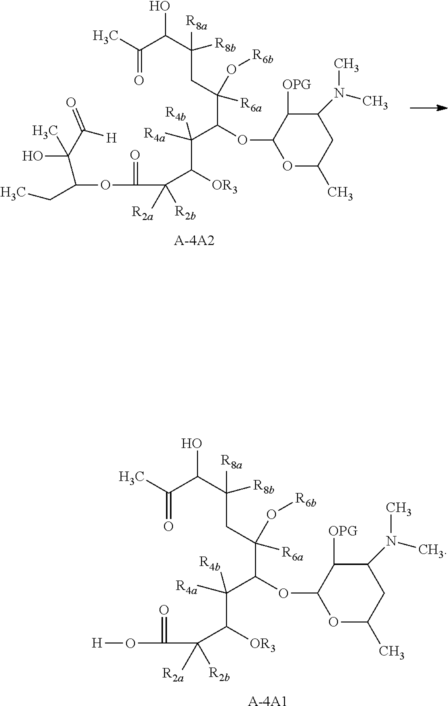

17. The process of claim 16, further comprising the step of converting the compound of formula A-4A3 to a compound of formula A-4A2:

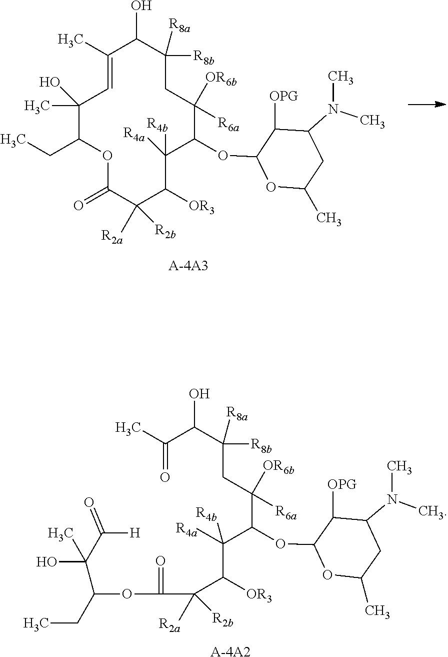

18. The process of claim 17, further comprising the step of converting the compound of formula II to a compound of formula A-4A3:

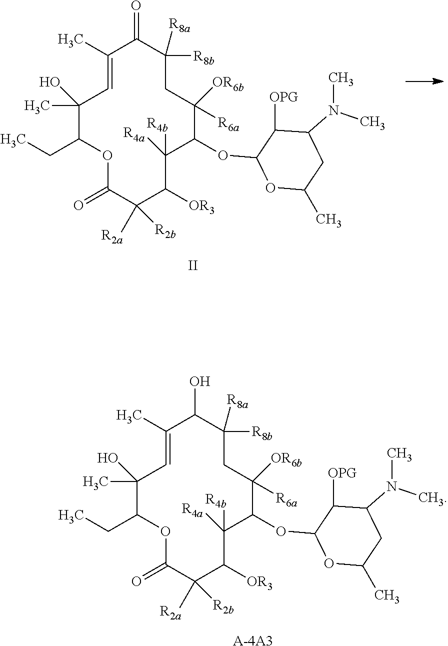

19. The process of claim 18, further comprising the step of converting the compound of formula III to a compound of formula II:

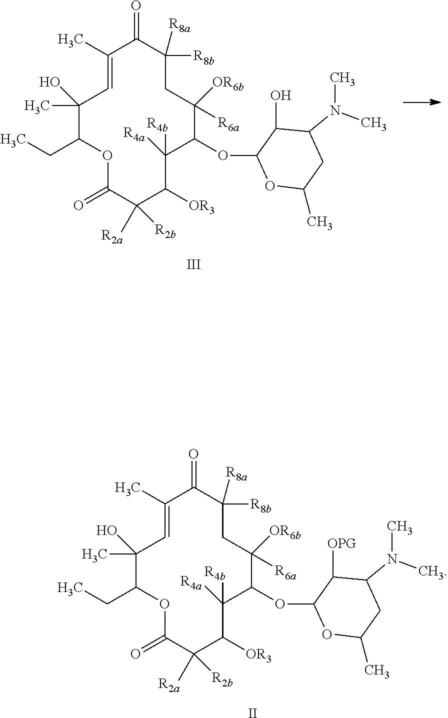

20-24. (canceled)

25. The process of claim 1, wherein R₃ is 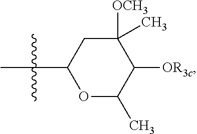
further comprising the step of converting the compound of formula I to a compound of formula IA:

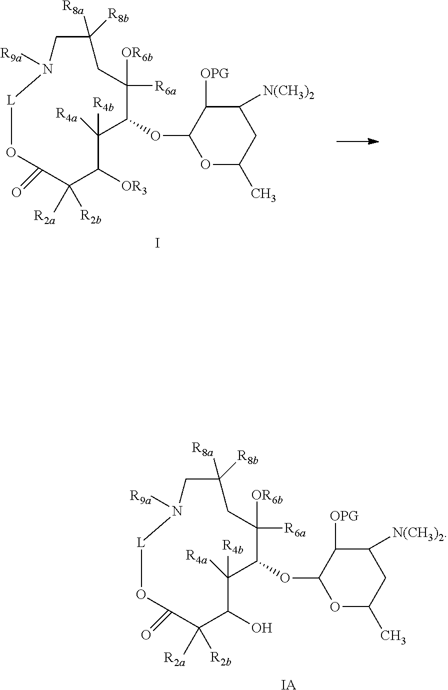

26. The process of claim 25, further comprising the step of converting the compound of formula IA to a compound of formula I′:

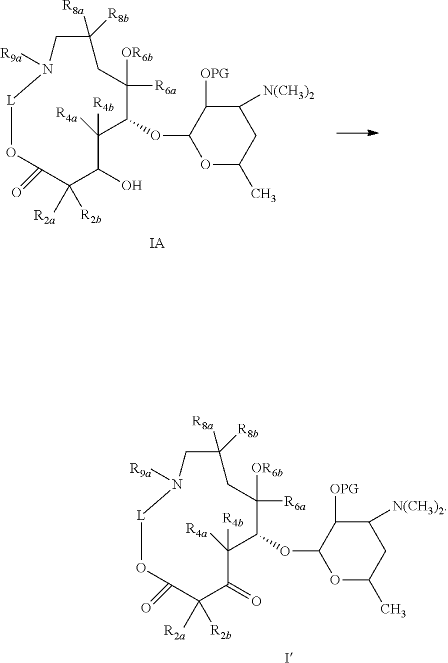

27. The process of claim 26, further comprising the step of converting the compound of formula I′ to a compound of formula IC:

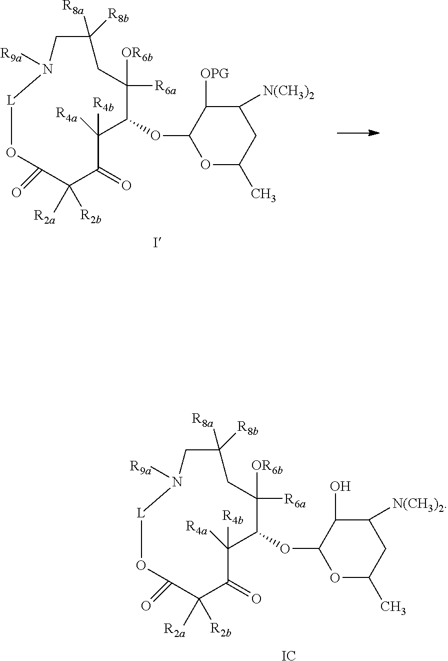

28-38. (canceled)

39. The process of claim 1, wherein one of R₈ₐ and R_{8b} is H and the other is an optionally substituted C₁₋₁₀ alkyl.

40. (canceled)

41. The process of claim 1, wherein R₆ₐ is selected from methyl, ethyl, propyl, isopropyl, and tert-butyl, and R_{6b} is selected from H, methyl, ethyl, propyl, isopropyl, and tert-butyl.

42. (canceled)

43. The process of claim 1, wherein R₃ is H, or 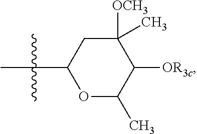
and R_{3c} is H or a benzoyl group.

44. The process of claim 1, wherein one of R₄ₐ and R_{4b} is H and the other is an optionally substituted C₁₋₁₀ alkyl.

45. (canceled)

46. The process of claim 1, wherein
one of R₂ₐ and R_{2b} is H,
and the other of R₂ₐ and R_{2b} is a C₁₋₁₀ alkyl, optionally substituted with one or more groups selected from the group consisting of halo, aryl, amino, alkyl, heteroalkyl, heteroalkenyl, heterocycloalkyl, and heteroaryl.

47-61. (canceled)

62. A process for preparing a compound of formula A-3 from a compound of formula A-4, comprising coupling the compound of formula A-4 with an amine having the formula NH₂-L-OH under reductive amination conditions to form a compound of formula A-3: 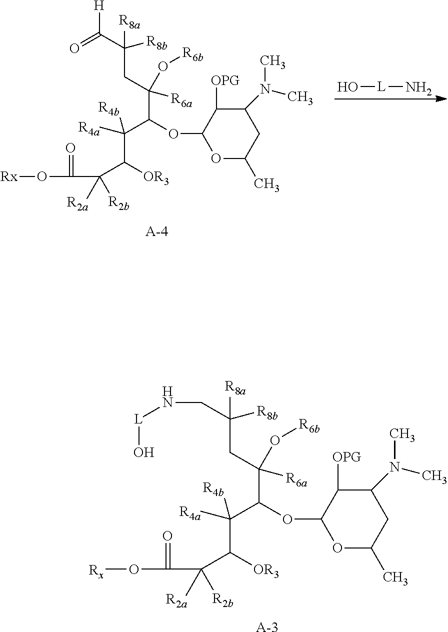
wherein:
one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
and the other of R₂ₐ and R_{2b} is selected from the group consisting of halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
R₃ is H, a suitable protecting group selected from acyl, carbamoyl, alkyl ether, or silyl ether protecting groups, or 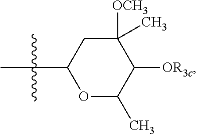
wherein R_{3c} is H or a protecting group;
each of R₄ₐ and R_{4b} is independently selected from the group consisting of —H, and optionally substituted C₁₋₁₀ alkyl;
R₆ₐ is optionally substituted C₁₋₁₀ alkyl;
R_{6b} is —H, optionally substituted C₁₋₁₀ alkyl, optionally substituted C₁₋₁₀ hydroxyalkyl, and optionally substituted allyl;
R₈ₐ and R_{8b} are each independently selected from the group consisting of —H and optionally substituted C₁₋₁₀ alkyl;
PG is a suitable protecting group; and
L is an optionally substituted C₂₋₅ alkylene.

63-66. (canceled)

67. The process of claim 62, wherein NH₂-L-OH is NH₂—CH₂-L₁-OH, NH₂—CH(C₁₋₆ alkyl)-L₁OH, NH₂-L₁-CH₂—OH or NH₂-L₁-CH(C₁₋₆ alkyl)-OH.

68. The process of claim 67, wherein L is ethylene, which is optionally substituted with up to three of halo, CN, NO₂, amino, amido, carboxy, alkylcarbonyl, alkoxycarbonyl, alkyl, alkoxy, cycloalkyl, heterocycloalkyl, aryl, or heteroaryl, wherein the alkyl, alkoxy, cycloalkyl, heterocycloalkyl, aryl, and heteroaryl are each independently and optionally substituted with halo or alkyl.

69. (canceled)

70. The process of claim 67, wherein NH₂-L-OH is NH₂-L₁-CH₂—OH.

71. The process of claim 70, wherein L₁ is CH—R₁₄ₐ, wherein R₁₄ₐ is selected from the group consisting of an optionally substituted R₁₀₁—CH₂, R₁₀₁—CH₂CH₂—, R₁₀₁—CH₂CH₂CH₂, optionally substituted R₁₀₁—CH₂CH₂CH—OH—, and optionally substituted R₁₀₁—CH₂CH₂CH—OMe-.

72. The process of claim 71, wherein R₁₀₁ is selected from the group consisting of 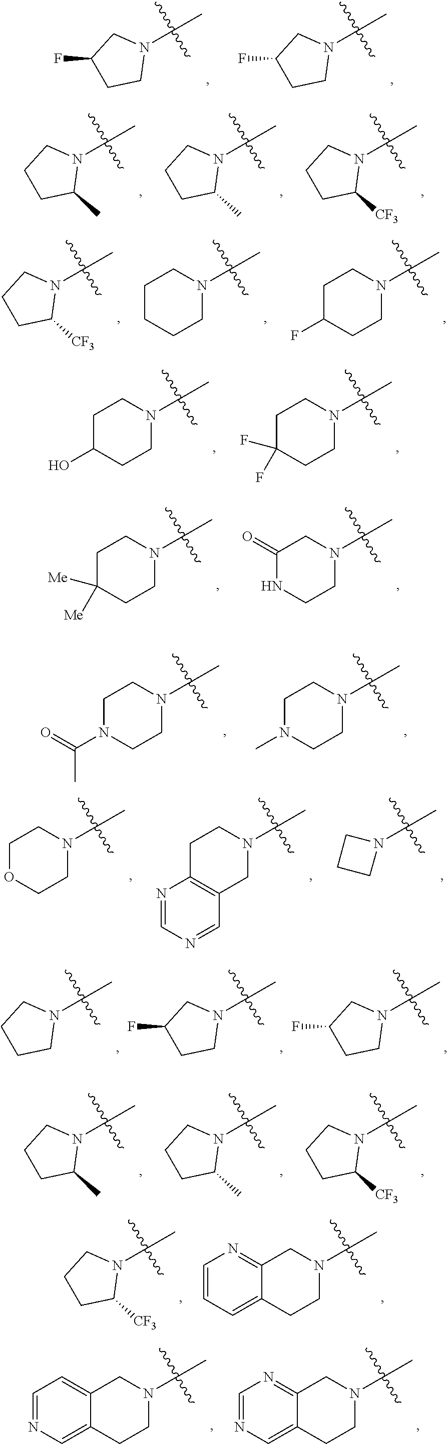 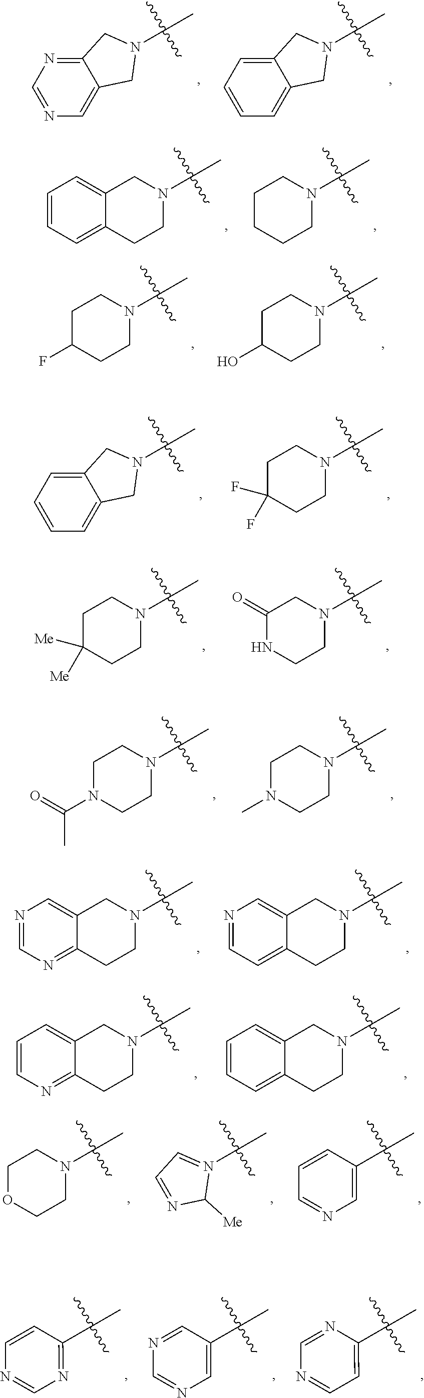 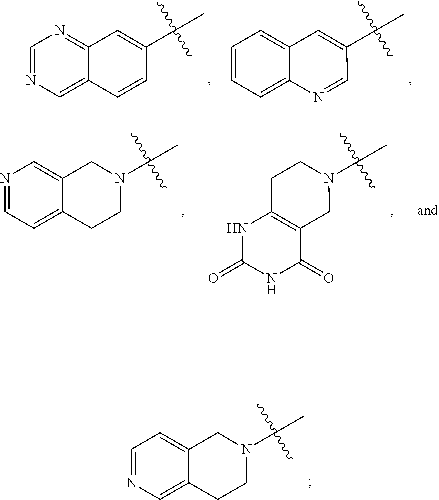 [SMILES: *c1ccc2cncnc2c1 ; *c1cnc2ccccc2c1 ; *N1CCc2ccncc2C1 ; *N1CCc2[nH]c(=O)[nH]c(=O)c2C1 ; *N1CCc2cnccc2C1]
where [symbol] indicates a point of attachment.

73. The process of claim 70, wherein L₁ is CH—R₁₄ₐ, wherein R₁₄ₐ is optionally substituted saturated or partially unsaturated cycloalkyl containing at least one double bond, optionally substituted saturated or partially unsaturated heterocycloalkyl, optionally substituted aryl, or optionally substituted heteroaryl.

74-78. (canceled)

79. The process of claim 62, wherein one of R₈ₐ and R_{8b} is H and the other is an optionally substituted C₁₋₁₀ alkyl.

80. (canceled)

81. The process of claim 62, wherein R₆ₐ is selected from methyl, ethyl, propyl, isopropyl, and tert-butyl, and R_{6b} is selected from H, methyl, ethyl, propyl, isopropyl, and tert-butyl.

82. (canceled)

83. The process of claim 62, wherein R_{3c} is a protecting group selected from benzoyl, p-nitrobenzoyl, TMS, TES IPDMS, TBS, or methoxymethyl.

84. (canceled)

85. The process of claim 62, wherein one of R₄ₐ and R_{4b} is H and the other is an optionally substituted C₁₋₁₀ alkyl.

86. (canceled)

87. The process of claim 62, wherein
one of R₂ₐ and R_{2b} is H,
and the other of R₂ₐ and R_{2b} is a C₁₋₁₀ alkyl, optionally substituted with one or more groups selected from the group consisting of halo, aryl, amino, alkyl, heteroalkyl, heteroalkenyl, heterocycloalkyl, and heteroaryl.

88. (canceled)

89. (canceled)

90. A compound of formula A-3, A-4, A-4A, A-4A1, A-4A2, and A-4A3.

91. (canceled)
